(19) United States
(12) Patent Application Publication
(10) Pub. No.: US 2022/0277805 A1
(43) Pub. Date: Sep. 1, 2022

(54) GENETIC MUTATIONAL ANALYSIS

(71) Applicants: BioSkryb Genomics, Inc., Durham, NC (US); St. Jude Children's Research Hospital, Inc., Memphis, TN (US)

(72) Inventors: Jay A.A. WEST, Durham, NC (US); Charles GAWAD, Memphis, TN (US); Sivaraman NATARAJAN, Memphis, TN (US); Robert CARTER, Memphis, TN (US); Veronica GONZALES-PENA, Memphis, TN (US)

(21) Appl. No.: 17/631,067

(22) PCT Filed: Jul. 30, 2020
(86) PCT No.: PCT/US20/44272

§ 371 Date: Jan. 28, 2022

Related U.S. Application Data
(60) Provisional application No. 62/881,180, filed on Jul. 31, 2019.

(51) Int. Cl. G16B 20/00; C12N 15/10; C12Q 1/6827; C12N 5/0781; C12N 15/11; C12N 9/22; G16B 30/10; G16H 10/40

(52) U.S. Cl. CPC G16B 20/00; C12N 15/1065; C12Q 1/6827; C12N 5/0635; C12N 15/11; C12N 9/22; G16B 30/10; G16H 10/40; C12Q 2600/156; C12Q 1/6869

(57) ABSTRACT

Provided herein are compositions and methods for accurate and scalable Primary Template-Directed Amplification (PTA) nucleic acid amplification and sequencing methods, and their applications for mutational analysis in research, diagnostics, and treatment. Such methods and compositions facilitate highly accurate amplification of target (or “template”) nucleic acids, which increases accuracy and sensitivity of downstream applications, such as Next-Generation Sequencing.

### CROSS-REFERENCE

This application claims the benefit of U.S. provisional patent application No. 62/881,180 filed on Jul. 31, 2019, which is incorporated herein by reference in its entirety.

### BACKGROUND

Research methods that utilize nucleic amplification, e.g., Next Generation Sequencing, provide large amounts of information on complex samples, genomes, and other nucleic acid sources. In some cases, these samples have been subjected to mutagenic conditions in the environment, or through gene editing technologies. There is a need for highly accurate, scalable, and efficient nucleic acid amplification and sequencing methods for research, diagnostics, and treatment involving small samples, such as those subjected to mutagenic conditions.

### BRIEF SUMMARY

Described herein are methods of detecting mutations in samples, genomes, or other nucleic acid sources.

Described herein are methods of determining a mutations comprising: (a) exposing a population of cells to a gene editing method, wherein the gene editing method utilizes reagents configured to effect a mutation in a target sequence; (b) isolating single cells from the population; (c) providing a cell lysate from a single cell; (d) contacting the cell lysate with at least one amplification primer, at least one nucleic acid polymerase, and a mixture of nucleotides, wherein the mixture of nucleotides comprises at least one terminator nucleotide which terminates nucleic acid replication by the polymerase, (d) amplifying the target nucleic acid molecule to generate a plurality of terminated amplification products, wherein the replication proceeds by strand displacement replication; (e) ligating the molecules obtained in step (e) to adaptors, thereby generating a library of amplification products; and (f) sequencing the library of amplification products, and comparing the sequences of amplification products to at least one reference sequence to identify at least one mutation. Further described herein are methods wherein the at least one mutation is present in the target sequence. Further described herein are methods wherein the at least one mutation is not present in the target sequence. Further described herein are methods wherein the gene editing method comprising use of CRISPR, TALEN, ZFN, recombinase, meganucleases, or viral integration (intentional or unintentional). Further described herein are methods wherein the gene editing technique comprises use of CRISPR. Further described herein are methods wherein the gene editing technique comprises use of a gene therapy method. Further described herein are methods wherein gene therapy method is not configured to modify somatic or germline DNA of a cell. Further described herein are methods wherein the reference sequence is a genome. Further described herein are methods wherein the reference sequence is a specificity-determining sequence, where in the specificity-determining sequence is configured to bind to the target sequence. Further described herein are methods the at least one mutation is present in a region of a sequence differing from the specificity-determining sequence by at least 1 bases. Further described herein are methods wherein the at least one mutation is present in a region of a sequence differing from the specificity-determining sequence by at least 2 bases. Further described herein are methods wherein the at least one mutation is present in a region of a sequence differing from the specificity-determining sequence by at least 3 bases. Further described herein are methods wherein the at least one mutation is present in a region of a sequence differing from the specificity-determining sequence by at least 5 bases. Further described herein are methods wherein the at least one mutation comprises an insertion, deletion, or substitution. Further described herein are methods wherein the reference sequence is the sequence of a CRISPR RNA (crRNA). Further described herein are methods wherein the reference sequence is the sequence of a single guide RNA (sgRNA). Further described herein are methods wherein the at least one mutation is present in a region of a sequence which binds to catalytically active Cas9. Further described herein are methods wherein the single cell is a mammalian cell. Further described herein are methods wherein the single cell is a human cell. Further described herein are methods wherein the single cells originate from liver, skin, kidney, blood, or lung. Further described herein are methods wherein the single cells is a primary cell. Further described herein are methods wherein the single cells is a stem cell. Further described herein are methods wherein at least some of the amplification products comprise a barcode. Further described herein are methods wherein at least some of the amplification products comprise at least two barcodes. Further described herein are methods wherein the barcode comprises a cell barcode. Further described herein are methods wherein the barcode comprises a sample barcode. Further described herein are methods wherein at least some of the amplification primers comprise a unique molecular identifier (UMI). Further described herein are methods wherein at least some of the amplification primers comprise at least two unique molecular identifiers (UMIs). Further described herein are methods wherein the method further comprises an additional amplification step using PCR. Further described herein are methods wherein the method further comprises removing at least one terminator nucleotide from the terminated amplification products prior to ligation to adapters. Further described herein are methods wherein single cells are isolated from the population using a method comprises a microfluidic device. Further described herein are methods wherein the at least one mutation occurs in less than 50% of the population of cells. Further described herein are methods wherein the at least one mutation occurs in less than 25% of the population of cells. Further described herein are methods wherein the at least one mutation occurs in less than 1% of the population of cells. Further described herein are methods wherein the at least one mutation occurs in no more than 0.1% of the population of cells. Further described herein are methods wherein the at least one mutation occurs in no more than 0.01% of the population of cells. Further described herein are methods wherein the at least one mutation occurs in no more than 0.001% of the population of cells. Further described herein are methods wherein the at least one mutation occurs in no more than 0.0001% of the population of cells. Further described herein are methods wherein the at least one mutation occurs in no more than 25% of the amplification product sequences. Further described herein are methods wherein the at least one mutation occurs in no more than 1% of the amplification product sequences. Further described herein are methods, wherein the at least one mutation occurs in no more than 0.1% of the amplification product sequences. Further described herein are methods wherein the at least one mutation occurs in no more than 0.01% of the amplification product sequences. Further described herein are methods wherein the at least one mutation occurs in no more than 0.001% of the amplification product sequences. Further described herein are methods wherein the at least one mutation occurs in no more than 0.0001% of the amplification product sequences. Further described herein are methods wherein the at least one mutation is present in a region of a sequence correlated with a genetic disease or condition. Further described herein are methods wherein the at least one mutation is present in a region of a sequence not correlated with binding of a DNA repair enzyme. Further described herein are methods wherein the at least one mutation is present in a region of a sequence not correlated with binding of MRE11. Further described herein are methods wherein the method further comprises identifying a false positive mutation previously sequenced by an alternative off-target detection method. Further described herein are methods wherein the off-target detection method is in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq.

Described herein are methods of identifying specificity-determining sequences comprising: (a) providing a library of nucleic acids, wherein at least some of the nucleic acids comprise a specificity-determining sequence; (b) performing a gene editing method on at least one cell, wherein the gene editing method comprises contacting the cell with reagents comprising at least one specificity-determining sequence; (c) sequencing a genome of the at least one cell using the method described herein, wherein the specificity-determining sequence contacted with the at least one cell is identified; and (d) identifying at least one specificity-determining sequence which provides the fewest off-target mutations. Further described herein are methods wherein the off-target mutations are synonymous or non-synonymous mutations. Further described herein are methods wherein the off-target mutations are present outside of gene coding regions.

Described herein are methods of in-vivo mutational analysis comprising: (a) performing a gene editing method on at least one cell in a living organism, wherein the gene editing method comprises contacting the cell with reagents comprising at least one specificity-determining sequence; (b) isolating at least one cell from the organism; (d) sequencing a genome of the at least one cell using a method described herein. Further described herein are methods wherein the method comprises at least two cells. Further described herein are methods further comprising identifying mutations by comparing the genome of a first cell with the genome of a second cell. Further described herein are methods wherein the first cell and the second cell are from different tissues.

Described herein are methods of predicting the age of a subject comprising: (a) providing at least one sample from the subject, wherein the at least one sample comprises a genome; (b) sequencing a genome using a method described herein to identify mutations; (c) comparing mutations obtained in step b with a standard reference curve, wherein the standard reference curve correlates mutation count and location with a verified age; and (d) predicting the age of the subject based on the mutation comparison to the standard reference curve. Further described herein are methods wherein the standard reference curve is specific for a subject's sex. Further described herein are methods wherein the standard reference curve is specific for a subject's ethnicity. Further described herein are methods wherein the standard reference curve is specific for a subject's geographic location where the subject spent a period of the subject's life. Further described herein are methods wherein the subject is less than 50 years old. Further described herein are methods wherein the subject is less than 18 years old. Further described herein are methods wherein the subject is less than 15 years old. Further described herein are methods wherein the at least one sample is more than 10 years old. Further described herein are methods wherein the at least one sample is more than 100 years old. Further described herein are methods wherein the at least one sample is more than 1000 years old. Further described herein are methods wherein at least 2 samples are sequenced. Further described herein are methods wherein at least 5 samples are sequenced. Further described herein are methods wherein the at least two samples are from different tissues.

Described herein are methods for sequencing a microbial or viral genome comprising: (a) obtaining a sample comprising one or more genomes or genome fragments; (b) sequencing the sample using the method described herein to obtain a plurality of sequencing reads; and (c) assembling and sorting the sequencing reads to generate the microbial or viral genome from even single bacterial cell or single viral particles. Further described herein are methods wherein the sample comprises genomes from at least two organisms. Further described herein are methods wherein the sample comprises genomes from at least ten organisms. Further described herein are methods wherein the sample comprises genomes from at least 100 organisms. Further described herein are methods wherein the sample origin is an environment comprising deep sea vents, ocean, mines, streams, lakes, meteorites, glaciers, or volcanoes. Further described herein are methods further comprising identifying at least one gene in the microbial genome. Further described herein are methods wherein the microbial genome corresponds to an unculturable organism. Further described herein are methods wherein the microbial genome corresponds to an symbiotic organism. Further described herein are methods further comprising cloning of the at least one gene in a recombinant host organism. Further described herein are methods wherein the recombinant host organism is a bacteria. Further described herein are methods wherein the recombinant host organism is Escherichia, Bacillus, or Streptomyces. Further described herein are methods wherein the recombinant host organism is a eukaryotic cell. Further described herein are methods wherein the recombinant host organism is a yeast cell. Further described herein are methods wherein the recombinant host organism is Saccharomyces or Pichia.

Described herein are kits for nucleic acid sequencing comprising: at least one amplification primer; at least one nucleic acid polymerase; a mixture of at least two nucleotides, wherein the mixture of nucleotides comprises at least one terminator nucleotide which terminates nucleic acid replication by the polymerase; and instructions for use of the kit to perform nucleic acid sequencing. Further described herein are kits wherein the at least one amplification primer is a random primer. Further described herein are kits wherein the nucleic acid polymerase is a DNA polymerase. Further described herein are kits wherein the DNA polymerase is a strand displacing DNA polymerase. Further described herein are kits wherein the nucleic acid polymerase is bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo−) DNA polymerase, Deep Vent DNA polymerase, Deep Vent (exo−) DNA polymerase, IsoPol DNA polymerase, DNA polymerase I, Therminator DNA polymerase, T5 DNA polymerase, Sequenase, T7 DNA polymerase, T7-Sequenase, or T4 DNA polymerase. Further described herein are kits, wherein the nucleic acid polymerase comprises 3′->5′ exonuclease activity and the at least one terminator nucleotide inhibits the 3′->5′ exonuclease activity. Further described herein are kits wherein the nucleic acid polymerase does not comprise 3′->5′ exonuclease activity. Further described herein are kits wherein the polymerase is Bst DNA polymerase, exo(−) Bst polymerase, exo(−) Bca DNA polymerase, Bsu DNA polymerase, Vent_{R} (exo−) DNA polymerase, Deep Vent (exo−) DNA polymerase, Klenow Fragment (exo−) DNA polymerase, or Therminator DNA polymerase. Further described herein are kits wherein the least one terminator nucleotide comprises modifications of the r group of the 3′ carbon of the deoxyribose. Further described herein are kits wherein the at least one terminator nucleotide is selected from the group consisting of 3′ blocked reversible terminator containing nucleotides, 3′ unblocked reversible terminator containing nucleotides, terminators containing 2′ modifications of deoxynucleotides, terminators containing modifications to the nitrogenous base of deoxynucleotides, and combinations thereof. Further described herein are kits wherein the at least one terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further described herein are kits wherein the at least one terminator nucleotide are selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further described herein are kits wherein the nucleotides with modification to the alpha group are alpha-thio dideoxynucleotides. Further described herein are kits wherein the amplification primers are 4 to 70 nucleotides in length. Further described herein are kits, wherein the at least one amplification primer is 4 to 20 nucleotides in length. Further described herein are kits wherein the at least one amplification primer comprises a randomized region. Further described herein are kits wherein the randomized region is 4 to 20 nucleotides in length. Further described herein are kits wherein the randomized region is 8 to 15 nucleotides in length. Further described herein are kits wherein the kit further comprises a library preparation kit. Further described herein are kits wherein the library preparation kit comprises one or more of: at least one polynucleotide adapter; at least one high-fidelity polymerase; at least one ligase; a reagent for nucleic acid shearing; and at least one primer. Further described herein are kits wherein the kit further comprises reagents configured for gene editing.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a workflow for detection of mutations using the PTA method, single cell sequencing, and alignment. Edited cells and non-edited control cells are amplified using PTA, sequenced using short-read sequencing, and aligned to a reference genome.

FIG. 1B illustrates the detection of small indels. Indels (black ovals) are identified by comparing the aligned sequence data to the reference genome using variant-calling software. Indels that are candidates for likely CRISPR editing events are identified by comparing indels between the edited cells and the unedited control cells, and restricting the search space to regions of the genome that show sequence similarity to the gRNA target site. Evidence for candidate editing events includes 1) indels that are located 3-4 bases upstream from the putative PAM sequences in regions of the genome that show similarity to the target site, and 2) the restriction of these indels to edited cells, with no evidence in the unedited controls cells.

FIGS. 1C and 1D illustrate the detection of translocations and large deletions. CRISPR-induced structural variants including inter- and intra-chromosomal translocations, inversions, and large deletions can be identified by comparison of read-pair mapping patterns between edited and non-edited cells. CRISPR-induced translocations are identified by read pair alignments in edited cells in which at least two regions of the read pair align to different chromosomes and the breakpoints are located in regions that show similarity to the gRNA target sequence. These discordant read pairs should not be present in the alignments of unedited cells (FIG. 1C). Large deletions are identified by read pairs that show proper orientation but contain regions that align to distant portions of the reference genome (FIG. 1D).

FIG. 1E illustrates a comparison of a prior multiple displacement amplification (MDA) method with one of the embodiments of the Primary Template-Directed Amplification (PTA) method, namely the PTA-Irreversible Terminator method.

FIG. 1F illustrates a comparison of the PTA-Irreversible Terminator method with a different embodiment, namely the PTA-Reversible Terminator method.

FIG. 1G illustrates a comparison of MDA and the PTA-Irreversible Terminator method as they relate to mutation propagation.

FIG. 1H illustrates the method steps performed after amplification, which include removing the terminator, repairing ends, and performing A-tailing prior to adapter ligation. The library of pooled cells can then undergo hybridization-mediated enrichment for all exons or other specific regions of interest prior to sequencing. The cell of origin of each read is identified by the cell barcode (shown as green and blue sequences).

FIG. 2A shows the size distribution of amplicons after undergoing PTA with addition of increasing concentrations of terminators (top gel). The bottom gel shows size distribution of amplicons after undergoing PTA with addition of increasing concentrations of reversible terminator, or addition of increasing concentrations of irreversible terminator.

FIG. 2B (GC) shows comparison of GC content of sequenced bases for MDA and PTA.

FIG. 2C shows map quality scores(e) (mapQ) mapping to human genome (p_mapped) after single cells underwent PTA or MDA.

FIG. 2D percent of reads mapping to human genome (p_mapped) after single cells underwent PTA or MDA.

FIG. 2E (PCR) shows the comparison of percent of reads that are PCR duplicates for 20 million subsampled reads after single cells underwent MDA and PTA.

FIG. 2F shows amplification kinetics as the amplicon yield vs. time (hours) for MDA, MDA no template control (NTC), PTA, and PTA no template control (NTC).

FIG. 3A shows map quality scores(c) (mapQ2) mapping to human genome (p_mapped2) after single cells underwent PTA with reversible or irreversible terminators.

FIG. 3B shows percent of reads mapping to human genome (p_mapped2) after single cells underwent PTA with reversible or irreversible terminators.

FIG. 3C shows a series of box plots describing aligned reads for the mean percent reads overlapping with Alu elements using various methods. PTA had the highest number of reads aligned to the genome.

FIG. 3D shows a series of box plots describing PCR duplications for the mean percent reads overlapping with Alu elements using the various methods.

FIG. 3E shows a series of box plots describing GC content of reads for the mean percent reads overlapping with Alu elements using various methods.

FIG. 3F shows a series of box plots describing the mapping quality of mean percent reads overlapping with Alu elements using various methods. PTA had the highest mapping quality of methods tested.

FIG. 3G shows a comparison of SC mitochondrial genome coverage breadth with different WGA methods at a fixed 7.5× sequencing depth.

FIG. 4A shows mean coverage depth of 10 kilobase windows across chromosome 1 after selecting for a high-quality MDA cell (representative of ˜50% cells) compared to a random primer PTA-amplified cell after downsampling each cell to 40 million paired reads. The figure shows that MDA has less uniformity with many more windows that have more (box A) or less (box C) than twice the mean coverage depth. There is absence of coverage in both MDA and PTA at the centromere due to high GC content and low mapping quality of repetitive regions (box B).

FIG. 4B shows plots of sequencing coverage vs. genome position for MDA and PTA methods (top). The lower box plots show allele frequencies for MDA and PTA methods as compared to the bulk sample.

FIG. 5A shows a plot of the fraction of the genome covered vs. number of reads genome to evaluate the coverage at increasing sequencing depth for a variety of methods. The PTA method approaches the two bulk samples at every depth, which is an improvement over other methods tested.

FIG. 5B shows a plot of the coefficient of variation of the genome coverage vs. number of reads to evaluate coverage uniformity. The PTA method was found to have the highest uniformity of the methods tested.

FIG. 5C shows a Lorenz plot of the cumulative fraction of the total reads vs. the cumulative fraction of the genome. The PTA method was found to have the highest uniformity of the methods tested.

FIG. 5D shows a series of box plots of calculated Gini Indices for each of the methods tested in order to estimate the difference of each amplification reaction from perfect uniformity. The PTA method was found to be reproducibly more uniform than other methods tested.

FIG. 5E shows a plot of the fraction of bulk variants called vs. number of reads. Variant call rates for each of the methods were compared to the corresponding bulk sample at increasing sequencing depth. To estimate sensitivity, the percent of variants called in corresponding bulk samples that had been subsampled to 650 million reads found in each cell at each sequencing depth (FIG. 5A) were calculated. Improved coverage and uniformity of PTA resulted in the detection of 30% more variants over the Q-MDA method, which was the next most sensitive method.

FIG. 5F shows a series of box plots of the mean percent reads overlapping with Alu elements. The PTA method significantly diminished allelic skewing at these heterozygous sites. The PTA method more evenly amplifies two alleles in the same cell relative to other methods tested.

FIG. 5G shows a plot of precision of variant calls vs. number of reads to evaluate the precision of mutation calls. Variants found using various methods which were not found in the bulk samples were considered as false positives. The PTA method resulted in the lowest false positive calls (highest precision) of methods tested.

FIG. 5H shows the fraction of false positive base changes for each type of base change across various methods. Without being bound by theory, such patterns may be polymerase dependent.

FIG. 5I shows a series of box plots of the mean percent reads overlapping with Alu elements for false positives variant calls. The PTA method resulted in the lowest allele frequencies for false positive variant calls.

FIG. 5J shows the Mean coefficient of variation (CV) of coverage at increasing bin size in a primary leukemia sample using commercial kits as an estimate of CNV calling accuracy.

FIG. 5K shows CNV profiles of PTA product from single cells for chromosomes where CNV were called in the bulk sample (shaded arrows). The unshaded arrow represents an area where subclonal CNV was suggested but not called in the bulk sample, where two of five cells were found to have the same alteration. The regions in the karyograms with decreased CNV detection represent centromeres, which showed decreased coverage in PTA-amplified cells. (for dot and line plots error bars represent one SD, for boxplots center line is the median; box limits represent upper and lower quartiles; whiskers represent 1.5× interquartile range; points show outliers).

FIG. 6A depicts a schematic description of a catalog of clonotype drug sensitivity according to the disclosure. By identifying the drug sensitivities of distinct clonotypes, a catalog can be created from which oncologists can translate clonotypes identified in a patient's tumor to a list of drugs that will best target the resistant populations.

FIG. 6B shows a change in number of leukemic clones with increasing number of leukemic cells per clone after 100 simulations. Using per cell mutation rates, simulations predict a massive diversity of smaller clones created as one cell expands into 10-100 billion cells (box A). Only the highest frequency 1-5 clones (box C) are detected with current sequencing methods. In one embodiment of the invention, methods to determine drug resistance of the hundreds of clones that are just below the level of detection of current method (box B) are provided.

FIG. 7 shows an exemplary embodiment of the disclosure. Compared to the diagnostic sample on the bottom row, culturing without chemotherapy selected for a clone (red box, lower right corner) that harbored an activating KRAS mutation. Conversely, that clone was killed by prednisolone or daunorubicin (green box, upper right corner) while lower frequency clones underwent positive selection (dashed box).

FIG. 8 is an overview of one embodiment of the disclosure, namely the experimental design for quantifying the relative sensitivities of clones with specific genotypes to specific drugs.

FIG. 9 (part A) shows beads with oligonucleotides attached with a cleavable linker, unique cell barcode, and a random primer. Part B shows a single cell and bead encapsulated in the same droplet, followed by lysis of the cell and cleavage of the primer. The droplet may then be fused with another droplet comprising the PTA amplification mix. Part C shows droplets are broken after amplification, and amplicons from all cells are pooled. The protocol according to the disclosure is then utilized for removing the terminator, end repair, and A-tailing prior to adapter ligation. The library of pooled cells then undergoes hybridization-mediated enrichment for exons of interest prior to sequencing. The cell of origin of each read is then identified using the cell barcode.

FIG. 10A demonstrates the incorporation of cellular barcodes and/or unique molecular identifiers into the PTA reactions using primers comprising cellular barcodes and/or or unique molecular identifiers.

FIG. 10B demonstrates the incorporation of cellular barcodes and/or unique molecular identifiers into the PTA reactions using hairpin primers comprising cellular barcodes and/or or unique molecular identifiers.

FIG. 11A (PTA_UMI) shows that the incorporation of unique molecular identifiers (UMIs) enables the creation of consensus reads, reducing the false positive rate caused by sequencing and other errors leading to increased sensitivity when performing germline or somatic variant calling.

FIG. 11B shows that collapsing reads with the same UMI enables the correction of amplification and other biases that could result in the false detection or limited sensitivity when calling copy number variants.

FIG. 12A shows a plot of number of mutations verses treatment groups for a direct measurement of environmental mutagenicity experiment. Single human cells were exposed to vehicle (VHC), mannose (MAN), or the direct mutagen N-ethyl-N-nitrosourea (ENU) at different treatment levels, and the number of mutations measured.

FIG. 12B shows a series of plots of the number of mutations verses different treatment groups and levels, further divided by the type of base mutations.

FIG. 12C shows a pattern representation of mutations in a trinucleotide context. Bases on the y axis are at the n−1 position, and bases on the x axis are at the n+1 position. Darker regions indicate a lower mutational frequency, and lighter regions indicate a higher mutational frequency. The solid black boxes in the top row (cytosine mutations) indicate that cytosine mutagenesis is less frequent when the cytosine is followed by a guanine. The dashed black boxes on the bottom row (thymine mutations) indicate most thymine mutations occur in positions where adenine is immediately preceding thymine.

FIG. 12D shows a graph comparing locations of known DNase I hypersensitive sites in CD34+ cells to corresponding locations from N-ethyl-N-nitrosourea treated cells. No significant enrichment of cytosine variants was observed.

FIG. 12E shows the proportion of ENU induced mutations in DNase I Hypersensitive (DH) sites. DH sites in CD34+ cells previously catalogued by the Roadmap Epigenomics Project were used to investigate whether ENU mutations are more prevalent in DH sites which represent sites of open chromatin. No significant enrichment in variant locations at DH sites was identified, and no enrichment of variants restricted to cytosines was observed in DH sites.

FIG. 12F shows a series of box plots of the proportion of ENU induced mutations in genomic locations with specific annotations. No specific enrichment was seen in specific annotations for variants (left boxes) in each cell relative to the proportion of the genome (right boxes) each annotation comprises.

FIG. 13A shows indel counts in edited vs. unedited cells within Hamming distance 7 of target site after a genome editing experiment and PTA.

FIG. 13B shows structural variant counts in edited vs. unedited cells within Hamming distance 6 of target site after a genome editing experiment and PTA.

FIG. 14A shows the detection of CRISPR-induced editing in 2 edited single cells using PTA.

FIG. 14B shows the detection of a large (>1 KB) deletion resulting from CRISPR-induced editing that is restricted to edited cell #1 using PTA.

FIG. 14C shows the detection of an inter-chromosomal translocation between chromosome 2, position 241,275,213 and chromosome 4, position 38,536,006 in edited cell #1 using PTA.

FIG. 15A shows alignment and SNV calling metrics in primary leukemia cells at increasing sequencing depth for coverage breadth (n=5 for each method, error bars represent one SD).

FIG. 15B shows alignment and SNV calling metrics in primary leukemia cells at increasing sequencing depth for CV coverage (n=5 for each method, error bars represent one SD).

FIG. 15C shows alignment and SNV calling metrics in primary leukemia cells at increasing sequencing depth for calling sensitivity (n=5 for each method, error bars represent one SD).

FIG. 15D shows alignment and SNV calling metrics in primary leukemia cells at increasing sequencing depth for SNV calling precision (n=5 for each method, error bars represent one SD).

FIG. 16A shows an overview of a kindred cell experiment where single cells are plated and cultured prior to reisolation, PTA, and sequencing of individual cells.

FIG. 16B shows methods for classifying variant types by comparing bulk and single cell data.

FIG. 16C shows SNV calling sensitivity and precision for each cell using the bulk as the standard.

FIG. 16D shows percent of variants that were called heterozygous for different variant classes.

FIG. 16E shows measured false positive and somatic variant rates in a single CD34+ human cord blood cell.

FIG. 17A shows an overview of mutation number in each sample for all variants.

FIG. 17B shows an overview of mutation number in each sample for somatic variants.

FIG. 17C shows an overview of mutation number in each sample for false positive variants.

FIG. 18A shows an overview of allele frequency distributions for germline variants.

FIG. 18B shows an overview of allele frequency distributions for somatic variants.

FIG. 18C shows an overview of allele frequency distributions for false positive variants.

FIG. 19 shows the density of homozygous or heterozygous false positive variant calls across chromosome 14 (which had the largest number of false positive calls). Mean GC content at 100 Kb intervals is running below the karyogram.

FIG. 20A shows experimental and computational methods for measuring off-target activity of genome editing strategies at single-cell resolution, where single edited cells are sequenced and indel calling is limited to sites with to five mismatches with the protospacer.

FIG. 20B shows the number of indel calls per cell. Each control or experimental cell type underwent indel calling where the target region had up to five base mismatches with either the VEGFA or EMX1 protospacer sequences. The gRNA or control listed in the key specify which gRNA that cell received. Instances where the indel is called in a genomic region that does not match the gRNA received by that cell are presumed to be false positives.

FIG. 20C shows a table of total number of off-target indel locations called that were either unique to one cell or found in multiple cells.

FIG. 20D shows genomic locations of recurrent indels with EMX1 or VEGFA gRNAs. On-target sites are noted in gray.

FIG. 20E shows circos plots of SV identified in each cell type that received either the EMX1 or VEGFA gRNA with sites that contained at least one recurrent breakpoint seen across cell types in green or only in that cell type in red. The number of SV detected per cell are plotted to the right. (for boxplots center line is the median; box limits represent upper and lower quartiles; whiskers represent 1.5× interquartile range; points show outliers).

FIG. 21 shows an experiment where removing non-recurrent single base pair insertions improved the precision of off-target detection. Each control or experimental cell type underwent indel calling requiring no more than five mismatches to either the VEGFA or EMX1 guide RNA sequence. Off-target events specifies which genomic region the gRNA had to match while the gRNA or control listed in the key specify which gRNA that cell received. Instances where the indel is called in a genomic region that does not match the gRNA received by that cell are presumed to be false positives.

FIG. 22A shows longest contig lengths for bacterial samples analyzed using the PTA method.

FIG. 22B shows graphs of each sample containing the proportion of cumulative length vs. cumulative contig length, and closest hit genus for each sample based on sequence alignment to genomes.

FIG. 22C shows a graph of bacterial sample 10 for the proportion of cumulative length vs. cumulative contig length, and closest hit genus for each sample based on sequence alignment to genomes of Haemophilus and Streptococcus.

FIG. 22D shows read pairs aligning to human chromosomes for each bacteria sample tested.

FIG. 22E shows a scheme for assigning a read as human-derived.

FIG. 22F shows read pair mapping locations for all pairs with at least one human-mapped read, for all bacteria samples tested.

FIG. 22G shows taxonomic ranks for assignment of contigs belonging to bacteria sample 10.

### DETAILED DESCRIPTION OF THE INVENTION

There is a need to develop new scalable, accurate and efficient methods for nucleic acid amplification (including single-cell and multi-cell genome amplification) and sequencing which would overcome limitations in the current methods by increasing sequence representation, uniformity and accuracy in a reproducible manner. Provided herein are compositions and methods for providing accurate and scalable Primary Template-Directed Amplification (PTA) and sequencing. Such methods and compositions facilitate highly accurate amplification of target (or “template”) nucleic acids, which increases accuracy and sensitivity of downstream applications, such as Next-Generation Sequencing. Further provided herein are methods of single nucleotide variant determination, copy number variation, structural variation, clonotyping, and measurement of environmental mutagenicity. Measurement of genome variation by PTA may be used for various applications, such as, environmental mutagenicity, predicting safety of gene editing techniques, measuring cancer treatment-mediated genomic changes, measuring carcinogenicity of compounds or radiation including genotoxicity studies for determining the safety of new foods or drugs, estimating ages, analysis of resistant bacteria, and identification of bacteria in the environment for industrial applications. Further, these methods may be used to detect selection of specific cellular populations after changes in environmental conditions, such as exposure to anti-cancer treatment, as well as to predict response to immunotherapy based on the mutation and neoantigen burden in single cancer cells.

Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574.

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, Kivioja, T. et al. Nat. Methods (2012) 9: 72-74, Brenner et al. (2000) PNAS 97(4), 1665, and Hollas and Schuler, (2003) Conference: 3rd International Workshop on Algorithms in Bioinformatics, Volume: 2812.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes.

The terms “precision” and “specificity” are in some instance used synonymously. In some instances, precision (or positive predictive value) defines the number of true positive hits divided by the total number of positive hits identified (true positives+false positives).

The term “cycle” when used in reference to a polymerase-mediated amplification reaction is used herein to describe steps of dissociation of at least a portion of a double stranded nucleic acid (e.g., a template from an amplicon, or a double stranded template, denaturation). hybridization of at least a portion of a primer to a template (annealing), and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

Methods and Applications

Described herein are methods of identifying mutations in cells with the methods of PTA. Use of the PTA method in some instances results in improvements over known methods, for example, MDA. PTA in some instances has lower false positive and false negative variant calling rates than the MDA method. Genomes, such as NA12878 platinum genomes, are in some instances used to determine if the greater genome coverage and uniformity of PTA would result in lower false negative variant calling rate. Without being bound by theory, it may be determined that the lack of error propagation in PTA decreases the false positive variant call rate. The amplification balance between alleles with the two methods is in some cases estimated by comparing the allele frequencies of the heterozygous mutation calls at known positive loci. In some instances, amplicon libraries generated using PTA are further amplified by PCR. In some instances, the PTA method identifies mutations present in single cells of a population, wherein a mutation detected by PTA occurs in less than 2%, 1%, 0.5%, 0.2%, 0.1%, 0.05%, 0.02%, 0.01%, 0.001%, 0.0001%, or less than 0.00001% of the cells in the population. In some instances, the PTA method identifies mutations in less than 2%, 1%, 0.5%, 0.2%, 0.1%, 0.05%, 0.02%, 0.01%, 0.001%, 0.0001%, or less than 0.00001% of the sequencing reads for a given base or region.

Gene Editing Safety

The continued development of genome editing tools shows great promise for improving human health; from correcting genes that result in or contribute to the formation of disease (such as sickle cell anemia, and many other diseases) to the eradication of infectious diseases that are currently incurable. However, the safety of these interventions remain unclear as a result of our incomplete understanding of how these tools interact with and permanently alter other locations in the genomes of edited cells. Methods have been developed to estimate the off-target rates of genome editing strategies, but tools that have been developed to date interrogate groups of cells together, resulting in the inability to measure the per cell off-target rates and variance in off-target activity between cells, as well as to detect rare editing events that occur in a small number of cells. These suboptimal strategies for measuring genome editing fidelity have resulted in a limited capacity to determine the sensitivity and precision of a given genome editing approach.

Gene therapy methods may comprise modification of a mutated, disease causing gene, knockout of a disease causing gene, or introduction of a new gene in cells. Such approaches in some instances comprise modification of genomic DNA. In other instances, viral or other delivery systems are configured such that they do not integrate or modify genomic DNA in cells. However, such systems may nevertheless produce unwanted or unexpected modifications to somatic or germline DNA. Taking advantage of the improved variant calling sensitivity and precision of PTA in single cells, quantitative measurements of unintended insertion rates of gene therapy approaches with high sensitivity in single cells in some instances is conducted. The method is some cases detects the insertion of specific sequences in a non-desired location by detecting the surrounding sequence to determine if the gene therapy approach is causes insertion or modification of the host genome.

Described herein are methods of identifying mutations and structural modifications (i.e. translocation, insertions and deletions) in animal, plant or microbial cells that have undergone genome editing (e.g., CRISPR (Clustered regularly interspaced short palindromic repeats), TALEN (Transcription activator-like effector nucleases), ZFN (Zinc finger nucleases), recombinase, meganucleases, viral integration, or other genome editing technologies). In some embodiments, genome editing is unintentional, or is a secondary effect of another process. In some instances, genome editing comprises site-specific or targeted genome editing. Such cells in some instances can be isolated and subjected to PTA and sequencing to determine mutation burden, mutation combination and structural variation in each cell. The per-cell mutation rate and locations of mutations that result from a genome editing protocol are in some instances used to assess the safety and/or efficiency of a given genome editing method. Identification of mutations in some instances comprises comparing sequencing data obtained using the PTA method with a reference sequence. In some instances, the reference sequence is a genome. In some instances, at least one mutation is identified by PTA after a gene editing process. In some instances, the reference sequence is a specificity-determining sequence which promotes introduction of a mutation into a target sequence of a nucleic acid. In some instances, at least one mutation is identified by PTA after a gene editing process, wherein the mutation is located in the target sequence. In some instances, off-target mutation rates are analyzed by identifying at least one mutation not in the target sequence. Although some areas of a nucleic acid may be predicted to suffer off-target mutation based on sequence homology to target sequences, regions with lower homology may also have off-target mutations. In some instances, the PTA method identifies a mutation in an off-target region of a sequence comprising at least 3, 4, 5, 6, 7, or 8 base mismatches with the target sequence or reverse complement thereof. In some instances, single cells are analyzed with PTA. In some instances, populations of cells are analyzed with PTA.

Many current methods of mutational analysis obtain sequencing data on bulk cell populations. However, such approaches provide limited information regarding the actual frequency of mutations in the population, Single cell analysis using PTA in some instances provides much higher resolution of the off target rate of insertion, strand breaks (resulting in mutation), and translocation as the number of cells (i.e. a single cell) is known. PTA, which has a known rate of variation detection, in a known number of single cells, allows the method in some instances to accurately determine the per cell frequency and combinations of alterations in a population of cells. In some instances, at least 10, 100, 1000, 10,000, 100,000, or more than 100,000 single cells are analyzed with PTA to establish a rate of variation. In some instances, no more than 10, 100, 1000, 10,000, 100,000, or no more than 100,000 single cells are analyzed with PTA to establish a rate of variation. In some instances, 10-1000, 50-5000, 100-100,000, 1000-100,000, 100-1,000,000, or 100-10,000 single cells are analyzed with PTA to establish a rate of variation. In some instances, mutations identified by analysis of one or more single cells are not identified or detected from bulk sequencing of the population of cells.

CRISPR may be used to introduce mutations into one or more cells, such as mammalian cells which are then analyzed by PTA. In some instances, the specificity-determining sequence is present in a CRISPR RNA (crRNA) or single guide RNA (sgRNA). In some instances, the mammalian cells are human cells. In some instances, the cells originate from liver, skin, kidney, blood, or lung. In some instances, the cells are primary cells. In some instances, the cells are stem cells. Previously reported methods of identifying off-target mutations generated from CRISPR have included pulldown of sequences binding to catalytically active Cas9, however this may lead to false positives as mutations are not introduced at all Cas9 binding sites. In some instances, the PTA method identifies at least one mutation present in a region of a sequence which binds to catalytically active Cas9. In some instances, the PTA method results in fewer false positives for at least one mutation present in a region of a sequence which binds to catalytically active Cas9.

Described herein are methods of identifying mutations in animal, plant or microbial cells that have undergone genome editing (e.g., CRISPR, TALEN, ZFN, recombinase, meganucleases, viral integration, or other technologies), wherein the method comprises amplification of a genomic or fragment thereof in the presence of at least one terminator nucleotide. In some instances, amplification with the terminator takes place in solution. In some instances, one of either at least one primer or at least one genomic fragment is attached to a surface. In some instances, at least one primer is attached to a first solid support, and at least one genomic fragment is attached to a second solid support, wherein the first solid support and the second solid support are not connected. In some instances, at least one primer is attached to a first solid support, and at least one genomic fragment is attached to a second solid support, wherein the first solid support and the second solid support are not the same solid support. In some instances, the method comprises amplification of a genomic or fragment thereof in the presence of at least one terminator nucleotide, wherein the number of amplification cycles is less than 12, 10, 9, 8, 7, 6, 5, 4, or less than 3 cycles. In some instances, the average length of amplification products is 100-1000, 200-500, 200-700, 300-700, 400-1000, or 500-1200 bases in length. In some instances, the method comprises amplification of a genomic or fragment thereof in the presence of at least one terminator nucleotide, wherein the number of amplification cycles is no more than 6 cycles. In some instances, the at least one terminator nucleotide does comprise a detectable label or tag. In some instances, the amplification comprises 2, 3, or 4 terminator nucleotides. In some instances, at least two of the terminator nucleotides comprise a different base. In some instances, at least three of the terminator nucleotides comprise a different base. In some instances, four terminator nucleotides each comprise a different base.

Described herein are methods for determining the safety of gene therapies. In some instances, the functions of a cell are modified through a gene editing or other expression method. In some instances, viral delivery systems to change cellular functions are configured such that they do not integrate into the genome of the cell. In some instances the PTA method is used to identify unexpected or unwanted changes to cell genomes. In some instances, PTA is used to identify mutations to somatic or germline DNA that result from gene therapy.

Clonal Analysis of Tumor Cells

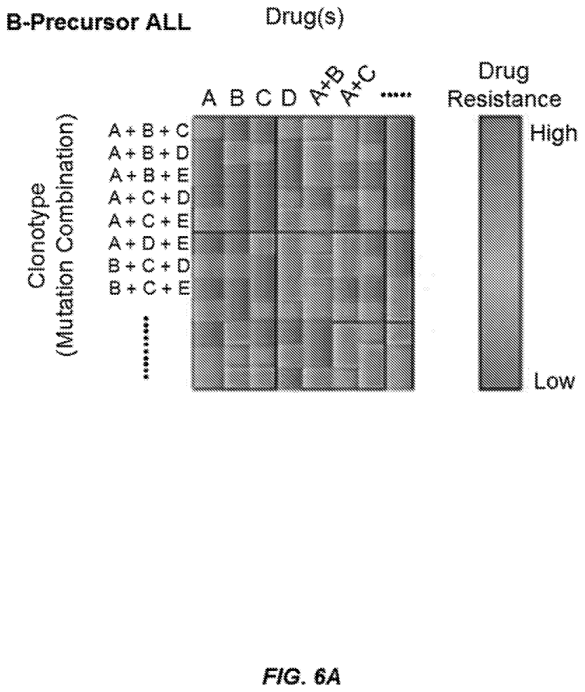

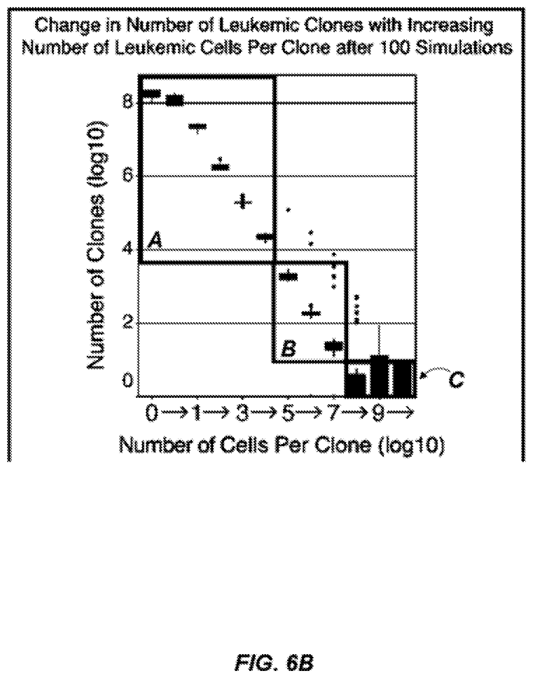

Cells analyzed using the methods described herein in some instances comprise tumor cells. For example, circulating tumor cells can be isolated from a fluid taken from patients, such as but not limited to, blood, bone marrow, urine, saliva, cerebrospinal fluid, pleural fluid, pericardial fluid, ascites, or aqueous humor. The cells are then subjected to the methods described herein (e.g. PTA) and sequencing to determine mutation burden and mutation combination in each cell. These data are in some instances used for the diagnosis of a specific disease or as tools to predict treatment response. Similarly, in some instances cells of unknown malignant potential in some instances are isolated from fluid taken from patients, such as but not limited to, blood, bone marrow, urine, saliva, cerebrospinal fluid, pleural fluid, pericardial fluid, ascites, aqueous humor, blastocoel fluid, or collection media surrounding cells in culture. In some instances, a sample is obtained from collection media surrounding embryonic cells. After utilizing the methods described herein and sequencing, such methods are further used to determine mutation burden and mutation combination in each cell. These data are in some instances used for the diagnosis of a specific disease or as tools to predict progression of a premalignant state to overt malignancy. In some instances, cells can be isolated from primary tumor samples. The cells can then undergo PTA and sequencing to determine mutation burden and mutation combination in each cell. These data can be used for the diagnosis of a specific disease or are as tools to predict the probability that a patient's malignancy is resistant to available anti-cancer drugs. By exposing samples to different chemotherapy agents, it has been found that the major and minor clones have differential sensitivity to specific drugs that does not necessarily correlate with the presence of a known “driver mutation,” suggesting that combinations of mutations within a clonal population determine its sensitivities to specific chemotherapy drugs. Without being bound by theory, these findings suggest that a malignancy may be easier to eradicate if premalignant lesions that have not yet expanded are and evolved into clones are detected whose increased number of genome modification may make them more likely to be resistant to treatment. See, Ma et al., 2018, “Pan-cancer genome and transcriptome analyses of 1,699 pediatric leukemias and solid tumors.” A single-cell genomics protocol is in some instances used to detect the combinations of somatic genetic variants in a single cancer cell, or clonotype, within a mixture of normal and malignant cells that are isolated from patient samples. This technology is in some instances further utilized to identify clonotypes that undergo positive selection after exposure to drugs, both in vitro and/or in patients. As shown in FIG. 6A, by comparing the surviving clones exposed to chemotherapy compared to the clones identified at diagnosis, a catalog of cancer clonotypes can be created that documents their resistance to specific drugs. PTA methods in some instances detect the sensitivity of specific clones in a sample composed of multiple clonotypes to existing or novel drugs, as well as combinations thereof, where the method can detect the sensitivity of specific clones to the drug. This approach in some instances shows efficacy of a drug for a specific clone that may not be detected with current drug sensitivity measurements that consider the sensitivity of all cancer clones together in one measurement. When the PTA described herein are applied to patient samples collected at the time of diagnosis in order to detect the cancer clonotypes in a given patient's cancer, a catalog of drug sensitivities may then be used to look up those clones and thereby inform oncologists as to which drug or combination of drugs will not work and which drug or combination of drugs is most likely to be efficacious against that patient's cancer. The PTA may be used for analysis of samples comprising groups of cells. In some instances, a sample comprises neurons or glial cells. In some instances, the sample comprises nuclei.

Clinical and Environmental Mutagenesis

Described herein are methods of measuring the mutagenicity of an environmental factor. For example, cells (single or a population) are exposed to a potential environmental condition. For example, cells such originating from organs (liver, pancreas, lung, colon, thyroid, or other organ), tissues (skin, or other tissue), blood, or other biological source are in some instances used with the method. In some instances, an environmental condition comprises heat, light (e.g. ultraviolet), radiation, a chemical substance, or any combination thereof. After an amount of exposure to the environmental condition, in some instances minutes, hours, days, or longer, single cells are isolated and subjected to the PTA method. In some instances, molecular barcodes and unique molecular identifiers are used to tag the sample. The sample is sequenced and then analyzed to identify mutations resulting from exposure to the environmental condition. In some instances, such mutations are compared with a control environmental condition, such as a known non-mutagenic substance, vehicle/solvent, or lack of an environmental condition. Such analysis in some instances not only provides the total number of mutations caused by the environmental condition, but also the locations and nature of such mutations. Patterns are in some instances identified from the data, and may be used for diagnosis of diseases or conditions. In some instances, patterns are used to predict future disease states or conditions. In some instances, the methods described herein measure the mutation burden, locations, and patterns in a cell after exposure to an environmental agent, such as, e.g., a potential mutagen or teratogen. This approach in some instances is used to evaluate the safety of a given agent, including its potential to induce mutations that can contribute to the development of a disease. For example, the method could be used to predict the carcinogenicity or teratogenicity of an agent to specific cell types after exposure to a specific concentration of the specific agent. In some instances, the agent is a medicine or drug. In some instances, the agent is a food. In some instances, the agent is a genetically modified food. In some instances, the agent is a pesticide or other agricultural chemical. In some instances, the location and rate of mutations is used to predict the age of an organism. Such methods are in some instances performed on samples that are hundreds, thousands, or tens of thousands of years old. Mutational patterns are in some cases compared with other data methods such as carbon dating to generate standard curves. In some instances the age of a human is determined by comparison of mutational numbers and patterns from a sample.

Described herein are methods of determining mutations in cells that are used for cellular therapy, such as but not limited to the transplantation of induced pluripotent stem cells, transplantation of hematopoietic or other cells that have not be manipulated, or transplantation of hematopoietic or other cells that have undergone genome edits. The cells can then undergo PTA and sequencing to determine mutation burden and mutation combination in each cell. The per-cell mutation rate and locations of mutations in the cellular therapy product can be used to assess the safety and potential efficacy of the product, including measurement of neoantigen burden.

Microbial Samples

Described herein are methods of analyzing microbial samples. In another embodiment, microbial cells (e.g., bacteria, fungi, protozoa) can be isolated from plants or animals (e.g., from microbiota samples [e.g., GI microbiota, skin microbiota, etc.] or from bodily fluids such as, e.g., blood, bone marrow, urine, saliva, cerebrospinal fluid, pleural fluid, pericardial fluid, ascites, or aqueous humor). In addition, microbial cells may be isolated from indwelling medical devices, such as but not limited to, intravenous catheters, urethral catheters, cerebrospinal shunts, prosthetic valves, artificial joints, or endotracheal tubes. The cells can then undergo PTA and sequencing to determine the identity of a specific microbe, as well as to detect the presence of microbial genetic variants that predict response (or resistance) to specific antimicrobial agents. These data can be used for the diagnosis of a specific infectious disease and/or as tools to predict treatment response. In some instances, single microbial cells are analyzed for mutations. In one embodiment, PTA is used to identify microorganisms with high value for industrial applications, such as production of biofuels or environmental restoration (oil spill cleanup, CO2 sequestration/removal). In some instances, microbial samples are obtained from extreme environments, such as deep sea vents, ocean, mines, streams, lakes, meteorites, glaciers, or volcanoes. In some instances, microbial samples comprise strains of microbes that are “unculturable” in the laboratory under standard conditions. Sequencing of microbial samples prepared using PTA in some instances comprises obtaining sequencing reads for assembly into contigs. In some instances no more than 0.1, 0.5, 1, 1.5, 2, 3, 5, 8, or 10 million reads are obtained. Analysis and identification of microbial samples in some instances comprises comparing assembled contigs to known microbial genome reference sequences. In some instances, the largest assembled contig is used for comparison to reference sequences. In some instances, reads are filtered which map to one or more genes in human genomic DNA. In some instances, filtering occurs if both reads (forward and backward) map to a human gene. In some instances, filtering occurs if at least one read (forward or backward) map to a human gene. In some instances, the human gene is GRCh38. In some instances, an assembly-free identification method is used with PTA. In some instances, assembly-free methods such as Kraken are used. In some instances, assembly-free methods comprise assigning reads to taxa based on k-mers using a reference database.

Fetal Cells

Cells for use with the PTA method may be fetal cells, such as embryonic cells. In some embodiments, PTA is used in conjunction with non-invasive preimplantation genetic testing (NIPGT). In a further embodiment, cells can be isolated from blastomeres or blastocytes that are created by in vitro fertilization. The cells can then undergo PTA (e.g., nucleic acids in the cells are amplified with PTA) and sequencing to determine the burden and combination of potentially disease predisposing genetic variants in each cell. The mutation profile of the cell can then be used to extrapolate the genetic predisposition of the blastomere to specific diseases prior to implantation. In some instances embryos in culture shed nucleic acids that are used to assess the health of the embryo using low pass genome sequencing. In some instances, embryos are frozen-thawed. In some instances, nucleic acids obtained from blastocyte culture conditioned medium (BCCM), blastocoel fluid (BF), or a combination thereof. In some instances, PTA analysis of fetal cells is used to detect chromosomal abnormalities, such as fetal aneploidy. In some instances, PTA is used to detect diseases such as Down's or Patau syndromes. In some instances, frozen blastocytes are thawed and cultured for a period of time before obtaining nucleic acids for analysis (e.g., culture media, BF, or a cell biopsy). In some instances, blastocytes are cultured for no more than 4, 6, 8, 12, 16, 24, 36, 48, or no more than 64 hours prior to obtaining nucleic acids for analysis.

Mutations

In some instances, the methods (e.g., PTA) described herein result in higher detection sensitivity and/or lower rates of false positives for the detection of mutations. In some instances a mutation is a difference between an analyzed sequence (e.g., using the methods described herein) and a reference sequence. Reference sequences are in some instances obtained from other organisms, other individuals of the same or similar species, populations of organisms, or other areas of the same genome. In some instances, mutations are identified on a plasmid or chromosome. In some instances, a mutation is an SNV (single nucleotide variation), SNP (single nucleotide polymorphism), or CNV (copy number variation, or CNA/copy number aberration). In some instances, a mutation is base substitution, insertion, or deletion. In some instances, a mutation is a transition, transversion, nonsense mutation, silent mutation, synonymous or non-synonymous mutation, non-pathogenic mutation, missense mutation, or frameshift mutation (deletion or insertion). In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq.

Primary Template-Directed Amplification

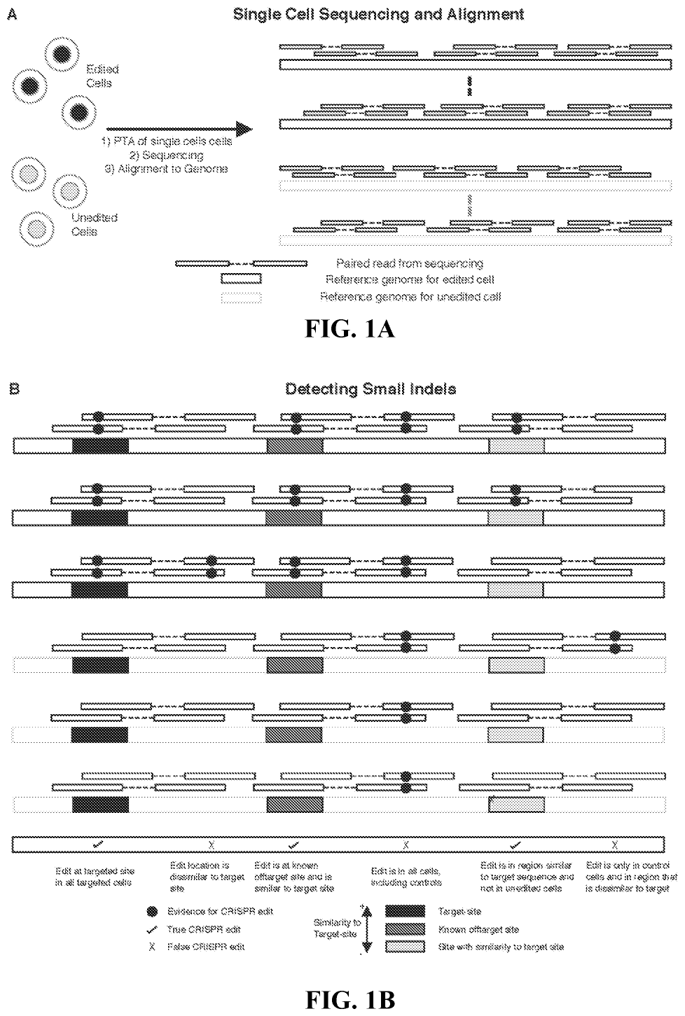

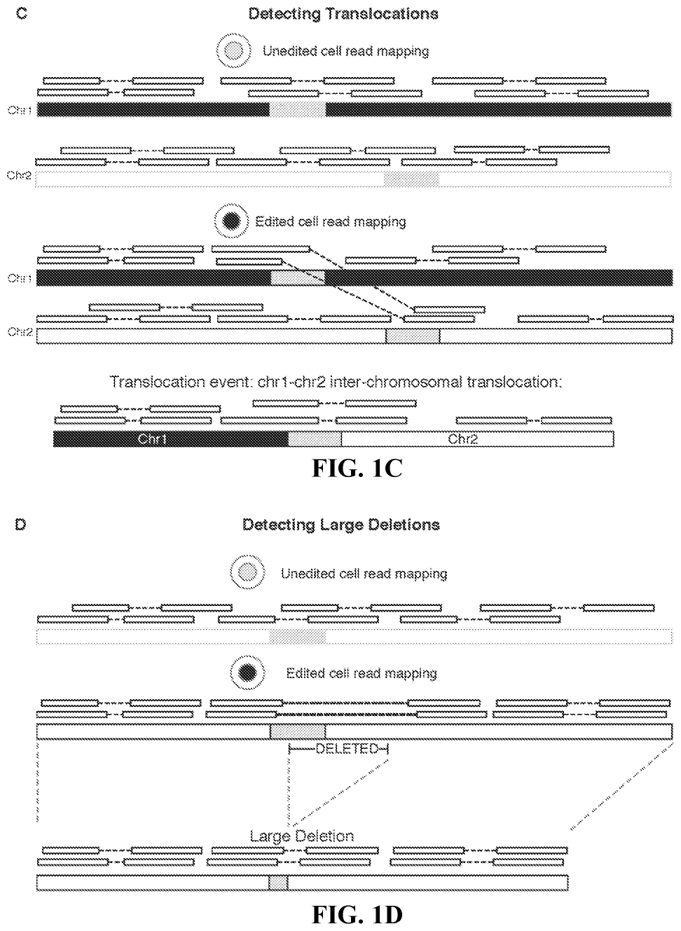

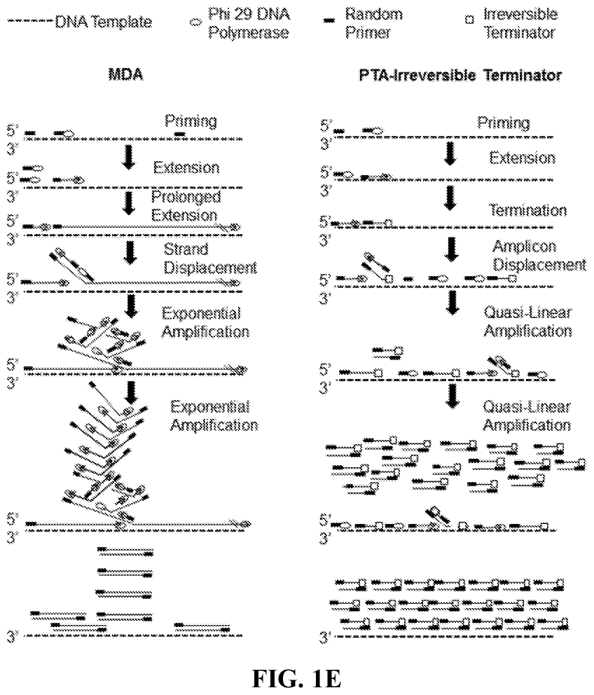

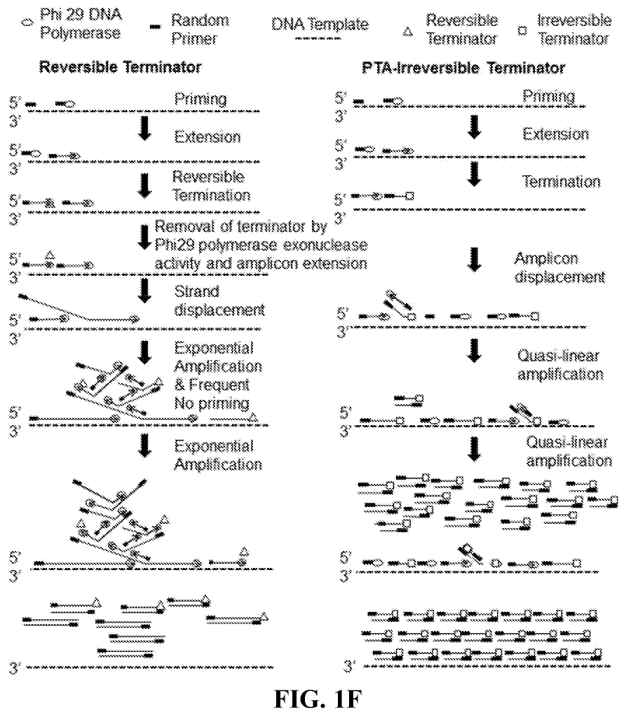

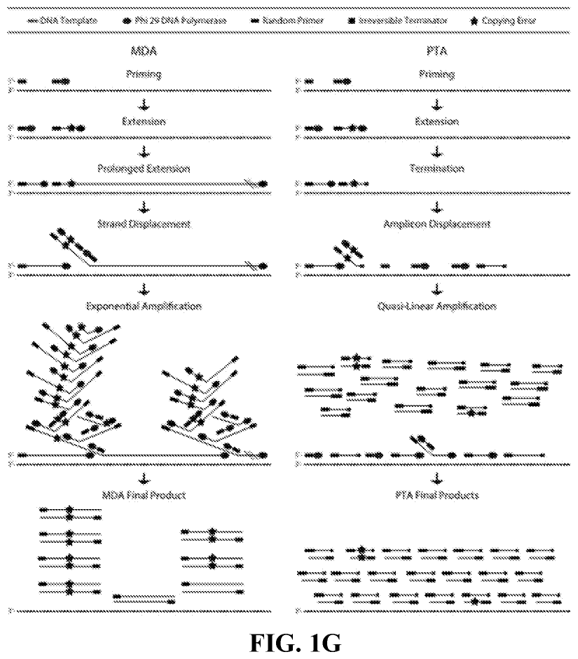

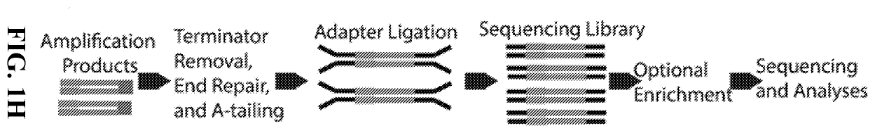

Described herein are nucleic acid amplification methods, such as “Primary Template-Directed Amplification (PTA).” For example, the PTA methods described herein are schematically represented in FIGS. 1A-1H. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. Moreover, the terminated amplification products can undergo direction ligation after removal of the terminators, allowing for the attachment of a cell barcode to the amplification primers so that products from all cells can be pooled after undergoing parallel amplification reactions (FIG. 1F). In some instances, terminator removal is not required prior to amplification and/or adapter ligation.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (Φ29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (Φ29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo(−) Bst; Aliotta et al., Genet. Anal. (Netherlands) 12:185-195 (1996)), exo(−)Bca DNA polymerase (Walker and Linn, Clinical Chemistry 42:1604-1608 (1996)), Bsu DNA polymerase, Vent_{R} DNA polymerase including Vent_{R} (exo−) DNA polymerase (Kong et al., J. Biol. Chem. 268:1965-1975 (1993)), Deep Vent DNA polymerase including Deep Vent (exo−) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(−) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67(12):7648-7653 (1993)), adenovirus DNA-binding protein (Zijderveld and van der Vliet, J. Virology 68(2): 1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, T4 UvsY, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein. In some instances, amplification is conducted using a polymerase and a nicking enzyme (e.g., “NEAR”), such as those described in U.S. Pat. No. 9,617,586. In some instances, the nicking enzyme is Nt.BspQI, Nb.BbvCi, Nb.BsmI, Nb.BsrDI, Nb.BtsI, Nt.AlwI, Nt.BbvCI, Nt.BstNBI, Nt.CviPII, Nb.Bpu10I, or Nt.Bpu10I.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10(4), 351)

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI) (see FIGS. 1H, 2B-3E, 9, 10A, and 10B).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of terminator to non-terminator nucleotides is modified for the amount of template present or the size of the template. In some instances, the ratio of ratio of terminator to non-terminator nucleotides is reduced for smaller samples sizes (e.g., femtogram to picogram range). In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100:1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′ exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo−) DNA polymerase, Klenow Fragment (exo−) DNA polymerase, Therminator DNA polymerase, and Vent_{R} (exo−).

Primers and Amplicon Libraries

Described herein are amplicon libraries resulting from amplification of at least one target nucleic acid molecule. Such libraries are in some instances generated using the methods described herein, such as those using terminators. Such methods comprise use of strand displacement polymerases or factors, terminator nucleotides (reversible or irreversible), or other features and embodiments described herein. In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of PCR amplification cycles. In some instances, no more than 30, 25, 20, 15, 13, 11, 10, 9, 8, 7, 6, 5, 4, or 3 PCR cycles are used to generate copies of the target nucleic acid molecule. In some instances, about 30, 25, 20, 15, 13, 11, 10, 9, 8, 7, 6, 5, 4, or about 3 PCR cycles are used to generate copies of the target nucleic acid molecule. In some instances, 3, 4, 5, 6, 7, or 8 PCR cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 PCR cycles are used to generate copies of the target nucleic acid molecule. Amplicon libraries generated using the methods described herein are in some instances subjected to additional steps, such as adapter ligation and further PCR amplification. In some instances, such additional steps precede a sequencing step.

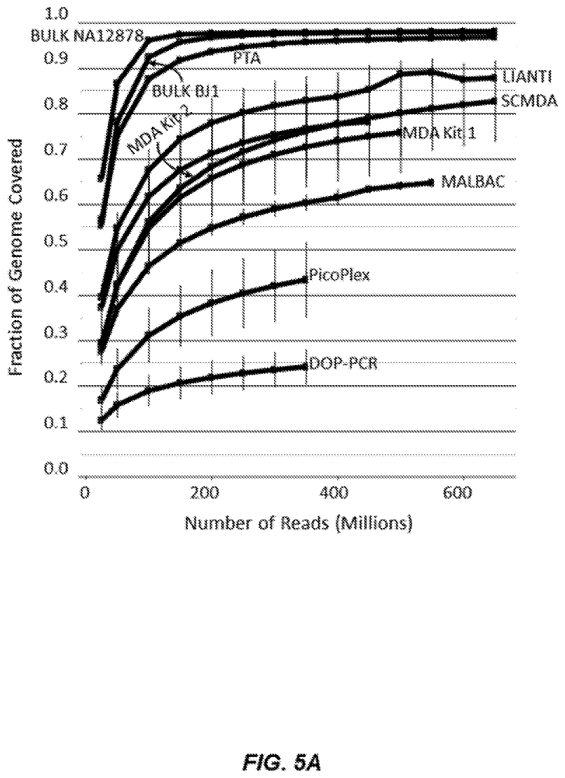

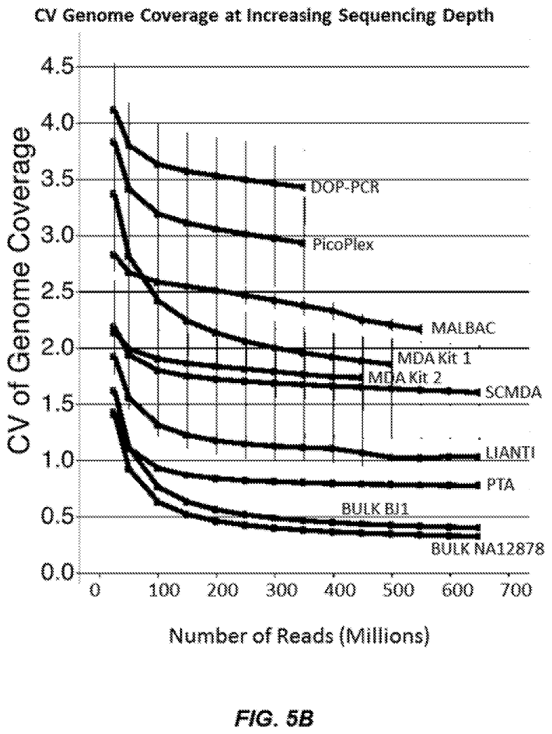

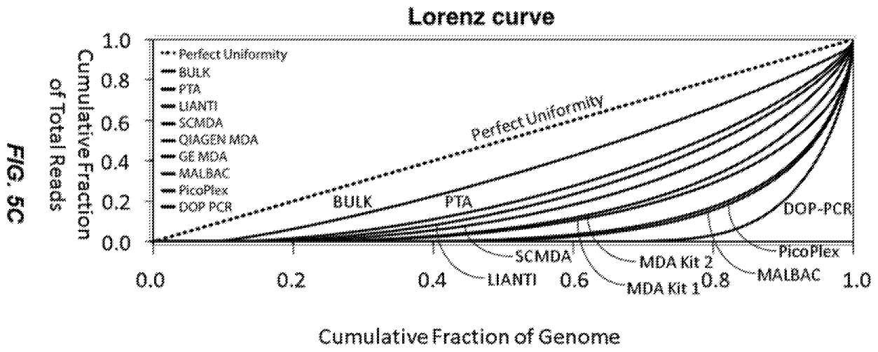

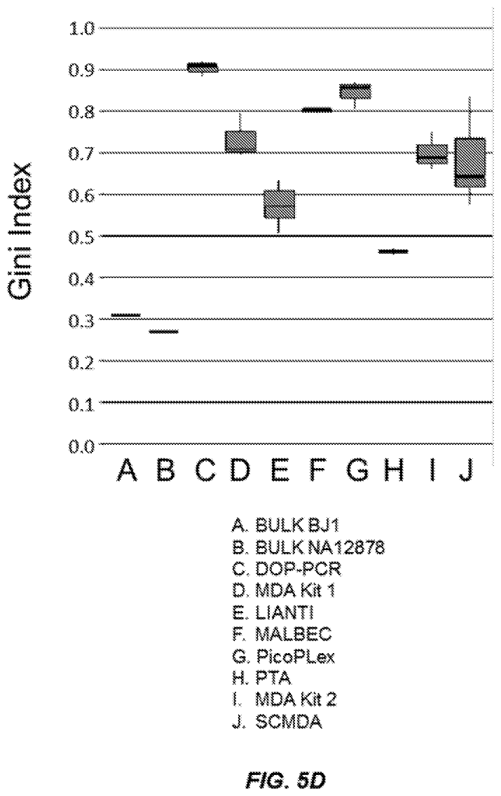

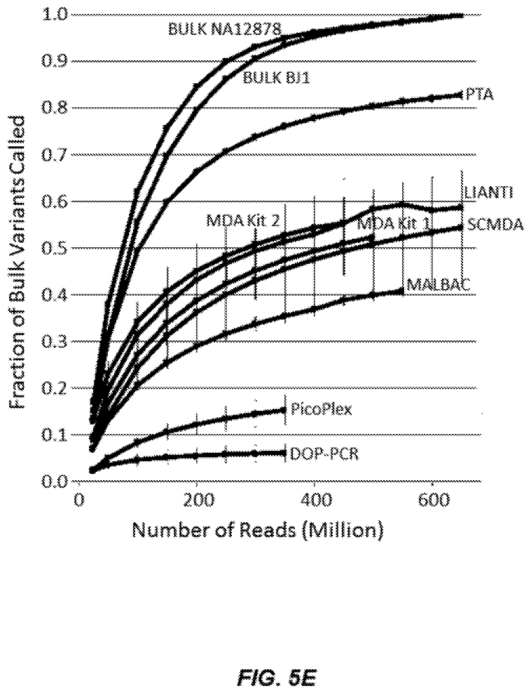

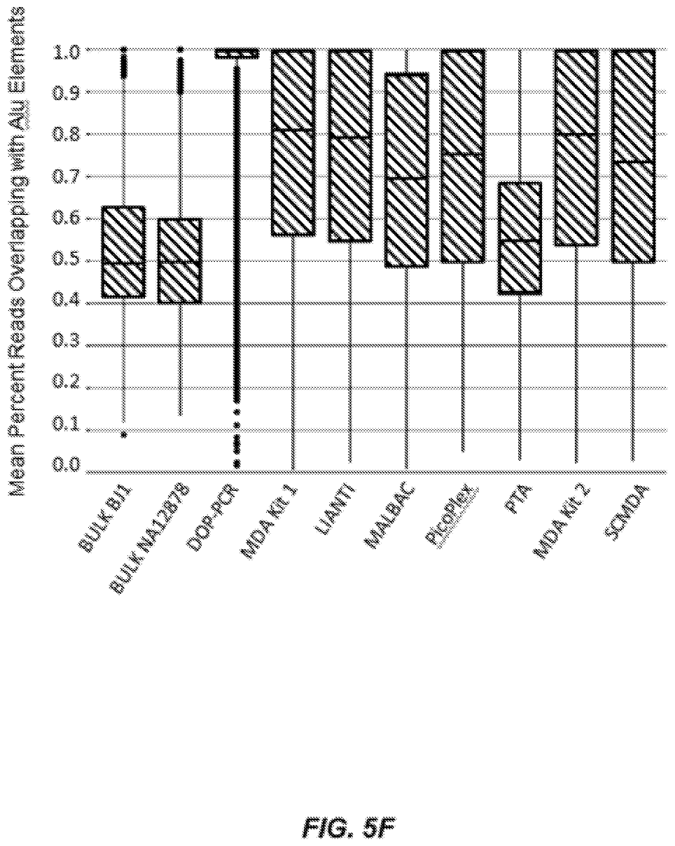

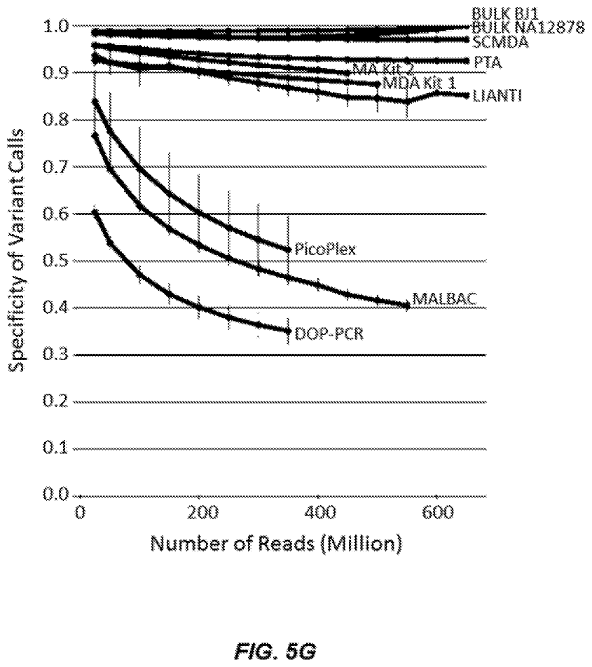

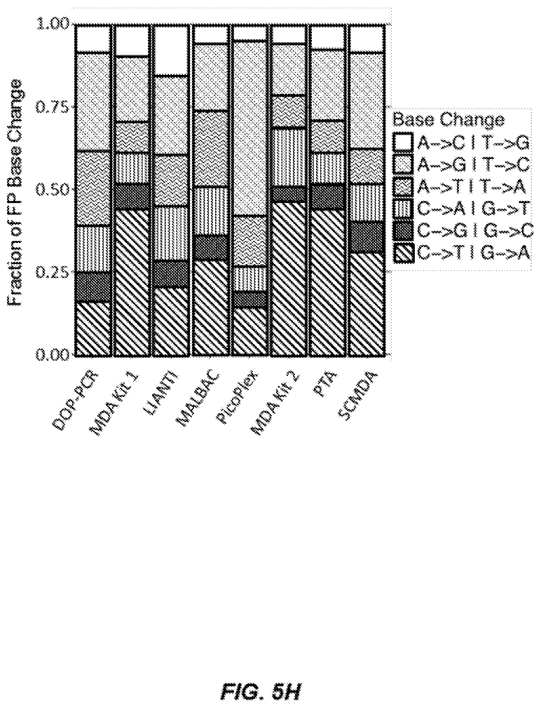

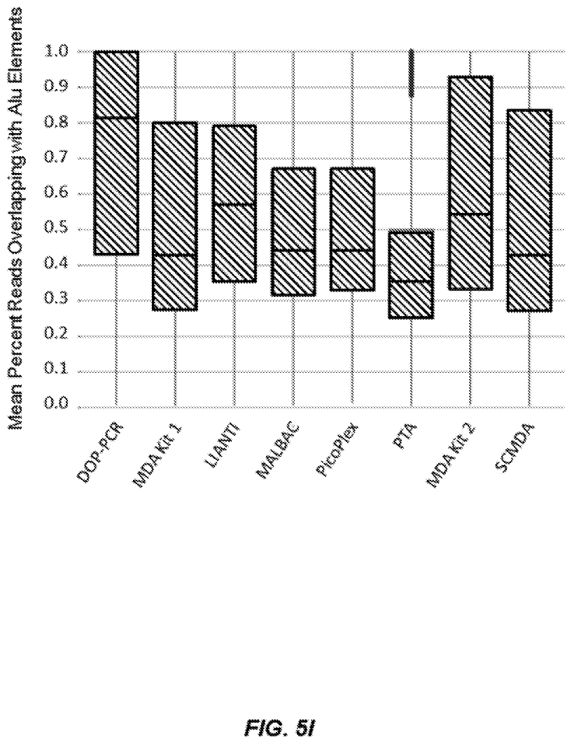

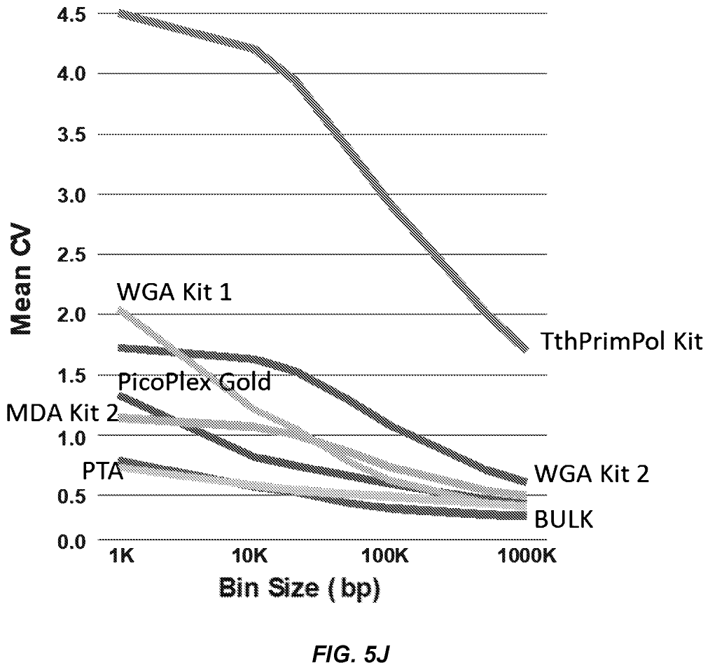

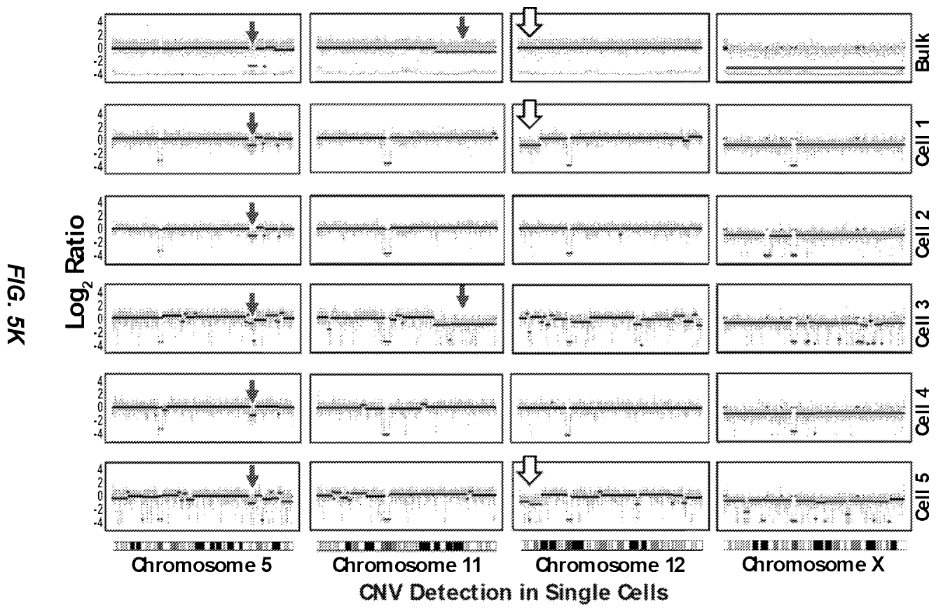

Amplicon libraries of polynucleotides generated from the PTA methods and compositions (terminators, polymerases, etc.) described herein in some instances have increased uniformity. Uniformity, in some instances, is described using a Lorenz curve (e.g., FIG. 5C), or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example no more than 100 million, 200 million, 300 million, 400 million, or no more than 500 million reads are obtained. In some instances, the read length is about 50,75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process (FIG. 1A). In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential ligation and amplification of smaller fragments during PCR while preparing sequencing libraries, as well as a result of the preferential formation of clusters from smaller sequencing library fragments during sequencing (e.g., sequencing by synthesis, nanopore sequencing, or other sequencing method). Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites. In some instances, libraries are prepared by fragmenting nucleic acids mechanically or enzymatically. In some instances, libraries are prepared using tagmentation via transposomes. In some instances, libraries are prepared via ligation of adapters, such as Y-adapters, universal adapters, or circular adapters.

The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, extracted nucleic acid from individual cells are contacted with one or more beads having a unique set of sample barcodes and/or UMI sequences in order to identify the extracted nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, extracted nucleic acid from individual cells are contacted with a droplet having a unique set of sample barcodes and/or UMI sequences in order to identify the extracted nucleic acid from the individual cell. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, Calif., USA, and Illumina, San Diego, Calif., USA)); Chromium (10× Genomics, Pleasanton, Calif., USA)); Rhapsody Single-Cell Analysis System (BD, Franklin Lakes, N.J., USA); Tapestri Platform (MissionBio, San Francisco, Calif., USA)), Nadia Innovate (Dolomite Bio, Royston, UK); C1 and Polaris (Fluidigm, South San Francisco, Calif., USA); ICELL8 Single-Cell System (Takara); MSND (Wafergen); Puncher platform (Vycap); CellRaft AIR System (CellMicrosystems); DEPArray NxT and DEPArray System (Menarini Silicon Biosystems); AVISO CellCelector (ALS); InDrop System (1CellBio), and TrapTx (Celldom).

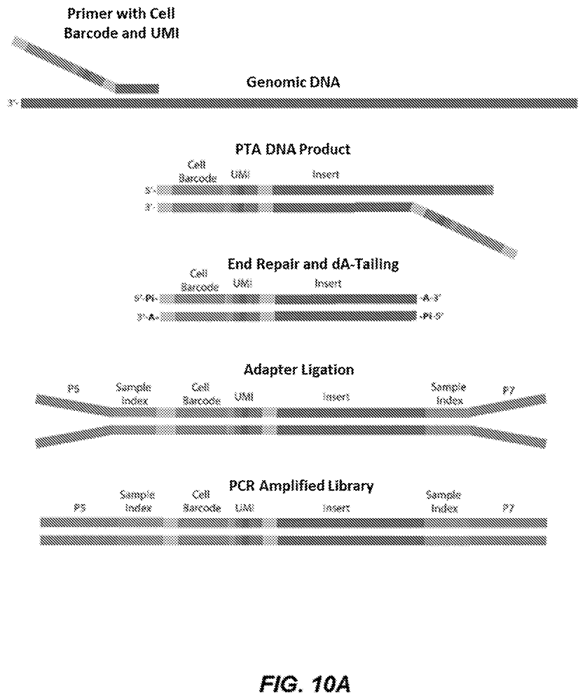

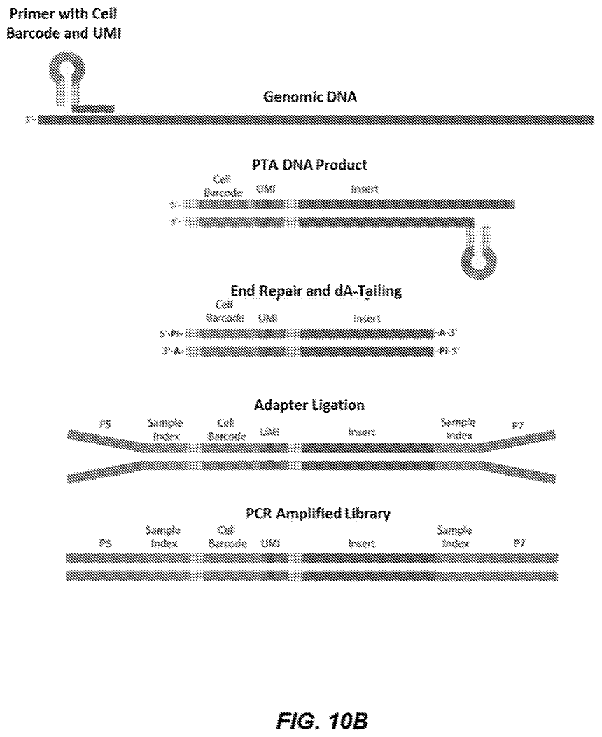

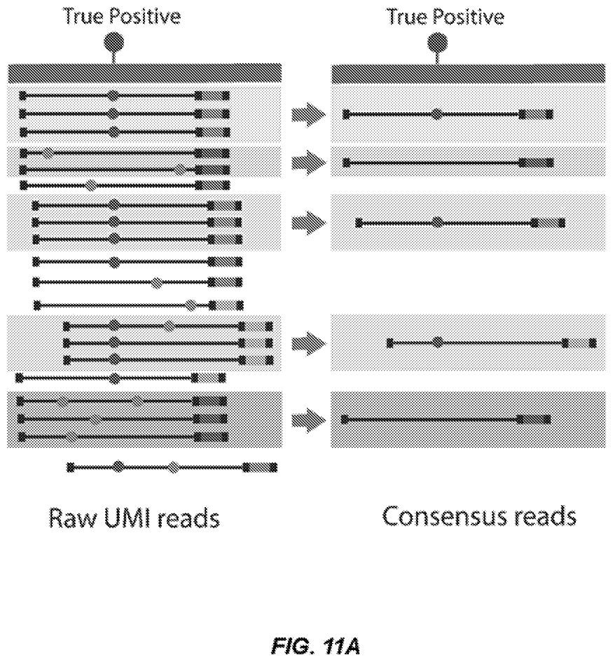

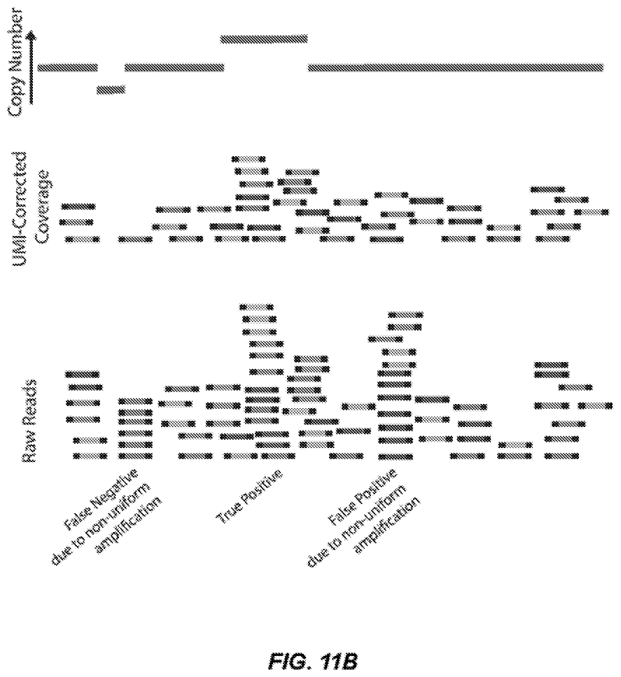

PTA primers may comprise a sequence-specific or random primer, an address tag, a cell barcode and/or a unique molecular identifier (UMI) (see, e.g., FIGS. 10A (linear primer) and 10B (hairpin primer)). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 10⁶, 10⁷, 10⁸, 10⁹, or at least 10¹⁰ unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection (FIGS. 11A and 11B). In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S Pat. No. 8,835,358 discloses the principle of digital counting after attaching a random amplifiable barcode. Schmitt. et al and Fan et al. (vide supra) disclose similar methods of correcting sequencing errors.

The methods described herein may further comprise additional steps, including steps performed on the sample or template. Such samples or templates in some instance are subjected to one or more steps prior to PTA. In some instances, samples comprising cells are subjected to a pre-treatment step. For example, cells undergo lysis and proteolysis to increase chromatin accessibility using a combination of freeze-thawing, Triton X-100, Tween 20, and Proteinase K. Other lysis strategies are also be suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCl, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified according to the methods described herein may be sequenced and analyzed using methods known to those of skill in the art. Non-limiting examples of the sequencing methods which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLiD, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem.47:164-172). In some instances, the amplified nucleic acid molecules are shotgun sequenced. Sequencing of the sequencing library is in some instances performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis (array/colony-based or nanoball based).

Described herein are methods generating amplicon libraries from samples comprising short nucleic acid using the PTA methods described herein. In some instances, PTA leads to improved fidelity and uniformity of amplification of shorter nucleic acids. In some instances, nucleic acids are no more than 2000 bases in length. In some instances, nucleic acids are no more than 1000 bases in length. In some instances, nucleic acids are no more than 500 bases in length. In some instances, nucleic acids are no more than 200, 400, 750, 1000, 2000 or 5000 bases in length. In some instances, samples comprising short nucleic acid fragments include but at not limited to ancient DNA (hundreds, thousands, millions, or even billions of years old), FFPE (Formalin-Fixed Paraffin-Embedded) samples, cell-free DNA, or other sample comprising short nucleic acids.

### Kits

Described herein are kits facilitating the practice of the PTA method. Various combinations of the components set forth above in regard to exemplary reaction mixtures and reaction methods can be provided in a kit form. A kit may include individual components that are separated from each other, for example, being carried in separate vessels or packages. A kit in some instances includes one or more sub-combinations of the components set forth herein, the one or more sub-combinations being separated from other components of the kit. The sub-combinations in some instances are combinable to create a reaction mixture set forth herein (or combined to perform a reaction set forth herein). In particular embodiments, a sub-combination of components that is present in an individual vessel or package is insufficient to perform a reaction set forth herein. However, the kit as a whole in some instances includes a collection of vessels or packages the contents of which can be combined to perform a reaction set forth herein.

A kit can include a suitable packaging material to house the contents of the kit. The packaging material in some instances is constructed by well-known methods, preferably to provide a sterile, contaminant-free environment. The packaging materials employed herein include, for example, those customarily utilized in commercial kits sold for use with nucleic acid sequencing systems. Exemplary packaging materials include, without limitation, glass, plastic, paper, foil, and the like, capable of holding within fixed limits a component set forth herein. The packaging material can include a label which indicates a particular use for the components. The use for the kit that is indicated by the label in some instances is one or more of the methods set forth herein as appropriate for the particular combination of components present in the kit. For example, a label in some instances indicates that the kit is useful for a method of detecting mutations in a nucleic acid sample using the PTA method. Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like. It will be understood that not all components necessary for a particular reaction need be present in a particular kit. Rather one or more additional components in some instances are provided from other sources. The instructions provided with a kit in some instances identify the additional component(s) that are to be provided and where they can be obtained. In one embodiment, a kit provides at least one amplification primer; at least one nucleic acid polymerase; a mixture of at least two nucleotides, wherein the mixture of nucleotides comprises at least one terminator nucleotide which terminates nucleic acid replication by the polymerase; and instructions for use of the kit. In some instances, the kit provides reagents to perform the methods described herein, such as PTA. In some instances, a kit further comprises reagents configured for gene editing (e.g., Crispr/cas9 or other method described herein).

In a related aspect, the invention provides a kit comprising a reverse transcriptase, a nucleic acid polymerase, one or more amplification primers, a mixture of nucleotides comprising one or more terminator nucleotides, and optionally instructions for use. In one embodiment of the kits of the invention, the nucleic acid polymerase is a strand displacing DNA polymerase. In one embodiment of the kits of the invention, the nucleic acid polymerase is selected from bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo−) DNA polymerase, Deep Vent DNA polymerase, Deep Vent (exo−) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of the kits of the invention, the nucleic acid polymerase has 3′->5′ exonuclease activity and the terminator nucleotides inhibit such 3′->5′ exonuclease activity (e.g., nucleotides with modification to the alpha group [e.g., alpha-thio dideoxynucleotides], C3 spacer nucleotides, locked nucleic acids (LNA), inverted nucleic acids, 2′ fluoro nucleotides, 3′ phosphorylated nucleotides, 2′-O-Methyl modified nucleotides, trans nucleic acids). In one embodiment of the kits of the invention, the nucleic acid polymerase does not have 3′->5′ exonuclease activity (e.g., Bst DNA polymerase, exo(−) Bst polymerase, exo(−) Bca DNA polymerase, Bsu DNA polymerase, Vent_{R} (exo−) DNA polymerase, Deep Vent (exo−) DNA polymerase, Klenow Fragment (exo−) DNA polymerase, Therminator DNA polymerase). In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

Numbered Embodiments

Described herein are the following numbered embodiments 1-104. 1. Provided herein is a method of determining a mutations comprising: a. exposing a population of cells to a gene editing method, wherein the gene editing method utilizes reagents configured to effect a mutation in a target sequence; b. isolating single cells from the population; c. providing a cell lysate from a single cell; d. contacting the cell lysate with at least one amplification primer, at least one nucleic acid polymerase, and a mixture of nucleotides, wherein the mixture of nucleotides comprises at least one terminator nucleotide which terminates nucleic acid replication by the polymerase, and e. amplifying the target nucleic acid molecule to generate a plurality of terminated amplification products, wherein the replication proceeds by strand displacement replication; f. ligating the molecules obtained in step (e) to adaptors, thereby generating a library of amplification products; g. sequencing the library of amplification products, and h. comparing the sequences of amplification products to at least one reference sequence to identify at least one mutation. 2. Further provided herein is a method of embodiment 1, wherein the at least one mutation is present in the target sequence. 3. Further provided herein is a method of embodiment 1, wherein the at least one mutation is not present in the target sequence. 4. Further provided herein is a method of embodiment 1 or 2, wherein the gene editing method comprising use of CRISPR, TALEN, ZFN, recombinase, or meganucleases. 5. Further provided herein is a method of embodiment 1 or 2, wherein the gene editing technique comprises use of CRISPR. 6. Further provided herein is a method of embodiment 1 or 2, wherein the gene editing technique comprises use of a gene therapy method. 7. Further provided herein is a method of embodiment 6, wherein gene therapy method is not configured to modify somatic or germline DNA of a cell. 8. Further provided herein is a method of embodiment 5, wherein the reference sequence is a genome. 9. Further provided herein is a method of embodiment 5, wherein the reference sequence is a specificity-determining sequence, where in the specificity-determining sequence is configured to bind to the target sequence. 10. Further provided herein is a method of embodiment 9, wherein the at least one mutation is present in a region of a sequence differing from the specificity-determining sequence by at least 1 bases. 11. Further provided herein is a method of embodiment 9, wherein the at least one mutation is present in a region of a sequence differing from the specificity-determining sequence by at least 2 bases. 12. Further provided herein is a method of embodiment 9, wherein the at least one mutation is present in a region of a sequence differing from the specificity-determining sequence by at least 3 bases. 13. Further provided herein is a method of embodiment 9, wherein the at least one mutation is present in a region of a sequence differing from the specificity-determining sequence by at least 5 bases. 14. Further provided herein is a method of embodiment 1, wherein the at least one mutation comprises an insertion, deletion, or substitution. 15. Further provided herein is a method of embodiment 5, wherein the reference sequence is the sequence of a CRISPR RNA (crRNA). 16. Further provided herein is a method of embodiment 5, wherein the reference sequence is the sequence of a single guide RNA (sgRNA). 17. Further provided herein is a method of embodiment 5, wherein the at least one mutation is present in a region of a sequence which binds to catalytically active Cas9. 18. Further provided herein is a method of embodiment 1, wherein the single cell is a mammalian cell. 19. Further provided herein is a method of embodiment 1, wherein the single cell is a human cell. 20. Further provided herein is a method of any one of embodiments 1-19, wherein the single cells originate from liver, skin, kidney, blood, or lung. 21. Further provided herein is a method of any one of embodiments 1-20, wherein the single cells is a primary cell. 22. Further provided herein is a method of any one of embodiments 1-20, wherein the single cells is a stem cell. 23. Further provided herein is a method of any one of embodiments 1-20, wherein at least some of the amplification products comprise a barcode. 24. Further provided herein is a method of any one of embodiments 1-20, wherein at least some of the amplification products comprise at least two barcodes. 25. Further provided herein is a method of embodiment 23, wherein the barcode comprises a cell barcode. 26. Further provided herein is a method of embodiment 23 or 25, wherein the barcode comprises a sample barcode. 27. Further provided herein is a method of any one of embodiments 1-26, wherein at least some of the amplification primers comprise a unique molecular identifier (UMI). 28. Further provided herein is a method of any one of embodiments 1-26, wherein at least some of the amplification primers comprise at least two unique molecular identifiers (UMIs). 29. Further provided herein is a method of any one of embodiments 1-27, wherein the method further comprises an additional amplification step using PCR. 30. Further provided herein is a method of any one of embodiments 1-29, wherein the method further comprises removing at least one terminator nucleotide from the terminated amplification products prior to ligation to adapters. 31. Further provided herein is a method of any one of embodiments 1-30, wherein single cells are isolated from the population using a method comprises a microfluidic device. 32. Further provided herein is a method of any one of embodiments 1-31, wherein the at least one mutation occurs in less than 50% of the population of cells. 33. Further provided herein is a method of any one of embodiments 1-31, wherein the at least one mutation occurs in less than 25% of the population of cells. 34. Further provided herein is a method of any one of embodiments 1-31, wherein the at least one mutation occurs in less than 1% of the population of cells. 35. Further provided herein is a method of any one of embodiments 1-31, wherein the at least one mutation occurs in no more than 0.1% of the population of cells. 36. Further provided herein is a method of any one of embodiments 1-31, wherein the at least one mutation occurs in no more than 0.01% of the population of cells. 37. Further provided herein is a method of any one of embodiments 1-31, wherein the at least one mutation occurs in no more than 0.001% of the population of cells. 38. Further provided herein is a method of any one of embodiments 1-31, wherein the at least one mutation occurs in no more than 0.0001% of the population of cells. 39. Further provided herein is a method of any one of embodiments 1-31, wherein the at least one mutation occurs in no more than 25% of the amplification product sequences. 40. Further provided herein is a method of any one of embodiments 1-31, wherein the at least one mutation occurs in no more than 1% of the amplification product sequences. 41. Further provided herein is a method of any one of embodiments 1-31, wherein the at least one mutation occurs in no more than 0.1% of the amplification product sequences. 42. Further provided herein is a method of any one of embodiments 1-31, wherein the at least one mutation occurs in no more than 0.01% of the amplification product sequences. 43. Further provided herein is a method of any one of embodiments 1-31, wherein the at least one mutation occurs in no more than 0.001% of the amplification product sequences. 44. Further provided herein is a method of any one of embodiments 1-31, wherein the at least one mutation occurs in no more than 0.0001% of the amplification product sequences. 45. Further provided herein is a method of any one of embodiments 1-31, wherein the at least one mutation is present in a region of a sequence correlated with a genetic disease or condition. 46. Further provided herein is a method of any one of embodiments 1-31, wherein the at least one mutation is present in a region of a sequence not correlated with binding of a DNA repair enzyme. 47. Further provided herein is a method of any one of embodiments 1-31, wherein the at least one mutation is present in a region of a sequence not correlated with binding of MRE11. 48. Further provided herein is a method of any one of embodiments 1-31, wherein the method further comprises identifying a false positive mutation previously sequenced by an alternative off-target detection method. 49. Further provided herein is a method of embodiment 48, wherein the off-target detection method is in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq. 50. Provided herein is a method of identifying specificity-determining sequences comprising: a. providing a library of nucleic acids, wherein at least some of the nucleic acids comprise a specificity-determining sequence; b. performing a gene editing method on at least one cell, wherein the gene editing method comprises contacting the cell with reagents comprising at least one specificity-determining sequence; c. sequencing a genome of the at least one cell using Further provided herein is a method of any one of embodiments 1-38, wherein the specificity-determining sequence contacted with the at least one cell is identified; and d. identifying at least one specificity-determining sequence which provides the fewest off-target mutations. 51. Further provided herein is a method of embodiment 50, wherein the off-target mutations are silent mutations. 52. Further provided herein is a method of embodiment 50, wherein the off-target mutations are present outside of gene coding regions. 53. Provided herein is a method of in-vivo mutational analysis comprising: a. performing a gene editing method on at least one cell in a living organism, wherein the gene editing method comprises contacting the cell with reagents comprising at least one specificity-determining sequence; b. isolating at least one cell from the organism; c. sequencing a genome of the at least one cell using Further provided herein is a method of any one of embodiments 1-49. 54. Further provided herein is a method of embodiment 53, wherein the method comprises at least two cells. 55. Further provided herein is a method of embodiment 54, further comprising identifying mutations by comparing the genome of a first cell with the genome of a second cell. 56. Further provided herein is a method of embodiment 54 or 55, wherein the first cell and the second cell are from different tissues. 57. Provided herein are a method of predicting the age of a subject comprising: a. providing at least one sample from the subject, wherein the at least one sample comprises a genome; b. sequencing a genome using Further provided herein is a method of any one of embodiments 1-38 to identify mutations; c. comparing mutations obtained in step b with a standard reference curve, wherein the standard reference curve correlates mutation count and location with a verified age; and d. predicting the age of the subject based on the mutation comparison to the standard reference curve. 58. Further provided herein is a method of embodiment 57, wherein the standard reference curve is specific for a subject's sex. 59. Further provided herein is a method of embodiment 57, wherein the standard reference curve is specific for a subject's ethnicity. 60. Further provided herein is a method of embodiment 57, wherein the standard reference curve is specific for a subject's geographic location where the subject spent a period of the subject's life. 61. Further provided herein is a method of any one of embodiments 57-60, wherein the subject is less than 50 years old. 62. Further provided herein is a method of any one of embodiments 57-60, wherein the subject is less than 18 years old. 63. Further provided herein is a method of any one of embodiments 57-60, wherein the subject is less than 15 years old. 64. Further provided herein is a method of any one of embodiments 57-63, wherein the at least one sample is more than 10 years old. 65. Further provided herein is a method of any one of embodiments 57-63, wherein the at least one sample is more than 100 years old. 66. Further provided herein is a method of any one of embodiments 57-63, wherein the at least one sample is more than 1000 years old. 67. Further provided herein is a method of any one of embodiments 57-66, wherein at least 2 samples are sequenced. 68. Further provided herein is a method of any one of embodiments 57-66, wherein at least 5 samples are sequenced. 69. Further provided herein is a method of embodiment 67, wherein the at least two samples are from different tissues. 70. Provided herein is a method for sequencing a microbial or viral genome comprising: a. obtaining a sample comprising one or more genomes or genome fragments; b. sequencing the sample using Further provided herein is a method of any one of embodiments 1-38 to obtain a plurality of sequencing reads; and c. assembling and sorting the sequencing reads to generate the microbial or viral genome. 71. Further provided herein is a method of embodiment 70, wherein the sample comprises genomes from at least two organisms. 72. Further provided herein is a method of embodiment 70, wherein the sample comprises genomes from at least ten organisms. 73. Further provided herein is a method of embodiment 70, wherein the sample comprises genomes from at least 100 organisms. 74. Further provided herein is a method of any one of embodiments 70-73, wherein the sample origin is an environment comprising deep sea vents, ocean, mines, streams, lakes, meteorites, glaciers, or volcanoes. 75. Further provided herein is a method of any one of embodiments 70-74, further comprising identifying at least one gene in the microbial genome. 76. Further provided herein is a method of any one of embodiments 70-75, wherein the microbial genome corresponds to an unculturable organism. 77. Further provided herein is a method of embodiment 76, wherein the microbial genome corresponds to an symbiotic organism. 78. Further provided herein is a method of any one of embodiments 70-77, further comprising cloning of the at least one gene in a recombinant host organism. 79. Further provided herein is a method of embodiment 78, wherein the recombinant host organism is a bacteria. 80. Further provided herein is a method of embodiment 79, wherein the recombinant host organism is Escherichia, Bacillus, or Streptomyces. 81. Further provided herein is a method of embodiment 78, wherein the recombinant host organism is a eukaryotic cell. 82. Further provided herein is a method of embodiment 81, wherein the recombinant host organism is a yeast cell. 83. Further provided herein is a method of embodiment 82, wherein the recombinant host organism is Saccharomyces or Pichia. 84. Provided herein is a kit for nucleic acid sequencing comprising: a. at least one amplification primer; b. at least one nucleic acid polymerase; c. a mixture of at least two nucleotides, wherein the mixture of nucleotides comprises at least one terminator nucleotide which terminates nucleic acid replication by the polymerase; and d. instructions for use of the kit to perform nucleic acid sequencing. 85. Further provided herein is a kit of embodiment 84, wherein the at least one amplification primer is a random primer. 86. Further provided herein is a kit of embodiment 84, wherein the nucleic acid polymerase is a DNA polymerase. 87. Further provided herein is a kit of embodiment 86, wherein the DNA polymerase is a strand displacing DNA polymerase. 88. Further provided herein is a kit of any one of embodiments 84-87, wherein the nucleic acid polymerase is bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, VentR DNA polymerase, VentR (exo−) DNA polymerase, Deep Vent DNA polymerase, Deep Vent (exo−) DNA polymerase, IsoPol DNA polymerase, DNA polymerase I, Therminator DNA polymerase, T5 DNA polymerase, Sequenase, T7 DNA polymerase, T7-Sequenase, or T4 DNA polymerase. 89. Further provided herein is a kit of any one of embodiments 84-88, wherein the nucleic acid polymerase comprises 3′->5′ exonuclease activity and the at least one terminator nucleotide inhibits the 3′->5′ exonuclease activity. 90. Further provided herein is a kit of any one of embodiments 84-88, wherein the nucleic acid polymerase does not comprise 3′->5′ exonuclease activity. 91. Further provided herein is a kit of any one of embodiments 84-88, wherein the polymerase is Bst DNA polymerase, exo(−) Bst polymerase, exo(−) Bca DNA polymerase, Bsu DNA polymerase, VentR (exo−) DNA polymerase, Deep Vent (exo−) DNA polymerase, Klenow Fragment (exo−) DNA polymerase, or Therminator DNA polymerase. 92. Further provided herein is a kit of any one of embodiments 84-91, wherein the least one terminator nucleotide comprises modifications of the r group of the 3′ carbon of the deoxyribose. 93. Further provided herein is a kit of any one of embodiments 84-92, wherein the at least one terminator nucleotide is selected from the group consisting of 3′ blocked reversible terminator containing nucleotides, 3′ unblocked reversible terminator containing nucleotides, terminators containing 2′ modifications of deoxynucleotides, terminators containing modifications to the nitrogenous base of deoxynucleotides, and combinations thereof. 94. Further provided herein is a kit of any one of embodiments 84-93, wherein the at least one terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. 95. Further provided herein is a kit of any one of embodiments 84-94, wherein the at least one terminator nucleotide are selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. 96. Further provided herein is a kit of any one of embodiments 84-95, wherein the nucleotides with modification to the alpha group are alpha-thio dideoxynucleotides. 97. Further provided herein is a kit of any one of embodiments 84-96, wherein the amplification primers are 4 to 70 nucleotides in length. 98. Further provided herein is a kit of any one of embodiments 84-97, wherein the at least one amplification primer is 4 to 20 nucleotides in length. 99. Further provided herein is a kit of any one of embodiments 84-98, wherein the at least one amplification primer comprises a randomized region. 100. Further provided herein is a kit of embodiment 99, wherein the randomized region is 4 to 20 nucleotides in length. 101. Further provided herein is a kit of embodiment 99 or 100, wherein the randomized region is 8 to 15 nucleotides in length. 102. Further provided herein is a kit of any one of embodiments 84-101, wherein the kit further comprises a library preparation kit. 103. Further provided herein is a kit of embodiment 102, wherein the library preparation kit comprises one or more of: a. at least one polynucleotide adapter; b. at least one high-fidelity polymerase; c. at least one ligase; d. a reagent for nucleic acid shearing; and e. at least one primer, wherein the primer is configured to bind to the adapter. 104. Further provided herein is a kit of any one of embodiments 84-103, wherein the kit further comprises reagents configured for gene editing.

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1

### Primary Template-Directed Amplification (PTA)

While PTA can be used for any nucleic acid amplification, it is particularly useful for whole genome amplification as it allows to capture a larger percentage of a cell genome in a more uniform and reproducible manner and with lower error rates than the currently used methods such as, e.g., Multiple Displacement Amplification (MDA), avoiding such drawbacks of the currently used methods as exponential amplification at locations where the polymerase first extends the random primers which results in random overrepresentation of loci and alleles and mutation propagation (see FIGS. 1A-1C).

Cell Culture

Human NA12878 (Coriell Institute) cells were maintained in RPMI media, supplemented with 15% FBS and 2 mM L-glutamine, and 100 units/mL of penicillin, 100 μg/mL of streptomycin, and 0.25 μg/mL of Amphotericin B (Gibco, Life Technologies). The cells were seeded at a density of 3.5×10⁵ cells/ml. The cultures were split every 3 days and were maintained in a humidified incubator at 37 C with 5% CO₂.

Single-Cell Isolation and WGA

After culturing NA12878 cells for a minimum of three days after seeding at a density of 3.5×10⁵ cells/ml, 3 mL of cell suspension were pelleted at 300×g for 10 minutes. The medium was then discarded and the cells were washed three times with 1 mL of cell wash buffer (1× PBS containing 2% FBS without Mg² or Ca²) being spun at 300×g, 200×g and finally 100×g for 5 minutes. The cells were then resuspended in 500 μL of cell wash buffer. This was followed by staining with 100 nM of Calcein AM (Molecular Probes) and 100 ng/ml of propidium iodide (PI; Sigma-Aldrich) to distinguish the live cell population. The cells were loaded on a BD FACScan flow cytometer (FACSAria II) (BD Biosciences) that had been thoroughly cleaned with ELIMINase (Decon Labs) and calibrated using Accudrop fluorescent beads (BD Biosciences) for cell sorting. A single cell from the Calcein AM-positive, PI-negative fraction was sorted in each well of a 96 well plate containing 3 μL of PBS with 0.2% Tween 20 in the cells that would undergo PTA (Sigma-Aldrich). Multiple wells were intentionally left empty to be used as no template controls (NTC). Immediately after sorting, the plates were briefly centrifuged and placed on ice. Cells were then frozen at a minimum of overnight at −20° C. On a subsequent day, WGA Reactions were assembled on a pre-PCR workstation that provides a constant positive pressure of HEPA filtered air and which was decontaminated with UV light for 30 minutes before each experiment.

MDA was carried out using with modifications that have previously been shown to improve the amplification uniformity. Specifically, exonuclease-resistant random primers were added to a lysis buffer/mix to a final concentration of 125 μM. 4 μL of the resulting lysis/denaturing mix was added to the tubes containing the single cells, vortexed, briefly spun and incubated on ice for 10 minutes. The cell lysates were neutralized by adding 3 μL of a quenching buffer, mixed by vortexing, centrifuged briefly, and placed at room temperature. This was followed by addition of 40 μl of amplification mix before incubation at 30° C. for 8 hours after which the amplification was terminated by heating to 65° C. for 3 minutes.

PTA was carried out by first further lysing the cells after freeze thawing by adding 2 μl a prechilled solution of a 1:1 mixture of 5% Triton X-100 (Sigma-Aldrich) and 20 mg/ml Proteinase K (Promega). The cells were then vortexed and briefly centrifuged before placing at 40 degrees for 10 minutes. 4 μl of lysis buffer/mix and 1 μl of 500 μM exonuclease-resistant random primer were then added to the lysed cells to denature the DNA prior to vortexing, spinning, and placing at 65 degrees for 15 minutes. 4 μl of room temperature quenching buffer was then added and the samples were vortexed and spun down. 56 μl of amplification mix (primers, dNTPs, polymerase, buffer) that contained alpha-thio-ddNTPs at equal ratios at a concentration of 1200 μM in the final amplification reaction. The samples were then placed at 30° C. for 8 hours after which the amplification was terminated by heating to 65° C. for 3 minutes.

After the amplification step, the DNA from both MDA and PTA reactions were purified using AMPure XP magnetic beads (Beckman Coulter) at a 2:1 ratio of beads to sample and the yield was measured using the Qubit dsDNA HS Assay Kit with a Qubit 3.0 fluorometer according to the manufacturer's instructions (Life Technologies).

Library Preparation

The MDA reactions resulted in the production of 40 μg of amplified DNA. 1 μg of product was enzymatically fragmented for 30 minutes following standard procedures. The samples then underwent standard library preparation with 15 μM of dual index adapters (end repair by a T4 polymerase, T4 polynucleotide kinase, and Taq polymerase for A-tailing) and 4 cycles of PCR. Each PTA reaction generated between 40-60 ng of material which was used for standard DNA sequencing library preparation. 2.5 μM adapters with UMIs and dual indices were used in the ligation with T4 ligase, and 15 cycles of PCR (hot start polymerase) were used in the final amplification. The libraries were then cleaned up using a double sided SPRI using ratios of 0.65× and 0.55× for the right and left sided selection, respectively. The final libraries were quantified using the Qubit dsDNA BR Assay Kit and 2100 Bioanalyzer (Agilent Technologies) before sequencing on the Illumina NextSeq platform. All Illumina sequencing platforms, including the NovaSeq, are also compatible with the protocol.

Data Analysis

Sequencing reads were demultiplexed based on cell barcode using Bcl2fastq. The reads were then trimmed using trimmomatic, which was followed by alignment to hg19 using BWA. Reads underwent duplicate marking by Picard, followed by local realignment and base recalibration using GATK 4.0. All files used to calculate quality metrics were downsampled to twenty million reads using Picard DownSampleSam. Quality metrics were acquired from the final bam file using qualimap, as well as Picard AlignmentSummaryMetrics and CollectWgsMetrics. Total genome coverage was also estimated using Preseq.

Variant Calling

Single nucleotide variants and Indels were called using the GATK UnifiedGenotyper from GATK 4.0. Standard filtering criteria using the GATK best practices were used for all steps in the process (https://software.broadinstitute.org/gatk/best-practices/). Copy number variants were called using Control-FREEC (Boeva et al., Bioinformatics, 2012, 28(3):423-5). Structural variants were also detected using CREST (Wang et al., Nat Methods, 2011, 8(8):652-4).

Results

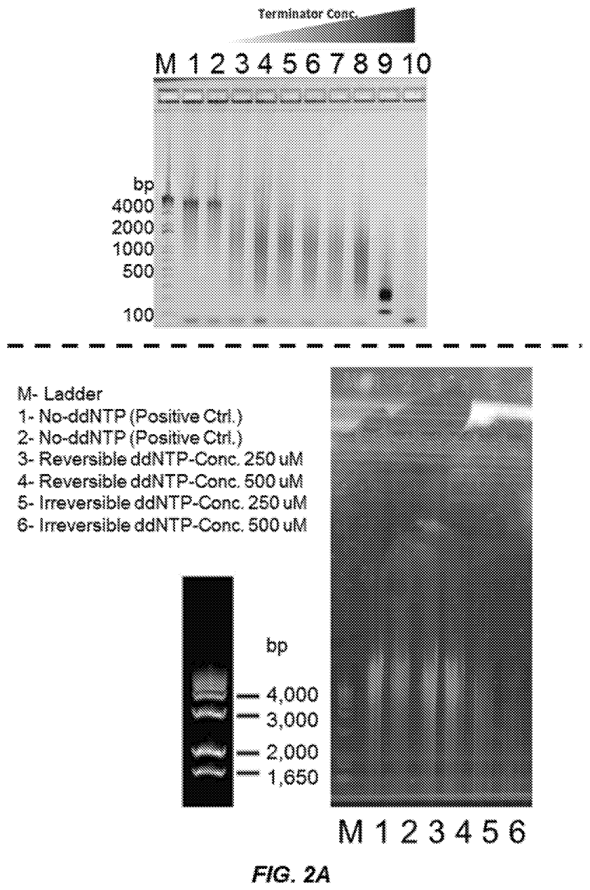

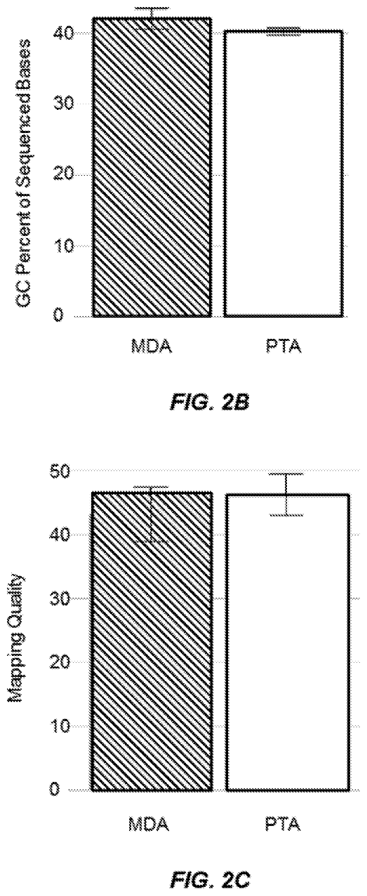

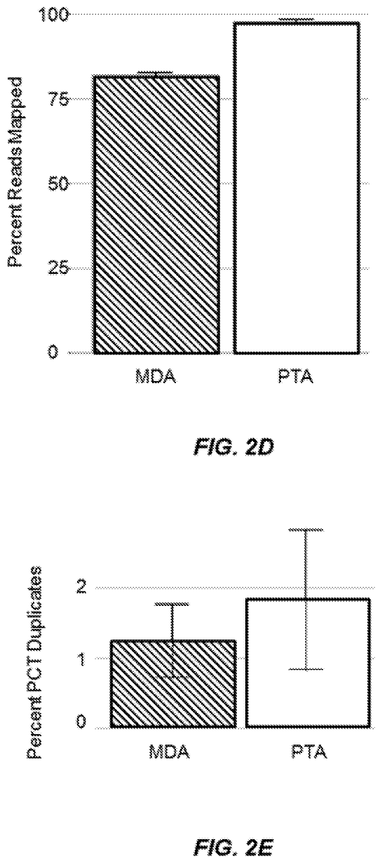

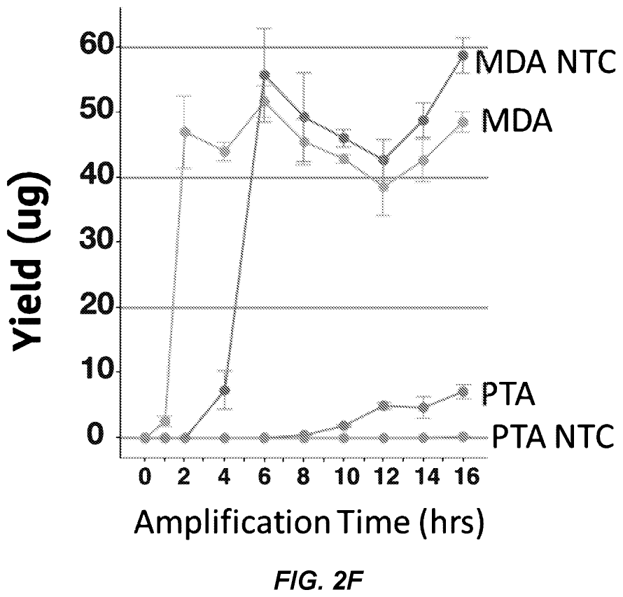

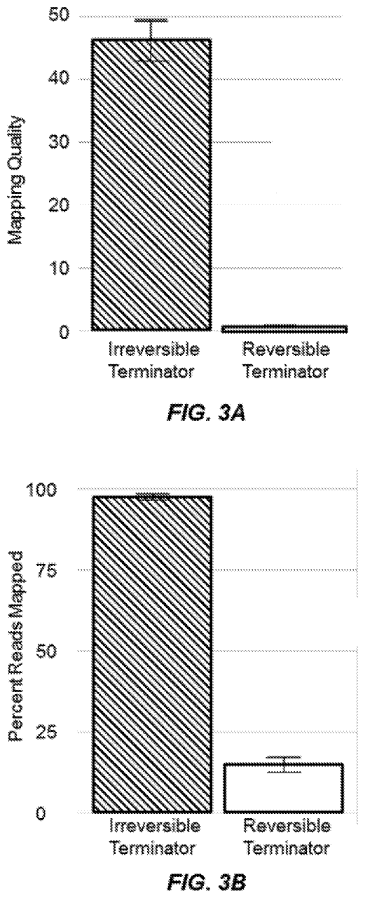

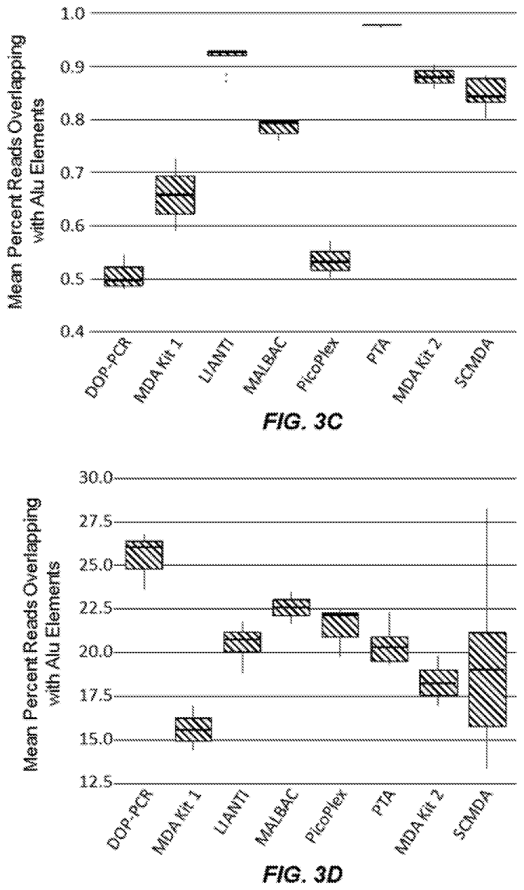

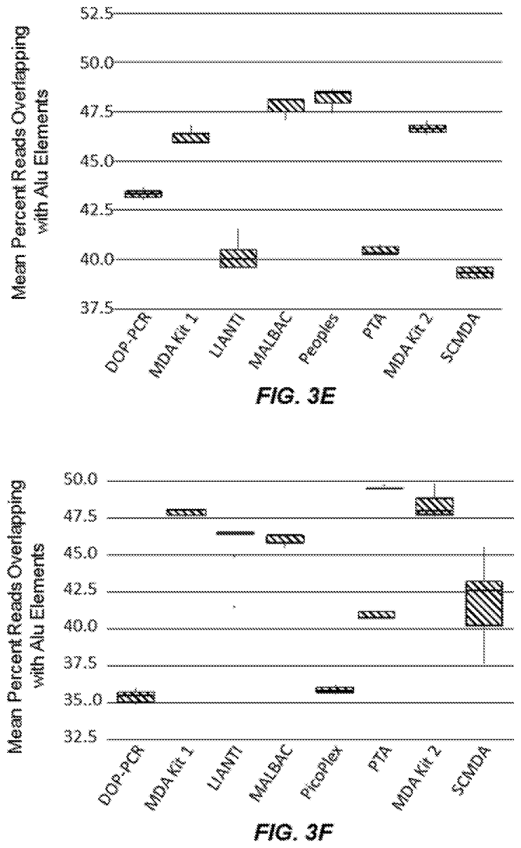

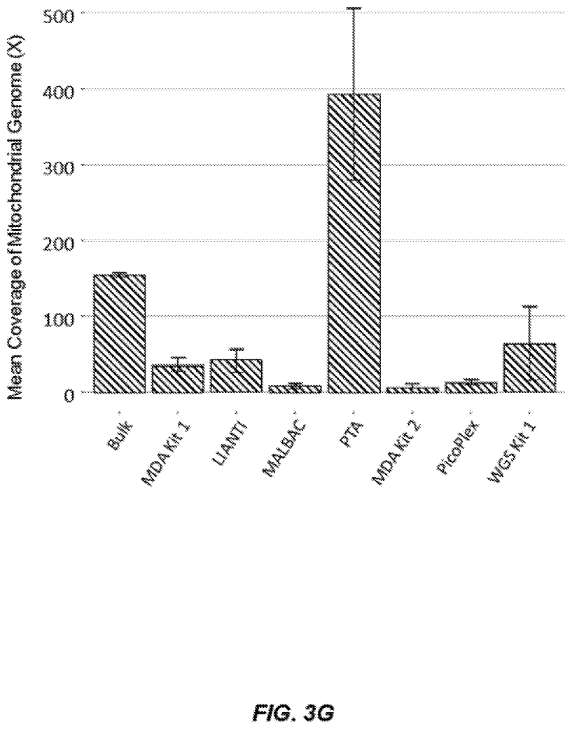

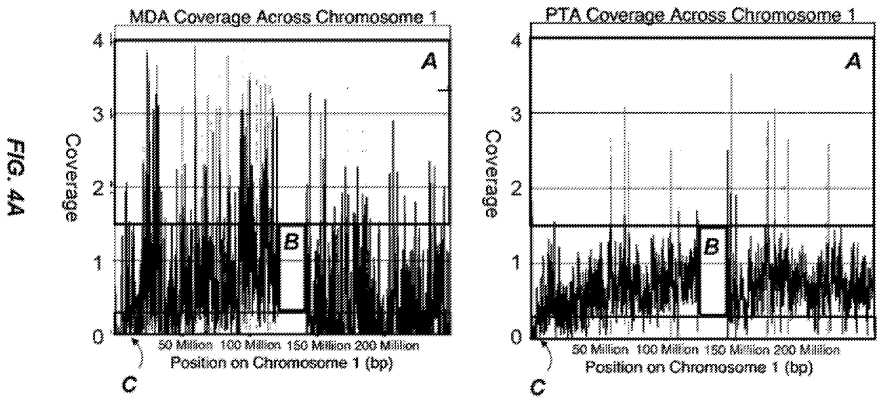

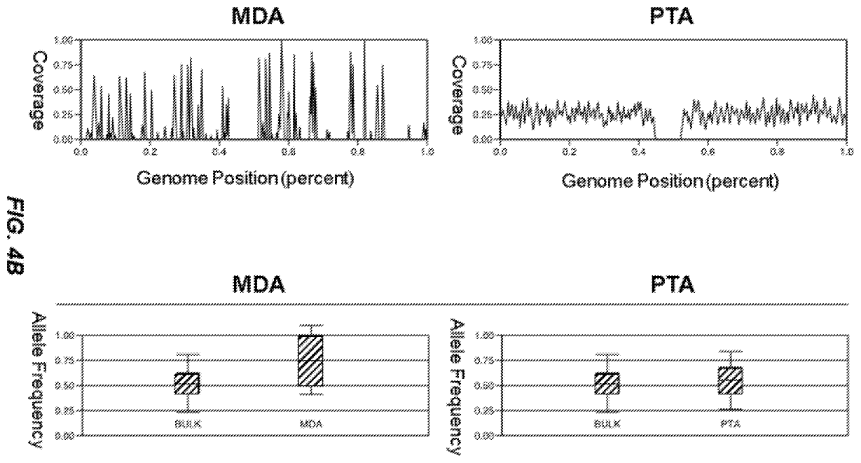

As shown in FIG. 3A and FIG. 3B, the mapping rates and mapping quality scores of the amplification with dideoxynucleotides (“reversible”) alone are 15.0+/−2.2 and 0.8+/−0.08, respectively, while the incorporation of exonuclease-resistant alpha-thio dideoxynucleotide terminators (“irreversible”) results in mapping rates and quality scores of 97.9+/−0.62 and 46.3+/−3.18, respectively. Experiments were also run using a reversible ddNTP, and different concentrations of terminators. (FIG. 2A, bottom)

FIGS. 2B-2E show the comparative data produced from NA12878 human single cells that underwent MDA (following the method of Dong, X. et al., Nat Methods. 2017, 14(5):491-493) or PTA. While both protocols produced comparable low PCR duplication rates (MDA 1.26%+/−0.52 vs PTA 1.84%+/−0.99). and GC % (MDA 42.0+/−1.47 vs PTA 40.33+/−0.45), PTA produced smaller amplicon sizes. The percent of reads that mapped and mapping quality scores were also significantly higher for PTA as compared to MDA (PTA 97.9+/−0.62 vs MDA 82.13+/−0.62 and PTA 46.3+/−3.18 vs MDA 43.2+/−4.21, respectively). Overall, PTA produces more usable, mapped data when compared to MDA. FIG. 4A shows that, as compared to MDA, PTA has significantly improved uniformity of amplification with greater coverage breadth and fewer regions where coverage falls to near 0. The use of PTA allows identifying low frequency sequence variants in a population of nucleic acids, including variants which constitute ≥0.01% of the total sequences. PTA can be successfully used for single cell genome amplification.

### Example 2

### Comparative Analysis of PTA

Benchmarking PTA and SCMDA Cell Maintenance and Isolation

Lymphoblastoid cells from 1000 Genome Project subject NA12878 (Coriell Institute, Camden, N.J., USA) were maintained in RPMI media, which was supplemented with 15% FBS, 2 mM L-glutamine, 100 units/mL of penicillin, 100 μg/mL of streptomycin, and 0.25 μg/mL of Amphotericin B). The cells were seeded at a density of 3.5×10⁵ cells/ml and split every 3 days. They were maintained in a humidified incubator at 37° C. with 5% CO₂. Prior to single cell isolation, 3 mL of suspension of cells that had expanded over the previous 3 days was spun at 300×g for 10 minutes. The pelleted cells were washed three times with 1 mL of cell wash buffer (1× PBS containing 2% FBS without Mg²⁺ or Ca²⁺)) where they were spun sequentially at 300×g, 200×g, and finally 100×g for 5 minutes to remove dead cells. The cells were then resuspended in 500 uL of cell wash buffer, which was followed by staining with 100 nM of Calcein AM and 100 ng/ml of propidium iodide (PI) to distinguish the live cell population. The cells were loaded on a BD FACScan flow cytometer (FACSAria II) that had been thoroughly cleaned with ELIMINase and calibrated using Accudrop fluorescent beads. A single cell from the Calcein AM-positive, PI-negative fraction was sorted in each well of a 96 well plate containing 3 uL of PBS with 0.2% Tween 20. Multiple wells were intentionally left empty to be used as no template controls. Immediately after sorting, the plates were briefly centrifuged and placed on ice. Cells were then frozen at a minimum of overnight at −80° C.

PTA and SCMDA Experiments

WGA Reactions were assembled on a pre-PCR workstation that provides constant positive pressure with HEPA filtered air and which was decontaminated with UV light for 30 minutes before each experiment. MDA was carried according to the SCMDA according to the published protocol (Dong et al. Nat. Meth. 2017, 14, 491-493). Specifically, exonuclease-resistant random primers were added at a final concentration of 12.5 uM to the lysis buffer. 4 uL of the resulting lysis mix was added to the tubes containing the single cells, pipetted three times to mix, briefly spun and incubated on ice for 10 minutes. The cell lysates were neutralized by adding 3 uL of quenching buffer, mixed by pipetting 3 times, centrifuged briefly, and placed on ice. This was followed by addition of 40 uL of amplification mix before incubation at 30° C. for 8 hours after which the amplification was terminated by heating to 65° C. for 3 minutes. PTA was carried out by first further lysing the cells after freeze thawing by adding 2 μL of a prechilled solution of a 1:1 mixture of 5% Triton X-100 and 20 mg/ml Proteinase K. The cells were then vortexed and briefly centrifuged before placing at 40 degrees for 10 minutes. 4 μL of denaturing buffer and 1 μl of 500 μM exonuclease-resistant random primer were then added to the lysed cells to denature the DNA prior to vortexing, spinning, and placing at 65° C. for 15 minutes. 4 μL of room temperature quenching solution was then added and the samples were vortexed and spun down. 56 μL of amplification mix that contained alpha-thio-ddNTPs at equal ratios at a concentration of 1200 μM in the final amplification reaction. The samples were then placed at 30° C. for 8 hours after which the amplification was terminated by heating to 65° C. for 3 minutes. After the SCMDA or PTA amplification, the DNA was purified using AMPure XP magnetic beads at a 2:1 ratio of beads to sample and the yield was measured using the Qubit dsDNA HS Assay Kit with a Qubit 3.0 fluorometer according to the manufacturer's instructions. PTA experiments were also run using reversible ddNTPs, and different concentrations of terminators. (FIG. 2A, top)

Library Preparation

1 ug of SCMDA product was enzymatically fragmented for 30 minutes according to standard protocols. The samples then underwent standard library preparation with 15 uM of unique dual index adapters and 4 cycles of PCR. The entire product of each PTA reaction was used for DNA sequencing library preparation, without fragmentation. 2.5 uM of unique dual index adapter was used in the ligation, and 15 cycles of PCR were used in the final amplification. The libraries from SCMDA and PTA were then visualized on a 1% Agarose E-Gel. Fragments between 400-700 bp were excised from the gel and recovered using a Gel DNA Recovery Kit. The final libraries were quantified using the Qubit dsDNA BR Assay Kit and Agilent 2100 Bioanalyzer before sequencing on the NovaSeq 6000.

Data Analysis

Data was trimmed using trimmomatic, which was followed by alignment to hg19 using BWA. Reads underwent duplicate marking by Picard, followed by local realignment and base recalibration using GATK 3.5 best practices. All files were downsampled to the specified number of reads using Picard DownSampleSam. Quality metrics were acquired from the final bam file using qualimap, as well as Picard AlignmentMetricsAummary and CollectWgsMetrics. Lorenz curves were drawn and Gini Indices calculated using htSeqTools. SNV calling was performed using UnifiedGenotyper, which were then filtered using the standard recommended criteria (QD<2.0∥FS>60.0∥MQ<40.0∥SOR>4.0∥MQRankSum<−12.5∥ReadPosRankSum<−8.0). No regions were excluded from the analyses and no other data normalization or manipulations were performed. Sequencing metrics for the methods tested are found in Table 1.

TABLE 1
Comparison of sequencing metrics between methods tested.

| | | MDA | | | | MDA | DOP |
|---|---|---|---|---|---|---|---|
| | PTA | Kit 2 | PicoPlex | MALBAC | LIANTI | Kit 1 | PCR |
| % | 97 | 88 | 55 | 79 | 92 | 65 | 52 |
| Genome | | | | | | | |
| Mapping | | | | | | | |
| % | 95 | 75 | 43 | 60 | 82 | 73 | 23 |
| Genome | | | | | | | |
| Recovery | | | | | | | |
| (300M | | | | | | | |
| reads) | | | | | | | |
| CV of | 0.8 | 1.8 | 3 | 2.5 | 1.1 | 2 | 3.5 |
| Coverage | | | | | | | |
| (300M | | | | | | | |
| reads) | | | | | | | |
| SNV | 76 | 50 | 15 | 34 | 49 | 46 | 5 |
| Sensitivity | | | | | | | |
| % (300M | | | | | | | |
| reads) | | | | | | | |
| SNV | 93 | 91 | 56 | 47 | 88 | 90 | 35 |
| Precision | | | | | | | |
| % (300M | | | | | | | |
| reads) | | | | | | | |

CV = Coefficient of Variation;
SNV = Single Nucleotide Variation;
values refer to 15X coverage.

Genome Coverage Breadth and Uniformity

Comprehensive comparisons of PTA to all common single-cell WGA methods were performed. To accomplish this, PTA and an improved version of MDA called single-cell MDA (Dong et al. Nat. Meth. 2017, 14, 491-493) (SCMDA) was performed on 10 NA12878 cells each. In addition, those results to cells that had undergone amplification with DOP-PCR (Zhang et al. PNAS 1992, 89, 5847-5851), MDA Kit 1 (Dean et al. PNAS 2002, 99, 5261-5266), MDA Kit 2, MALBAC (Zong et al. Science 2012, 338, 1622-1626), LIANTI (Chen et al., Science 2017, 356, 189-194), or PicoPlex (Langmore, Pharmacogenomics 3, 557-560 (2002)) was compared using data produced as part of the LIANTI study.

To normalize across samples, raw data from all samples were aligned and underwent pre-processing for variant calling using the same pipeline. The bam files were then subsampled to 300 million reads each prior to performing comparisons. Importantly, the PTA and SCMDA products were not screened prior to performing further analyses while all other methods underwent screening for genome coverage and uniformity before selecting the highest quality cells that were used in subsequent analyses. Of note, SCMDA and PTA were compared to bulk diploid NA12878 samples while all other methods were compared to bulk BJ1 diploid fibroblasts that had been used in the LIANTI study. As seen in FIGS. 3C-3F, PTA had the highest percent of reads aligned to the genome, as well as the highest mapping quality. PTA, LIANTI, and SCMDA had similar GC content, all of which were lower than the other methods. PCR duplication rates were similar across all methods. Additionally, the PTA method enabled smaller templates such as the mitochondrial genome to give higher coverage rates (similar to larger canonical chromosomes) relative to other methods tested (FIG. 3G).

Coverage breadth and uniformity of all methods was then compared. Examples of coverage plots across chromosome 1 are shown for SCMDA and PTA, where PTA is shown to have significantly improved uniformity of coverage and allele frequency (FIG. 4B. Coverage rates were then calculated for all methods using increasing number of reads. PTA approaches the two bulk samples at every depth, which is a significant improvement over all other methods (FIG. 5A). We then used two strategies to measure coverage uniformity. The first approach was to calculate the coefficient of variation of coverage at increasing sequencing depth where PTA was found to be more uniform than all other methods (FIG. 5B). The second strategy was to compute Lorenz curves for each subsampled bam file where PTA was again found to have the greatest uniformity (FIG. 5C). To measure the reproducibility of amplification uniformity, Gini Indices were calculated to estimate the difference of each amplification reaction from perfect uniformity (de Bourcy et al., PloS one 9, e105585 (2014)). PTA was again shown to be reproducibly more uniform than the other methods (FIG. 5D).

SNV Sensitivity

To determine the effects of these differences in the performance of the amplification methods on SNV calling, variant call rates for each to the corresponding bulk sample were compared at increasing sequencing depth. To estimate sensitivity, the percent of variants called in corresponding bulk samples that had been subsampled to 650 million reads that were found in each cell at each sequencing depth (FIG. 5E) were compared. Improved coverage and uniformity of PTA resulted in the detection of 45.6% more variants over MDA Kit 2, which was the next most sensitive method. An examination of sites called as heterozygous in the bulk sample showed that PTA had significantly diminished allelic skewing at those heterozygous sites (FIG. 5F). This finding supports the assertion that PTA not only has more even amplification across the genome, but also more evenly amplifies two alleles in the same cell.

SNV Precision

To estimate the Precision of mutation calls, the variants called in each single cell not found in the corresponding bulk sample were considered false positives. The lower temperature lysis of SCMDA significantly reduced the number of false positive variant calls (FIG. 5G). Methods using thermostable polymerases (MALBAC, PicoPlex, and DOP-PCR) showed further decreases in the SNV calling precision with increasing sequencing depth. Without being bound by theory, this is likely the result of the significantly increased error rate of those polymerases compared to phi29 DNA polymerase. In addition, the base change patterns seen in the false positive calls also appear to be polymerase-dependent (FIG. 5H). As seen in FIG. 5G, the model of suppressed error propagation in PTA is supported by the lower false positive SNV calling rate in PTA compared to standard MDA protocols. In addition, PTA has the lowest allele frequencies of false positive variant calls, which is again consistent with the model of suppressed error propagation with PTA (FIG. 5I).

### Example 3

### Direct Measurement of Environmental Mutagenicity (DMEM)

PTA was used to conduct a novel mutagenicity assay that provides a framework for performing high-resolution, genome wide human toxicogenomics studies. Previous studies such as the Ames test, relies on bacterial genetics to make measurements that are assumed to be representative of human cells while only providing limited information on the mutation number and patterns induced in each exposed cell. To overcome these limitations, a human mutagenesis system “direct measurement of environmental mutagenicity (DMEM)” was developed, wherein single human cells was exposed to an environmental compound, isolated as single cells, and subjected to single-cell sequencing to identify the new mutations induced in each cell.

Umbilical cord blood cells that express the stem/progenitor marker CD34 were exposed to increasing concentrations of the direct mutagen N-ethyl-N-nitrosourea (ENU). ENU is known to have a relatively low Swain-Scott substrate constant and has consequently been shown to predominantly act through a two-step SN1 mechanism that results in preferential alkylation of O4-thymine, O2-thymine, and O2-cytosine. Through limited sequencing of target genes, ENU has also been shown to have preference for T to A (A to T), T to C (A to G), and C to T (G to A) changes in mice, which significantly differs from the pattern seen in E. coli.

Isolation and Expansion of Cord Blood Cells for Mutagenicity Experiments

ENU (CAS 759-73-9) and D-mannitol (CAS 69-65-8) were put into solution at their maximal solubility. Fresh anticoagulant-treated umbilical cord blood (CB) was obtained from St. Louis Cord Blood Bank. CB was diluted 1:2 with PBS and mononuclear cells (MNCs) were isolated by density gradient centrifugation on Ficoll-Paque Plus according to manufacturer's instructions. CB MNCs expressing CD34 were then immunomagnetically selected using the human CD34 microbead kit and magnetic cell sorting (MACS) system as per the manufacturer. Cell count and viability were assessed using the Luna FL cell counter. CB CD34+ cells were seeded at a density of 2.5×10⁴ cells/mL in StemSpan SFEM supplemented with 1× CD34+ Expansion supplement, 100 units/mL of penicillin, and 100 ug/mL of streptomycin where they expanded for 96 hours before proceeding to mutagen exposure.

Direct Measurement of Environmental Mutagenicity (DMEM)

Expanded cord blood CD34+ cells were cultured in StemSpan SFEM supplemented with 1× CD34+ Expansion Supplement, 100 units/mL of penicillin, and 100 ug/mL of streptomycin. The cells were exposed to ENU at concentrations of 8.54, 85.4, and 854 uM, D-mannitol at 1152.8, and 11528 uM, or 0.9% sodium chloride (vehicle control) for 40 hours. Single-cell suspensions from drug-treated cells and vehicle control samples were harvested and stained for viability as described above. Single cell sorts were carried out as described above. PTA was performed and libraries were prepared using a simplified and improved protocol as per the general methods of the methods described herein, and Example 2.

Analysis of DMEM Data

Data acquired from cells in the DMEM experiments were trimmed using Trimmomatic, aligned to GRCh38 using BWA, and further processed using GATK 4.0.1 best practices without deviation from the recommended parameters. Genotyping was performed using HaplotypeCaller where joint genotypes were again filtered using standard parameters. A variant was only considered to be the result of the mutagen if it had a Phred quality score of at least 100 and was only found in one cell while not being found in the bulk sample. The trinucleotide context of each SNV was determined by extracting the surrounding bases from the reference genome using bedtools. Mutation counts and context were visualized using ggplot2 and heatmap2 in R.

To determine whether mutations were enriched in DNase I hypersensitivity sites (DHS) in CD34+ cells, the proportion of SNVs in each sample that overlap with DHS sites from 10 CD34+ primary cell datasets produced by the Roadmap Epigenomics Project were calculated. DHS sites were extended by 2 nucleosomes, or 340 bases in either direction. Each DHS dataset was paired with a single cell sample where we determined the proportion of the human genome with at least 10× coverage in that cell which overlapped with a DHS, which was compared to the proportion of SNVs that were found within the covered DHS sites.

Results

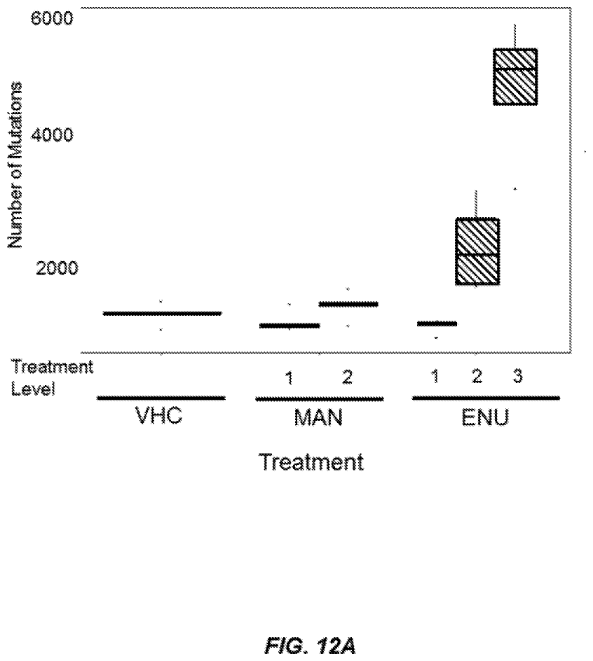

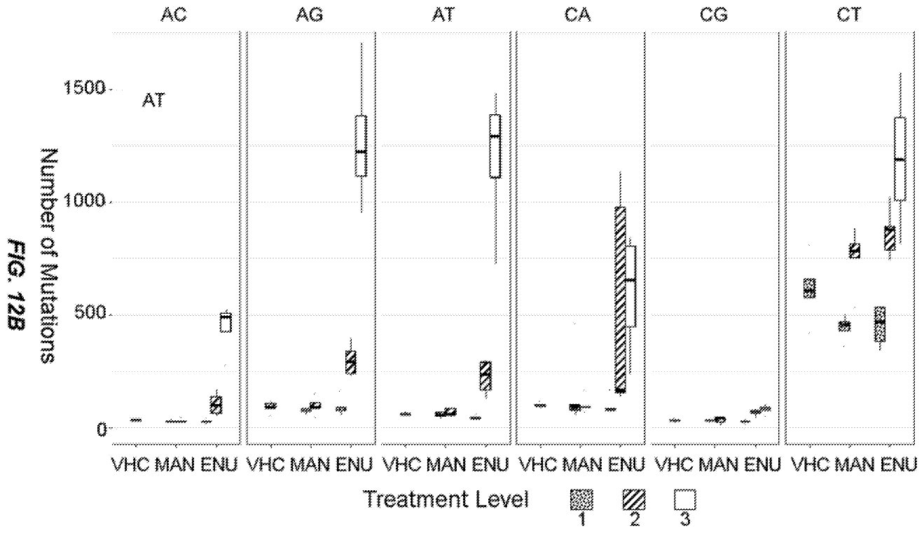

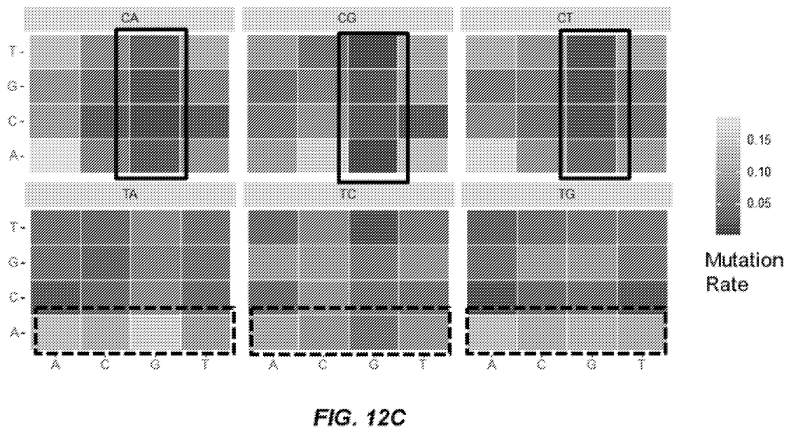

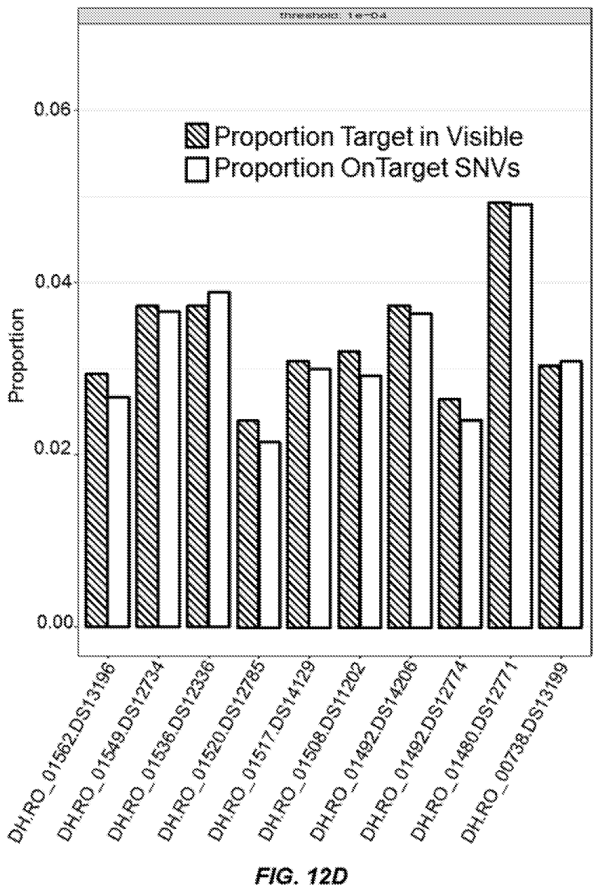

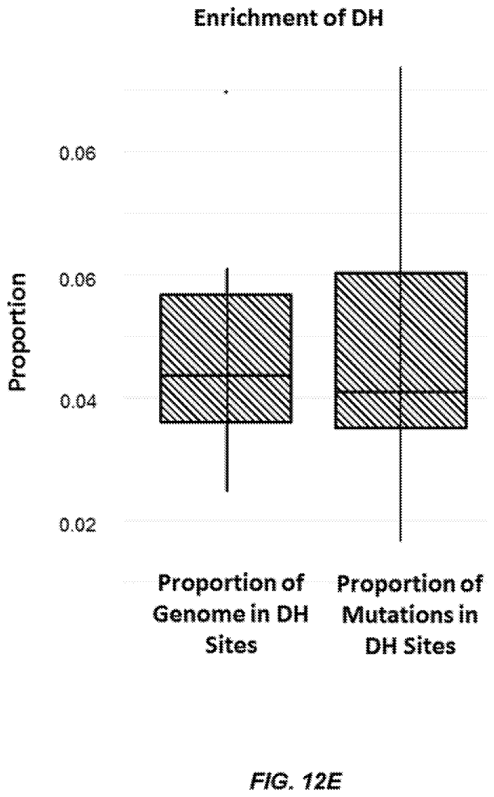

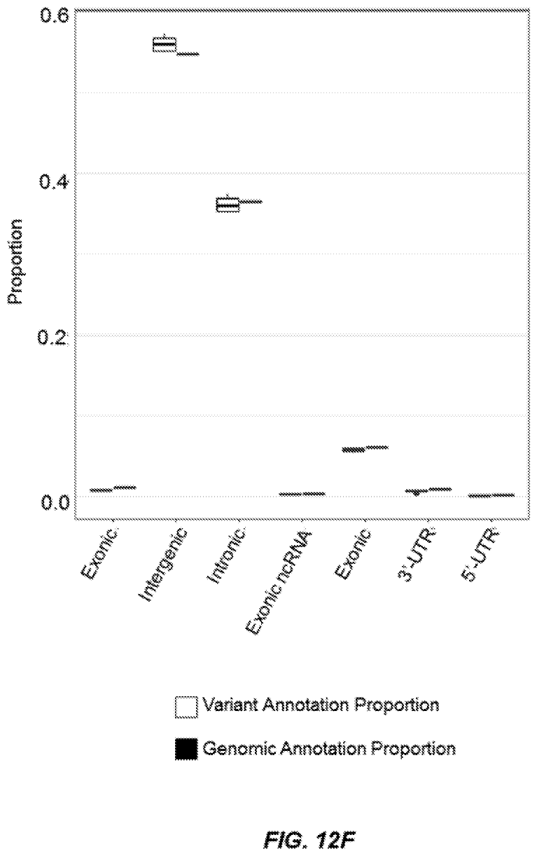

Consistent with these studies, a dose-dependent increase in mutation number of each cell was observed, where a similar number of mutations were detected in the lowest dose of ENU compared to either vehicle control or toxic doses of mannitol (FIG. 12A). Also consistent with previous work in mice using ENU, the most common mutations are T to A (A to T), T to C (A to G), and C to T (G to A). The other three types of base changes were also observed, although C to G (G to C) transversion appears to be rare (FIG. 12B). An examination of the trinucleotide context of the SNVs illustrates two distinct patterns (FIG. 12C). The first pattern is that cytosine mutagenesis appears to be rare when cytosine is followed by guanine. Cytosine that is followed by guanine is commonly methylated at the fifth carbon site in human genomes, which is a marker of heterochromatin. Without being bound by theory, it was hypothesized that 5-methylcytosine does not undergo alkylation by ENU due to inaccessibility in heterochromatin or as a result of unfavorable reaction conditions with 5-methylcytosine compared to cytosine. To test the former hypothesis, locations of the mutation sites were compared to known DNase I hypersensitive sites in CD34+ cells that were catalogued by the Roadmap Epigenomics Project. As seen in FIG. 12D, no enrichment of cytosine variants in DNase I hypersensitivity sites was observed. Further, no enrichment of variants restricted to cytosines was observed in DH sites (FIG. 12E). Additionally, most thymine variants occur where adenine is present before thymine. Genomic feature annotation for the variants was not significantly different from the annotation of those features in the genome (FIG. 12F).

### Example 4

### Massively Parallel Single-Cell DNA Sequencing

Using PTA, a protocol for massively parallel DNA sequencing is established. First, a cell barcode is added to the random primer. Two strategies to minimize any bias in the amplification introduced by the cell barcode is employed: 1) lengthening the size of the random primer and/or 2) creating a primer that loops back on itself to prevent the cell barcode from binding the template (FIG. 10B). Once the optimal primer strategy is established, up to 384 sorted cells are scaled by using, e.g., Mosquito HTS liquid handler, which can pipette even viscous liquids down to a volume of 25 nL with high accuracy. This liquid handler also reduces reagent costs approximately 50-fold by using a 1 μL PTA reaction instead of the standard 50 μL reaction volume.

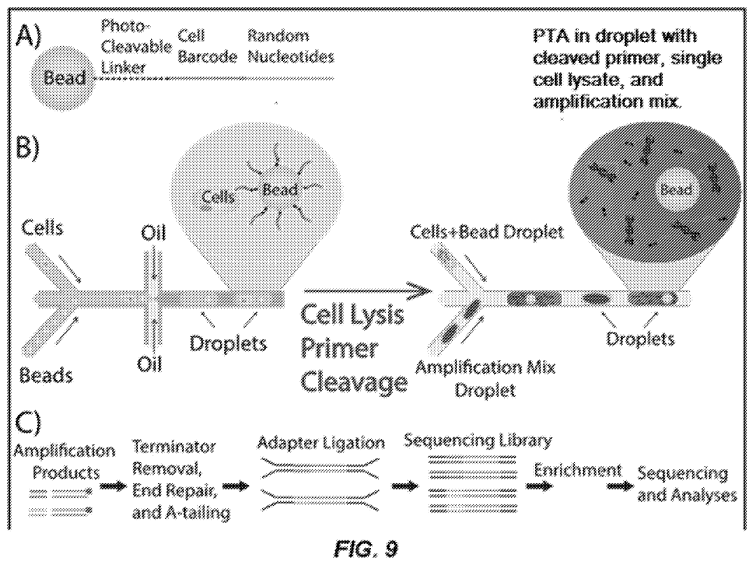

The amplification protocol is transitioned into droplets by delivering a primer with a cell barcode to a droplet. Solid supports, such as beads that have been created using the split-and-pool strategy, are optionally used. Suitable beads are available e.g., from ChemGenes. The oligonucleotide in some instances contains a random primer, cell barcode, unique molecular identifier, and cleavable sequence or spacer to release the oligonucleotide after the bead and cell are encapsulated in the same droplet. During this process, the template, primer, dNTP, alpha-thio-ddNTP, and polymerase concentrations for the low nanoliter volume in the droplets are optimized. Optimization in some instances includes use of larger droplets to increase the reaction volume. As seen in FIG. 9, this process requires two sequential reactions to lyse the cells, followed by WGA. The first droplet, which contains the lysed cell and bead, is combined with a second droplet with the amplification mix. Alternatively or in combination, the cell is encapsulated in a hydrogel bead before lysis and then both beads may be added to an oil droplet. See Lan, F. et al., Nature Biotechnol., 2017, 35:640-646).

Additional methods include use of microwells, which in some instances capture 140,000 single cells in 20-picoliter reaction chambers on a device that is the size of a 3″×2″ microscope slide. Similarly to the droplet-based methods, these wells combine a cell with a bead that contains a cell barcode, allowing massively parallel processing. See Gole et al., Nature Biotechnol., 2013, 31:1126-1132).

### Example 5

### Application of PTA to Pediatric Acute Lymphoblastic Leukemia (ALL)

Single-cell exome sequencing of individual leukemia cells harboring an ETV6-RUNX1 translocation has been performed, measuring approximately 200 coding mutations per cell, only 25 of which have been present in enough cells to be detected with standard bulk sequencing in that patient. The mutation load per cell has then been incorporated with other known features of this type of leukemia, such as the replication-associated mutation rate (1 coding mutation/300 cell divisions), the time from initiation to diagnosis (4.2 years), and the population size at the time of diagnosis (100 billion cells) to create an in silico simulation of the development of the disease. It has been unexpectedly discovered that even in what has been thought to be a genetically simple cancer such as pediatric ALL, there are an estimated 330 million clones with distinct coding mutation profiles at the time of diagnosis in that patient. Interestingly, as seen in FIG. 6B, only the one to five most abundant clones (box C) are being detected with standard bulk sequencing; there are tens of millions of clones that are composed of a small number of cells and are thus less likely to be clinically significant (box A). Accordingly, methods are provided for enhancing the sensitivity of detection so that clones that make up at least 0.01% (1:10,000) of the cells (box B) can be detected, as this is the stratum in which most resistant disease that causes relapse is hypothesized to reside.

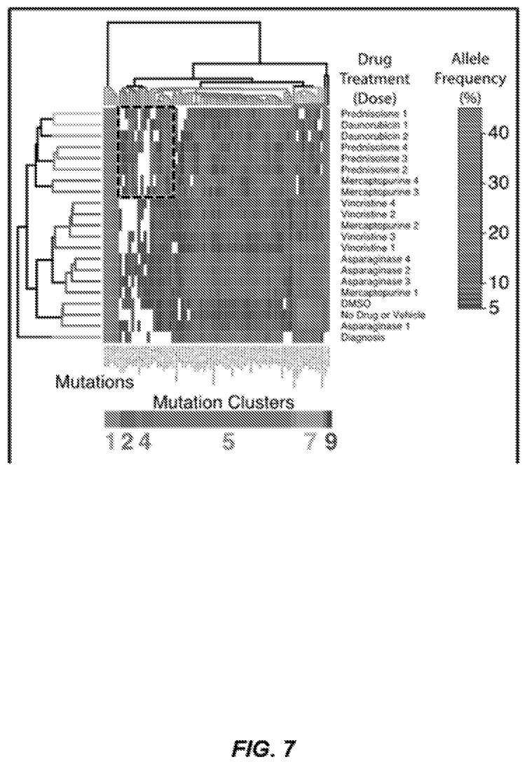

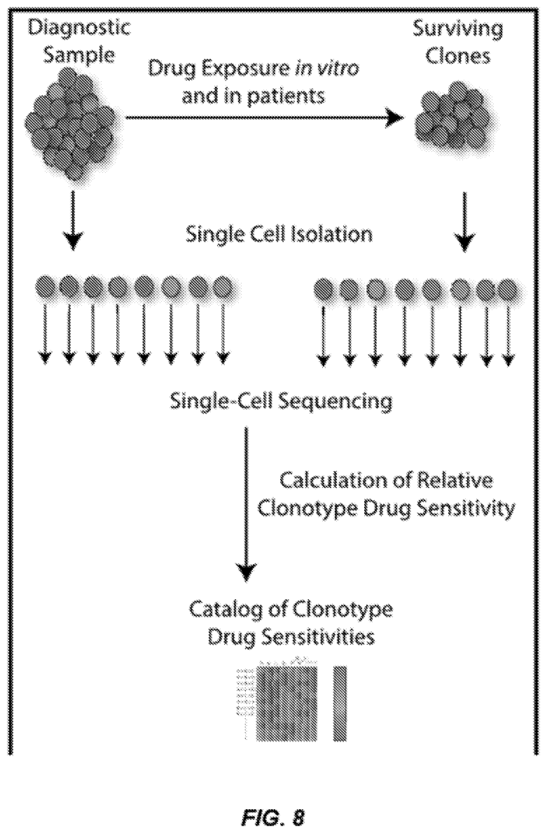

Given such a massive population genetic diversity, it has been hypothesized that there are clones that are more resistant to treatment within a given patient. To test that hypothesis, the sample is placed in culture and the leukemia cells are exposed to increasing concentrations of standard ALL chemotherapy drugs. As seen in FIG. 7, in the control samples and those receiving the lowest dose of asparaginase, the clone harboring an activating KRAS mutation continued to expand. However, that clone proved more sensitive to prednisolone and daunorubicin, whereas other previously undetectable clones could be more clearly detected after treatment with those drugs (FIG. 7, dashed-line box). This approach also employed bulk sequencing of the treated samples. The use of single-cell DNA sequencing in some instances allows a determination of the diversity and clonotypes of the expanding populations.

Creating a Catalog of ALL Clonotype Drug Sensitivities

As shown in FIG. 8, to make a catalog of ALL clonotype drug sensitivities, an aliquot of the diagnostic sample is taken and single-cell sequencing of 10,000 cells is performed to determine the abundance of each clonotype. In parallel, the diagnostic leukemic cells are exposed to standard ALL drugs (vincristine, daunorubicin, mercaptopurine, prednisolone, and asparaginase), as well as to a group of targeted drugs (ibrutinib, dasatanib, and ruxolitinib) in vitro. Live cells are selected and single-cell DNA sequencing on at least 2500 cells per drug exposure will be performed. Finally, bone marrow samples from the same patients after they have completed 6 weeks of treatment are sorted for live residual preleukemia and leukemia, using established protocols for the bulk-sequencing studies. PTA is then used to perform single-cell DNA sequencing of tens of thousands of cells in a scalable, efficient, and cost-effective manner, which achieves the following goals.

From Clonotypes to a Drug Sensitivity Catalog of Drug Sensitivities

Once sequencing data are acquired, the clonotypes of each cell are established. To accomplish this, variants are called and clonotypes are determined. By utilizing PTA, the allelic dropout and coverage bias introduced during currently used WGA methods is limited. A systematic comparison of tools for calling variants from single cells that underwent MDA has been performed, and it was found that the recently developed tool Monovar has the highest sensitivity and precision (Zafar et al., Nature Methods, 2016, 13:505-507). Once the variant calls have been made, it is determined if two cells have the same clonotype, despite some variant calls missing due to allelic dropout. To accomplish this, a mixture model of multivariate Bernoulli distributions may be used (Gawad et al., Proc. Natl. Acad. Sci. USA, 2014, 111(50):17947-52). After establishing that cells have the same clonotype, it is determined which variants to include in the catalog. Genes that meet any of the following criteria are included: 1) they are nonsynonymous variants detected in any of the mutational hotspots or loss-of-function variants (frameshift, nonsense, splicing) that occur in a known tumor-suppressor gene identified in the large pediatric cancer genome sequencing projects; 2) they are variants that are recurrently detected in relapsed cancer samples; and 3) they are recurrent variants that undergo positive selection in the current bulk-sequencing studies of residual disease as ALL patients undergo 6 weeks of treatment. If clones do not have at least two variants meeting these criteria, they are not included in the catalog. As more genes associated with treatment resistance or disease recurrence are identified, clones may be “rescued” and included in the catalog. To determine whether a clonotype underwent positive or negative selection between control and drug treatment, Fisher's exact test is used to identify clones that are significantly different from the control. Clones will only be added to the catalog when at least two concordant combinations of mutations are shown to have the same correlation with exposure to a specific drug. Known activating mutations in oncogenes or loss-of-function mutations in tumor suppressors in the same gene will be considered equivalent between clones. If clonotypes are not exactly concordant, the mutations in common will be entered into the catalog. For example, if clonotype 1 is A+B+C and clonotype 2 is B+C+D, the B+C clonotype will be entered into the catalog. If genes that are recurrently mutated in resistant cells with a limited number of co-occurring mutations are identified, those clones may be collapsed into functionally equivalent clonotypes.

### Example 6

### Measuring Rates and Locations of CRISPR Off-Target Activity in Single Human Cells

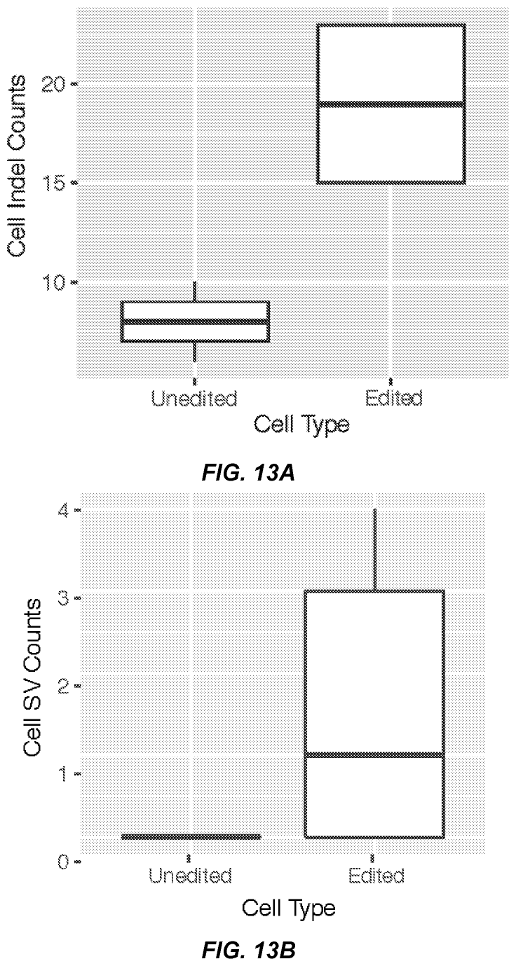

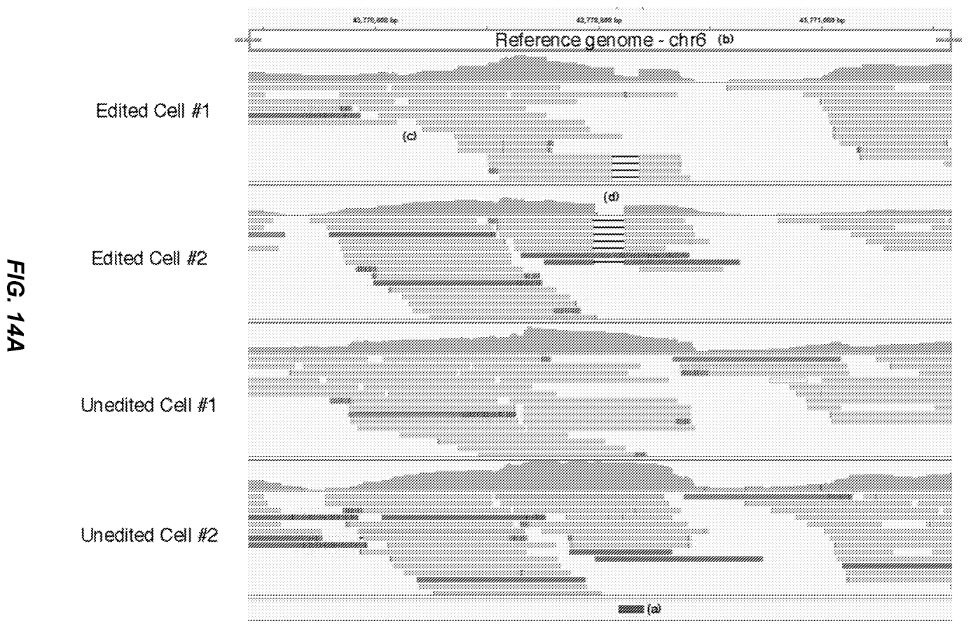

Taking advantage of the improved variant calling sensitivity and precision of PTA in single cells, quantitative measurements of CRISPR-mediated genome editing with specific guide RNAs with high sensitivity in single cells was conducted. Single cells were subjected to the general PTA methods of Example 4. Cell indel and SV counts were compared for both unedited and edited cells (FIG. 13A and FIG. 13B).

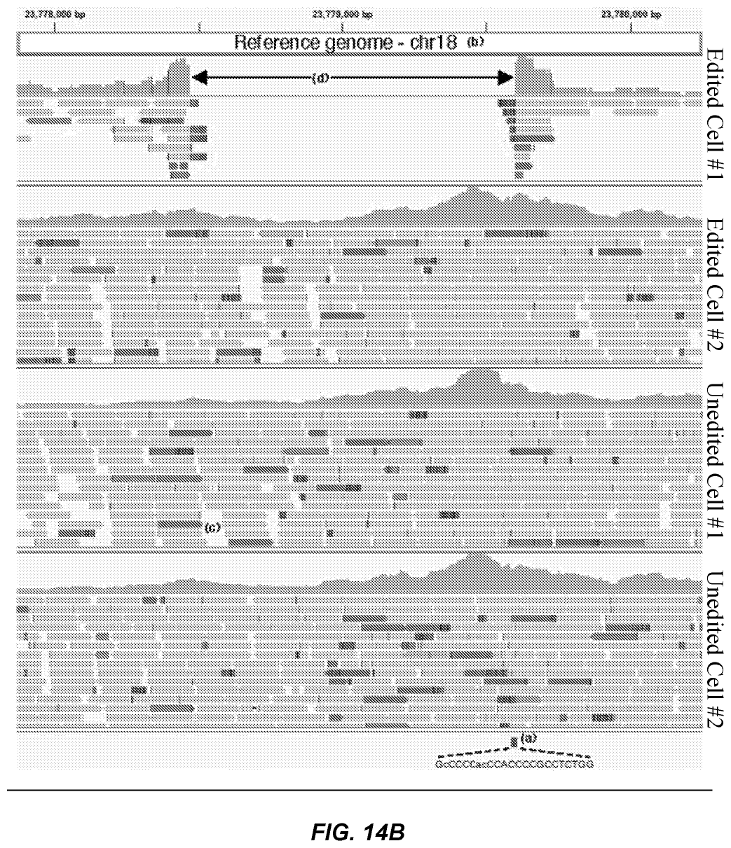

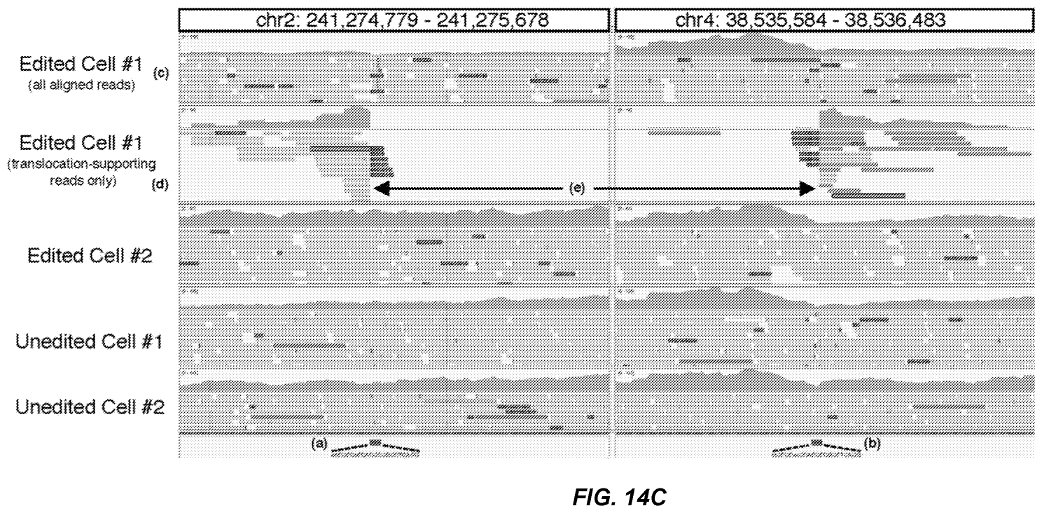

Types of structural variation these genome editing methods can induce in single human cells was also examined, and the results shown in FIGS. 14A-14C. As shown in FIG. 14A, The target region is denoted at bottom (a) and is found on chromosome 6, between positions 43,770,818 and 43,770,841 (b). Sequence data in the form of paired end reads (small horizontal bars without dashes) indicate concordance between the single cell sequence data and the target genome (c). Dashes within reads indicate genomic deletions relative to the reference genome (d). In this example, both edited cells show a deletion (d) overlapping the target site (a). In contrast, the two unedited cells contain reads that indicate they are concordant with the reference genome at this location and thus no editing has occurred. FIG. 14B shows detection of a large (>1 KB) deletion resulting from CRISPR-induced editing that is restricted to edited cell #1. The target region is denoted at bottom (a) and is found on chromosome 18 between positions 23,779,588 and 23,779,611 (b). Sequence data in the form reads (small colored horizontal bars, typically grey) indicate concordance between the single cell sequence data and the target genome (c). Regions with an abrupt drop in aligned reads indicate a deviation from the reference genome at these locations. In this case, the sudden loss of read coverage between positions 23,778,472 and 23,779,607 on chromosome 18 indicate a large deletion in edited cell #1 (d). This deletion is identified as a CRISPR-induced deletion because the right-most breakpoint in the figure overlaps with a region of the genome that is highly similar to the target site (a) and the deletion is not present in the unedited cells. Lowercase letters in (a) indicate bases that differ from the target site. FIG. 14C shows detection of an inter-chromosomal translocation between chromosome 2, position 241,275,213 and chromosome 4, position 38,536,006 in edited cell #1. The translocation breakpoints overlap with gRNA off target regions in each chromosome that are similar to the gRNA target site and are denoted at bottom [(a) and (b)]. The left panel represents reads aligned to the chromosome 2 region containing the breakpoint and the right panel represents reads aligned to the chromosome 4 region containing the breakpoint. Edited cell #1 is split into two views: a view with all reads aligning to the regions surrounding the breakpoint (c), and a view of the same region but only showing read pairs that are evidence for the translocation (d). For read pairs that support the translocation, one read of a pair aligns to chromosome 2 with an abrupt drop in coverage at the breakpoint, and the other read aligns to chromosome 4, also with an abrupt drop in read coverage at the breakpoint (e). This translocation is identified as a CRISPR-induced translocation because at least one of the translocation breakpoints overlaps with a region of the genome that is highly similar to the target site in the edited cells (in this case two: a and b) and there is no evidence of the translocation in the unedited cells. Lowercase letters in (a) and (b) indicate bases that differ from the target site.

To confirm putative off target sites, as well as to assess the precision of variants calls with increasing number of guide RNA genome mismatches, microfluidic high-throughput PCR-based resequencing of putative off-target sites in all cells was also performed (data not shown).

### Example 7

### Estimation of Age

Data is collected for a population of at least 1000 human subjects, including geographic location (most time spent in), sex, age, ethnicity, and genomic mutation frequencies and locations established using the PTA method. Samples are run in duplicate, and are obtained from one or more tissues from each of the subjects. Standard curves are generated correlating variables such as geographic location (area most time lived in), sex, age, ethnicity, mutation frequencies, mutation locations, or other data obtained vs. the age of the subject. A genome from a sample of a subject of unknown age is sequenced using the PTA method, and standard curves are used to determine the age of the individual. If additional information is known about the subject (ethnicity, geographic location), this is used to further improve the prediction.

### Example 8

### Identification and Diagnosis of Clinical Bacteria Samples

A sample of cells from a subject with a suspected bacterial infection is obtained, and subjected to single cell genomic sequencing using the PTA method. Mutations identified with the PTA method are compared to mutations conferring known antibiotic resistance, or used to identify the strain of bacteria. This information is used to select appropriate methods of treatment, such as an effective antibiotic.

### Example 9

### Identification of Microbial Species and Genes

Samples of water are collected from various sources, such as deep sea vents, ocean, mines, streams, lakes, meteorites, glaciers, or volcanoes. Samples are subjected to a 20 micro pre-filter to remove particulates, then fractionated into size groups such as 3-20 micron, 0.8-3 micron, 0.1-0.8 micron, and 50 kDa to 0.1 micron. Samples are then processed to isolate individual cells or optionally processed in bulk. Genomic, plasmid, or other DNA is isolated using standard techniques, subjected to the PTA method, and then sequenced. After reassembly of genome sequences, known species are identified and unknown species and/or genes are characterized for potential industrial applications.

### Example 10

### Measuring Unintended Insertion Rates of Gene Therapy Approaches

Taking advantage of the improved variant calling sensitivity and precision of PTA in single cells, quantitative measurements of unintended insertion rates of gene therapy approaches with high sensitivity in single cells is conducted. The method can detect the insertion of specific sequences in a non-desired location by detecting the surrounding sequence to determine if the gene therapy approach is causes insertion or modification of the host genome. Nucleic acids encoding for a gene which produces a protein are introduced into a viral carrier vector, and then delivered to one or more cells in an organism or in-vitro. The virus delivers the nucleic acids to the nucleus, and the nucleic acid is transcribed into mRNA. After translation of the mRNA, the protein is produced. Cells modified by this gene therapy method are sequenced using the general PTA method described in Example 4, and mutations (mutation frequency and location/pattern) caused by the gene therapy method are detected.

### Example 11

### Calling CNV with PTA in Primary Cancer Cells

Primary leukemia cells were used to perform further validation studies of a PTA protocol for SNV and copy number variation (CNV) calling compared to MDA, as well as to recently developed or improved commercially available kits, following the general methods of Example 1, the PTA protocol showed further increases in coverage breadth and continued to be the most uniform method based on CV calculations at base pair resolution (FIG. 19). PTA also remained the most sensitive method for SNV calling at all sequencing depths, and now had the highest SNV calling specificity by changing to a low temperature lysis. Methods that rely on PCR (WGA Kit 3, PicoPlex Gold) also continued to show decreased specificity at increasing sequencing depths, although the drop in specificity was significantly improved over MALBAC and the previous version of PicoPlex.

To estimate the accuracy of calling CNV of different sizes for each method, each bam file was sampled to 300 million reads and the CV measured at increasing bin sizes (FIG. 5J). PTA was found to have the lowest CV compared to all other WGA methods at every bin (FIG. 5J). WGA kit 2 and PicoPlex Gold had sharp declines in CV values at increasing depths. This particular leukemia sample had known CNV on 5q and 11q. As expected, the bulk sample and single cells all had a single copy of the X chromosome detected. CNV analysis found the 5q deletion to be clonal while the 11q change was only found in a subset of cells (FIG. 5K, shaded arrows). Bulk data suggested there may be a deletion on 12p, but it was not called in the bulk sample. Two of the five single cells were found to have CNV at the same location, suggesting single-cell CNV profiling may be more sensitive, as well as a better strategy for estimating the percent of cells in a tissue that have a given copy number change.

### Example 12

### Measurement of SNV Rates in Kindred Cells

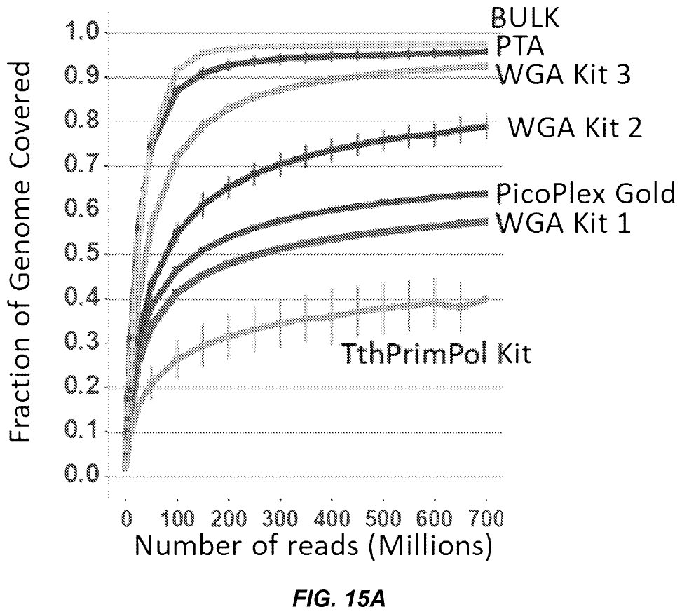

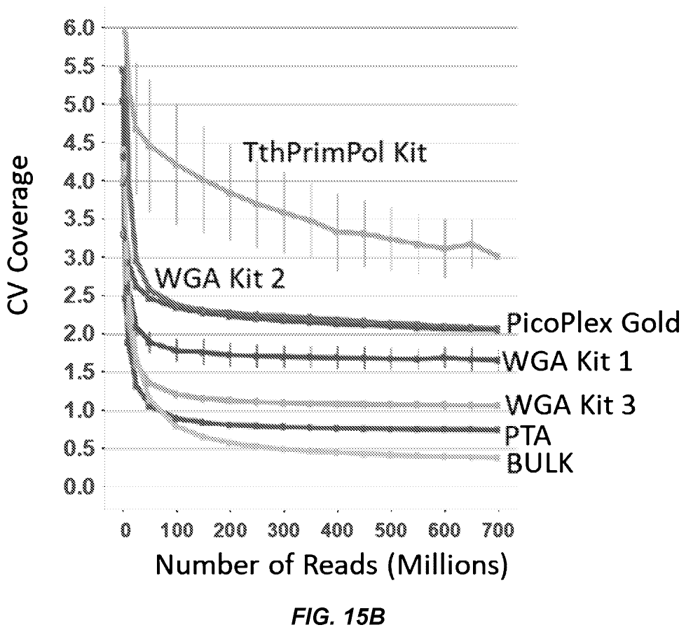

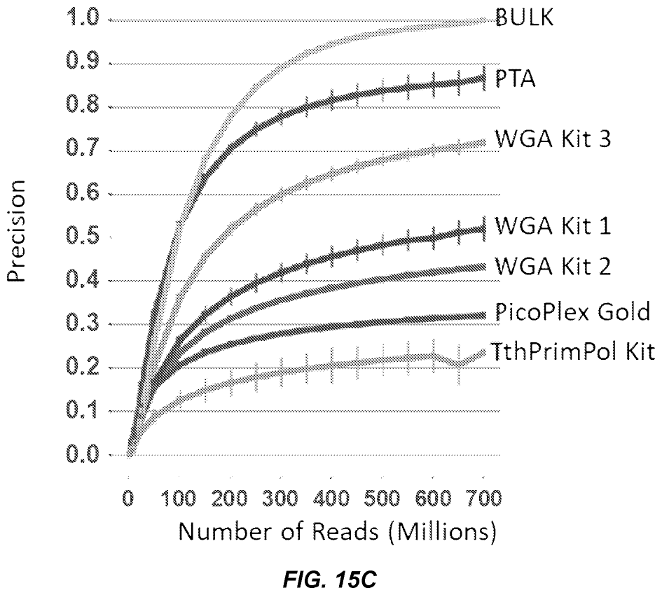

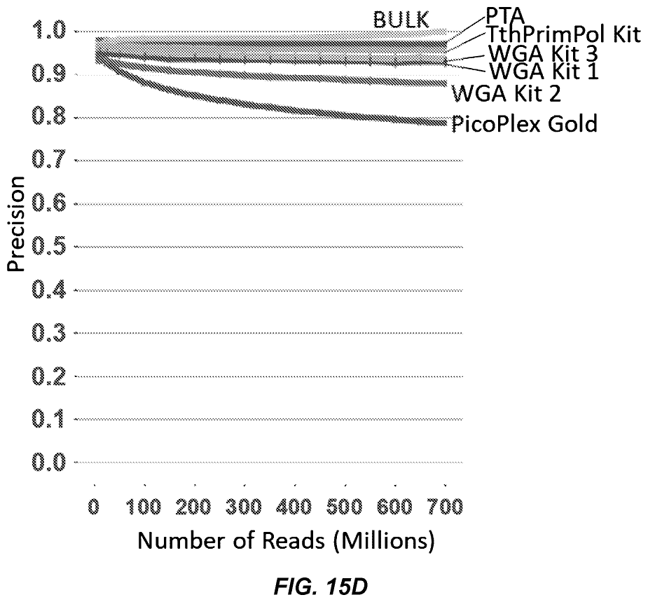

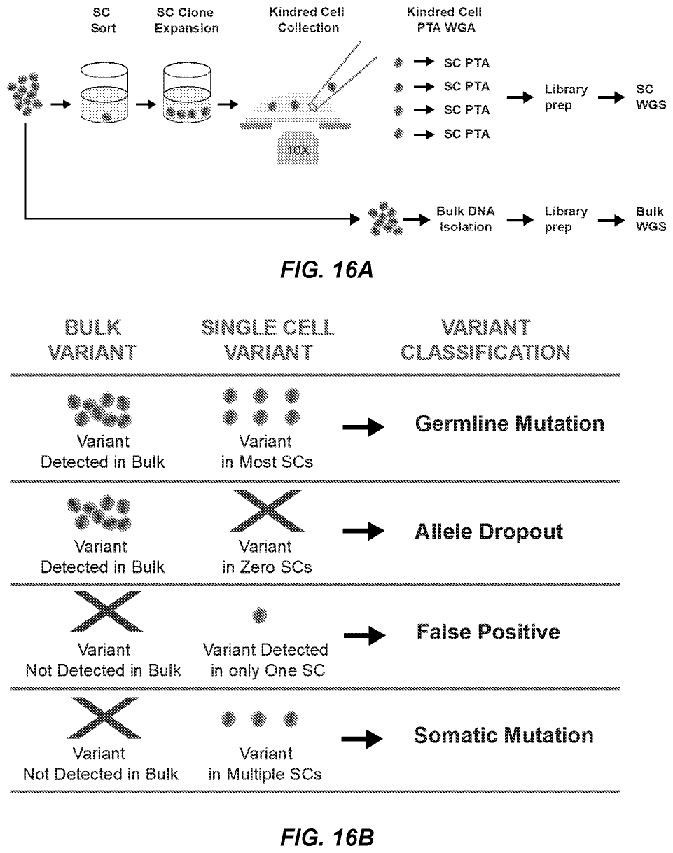

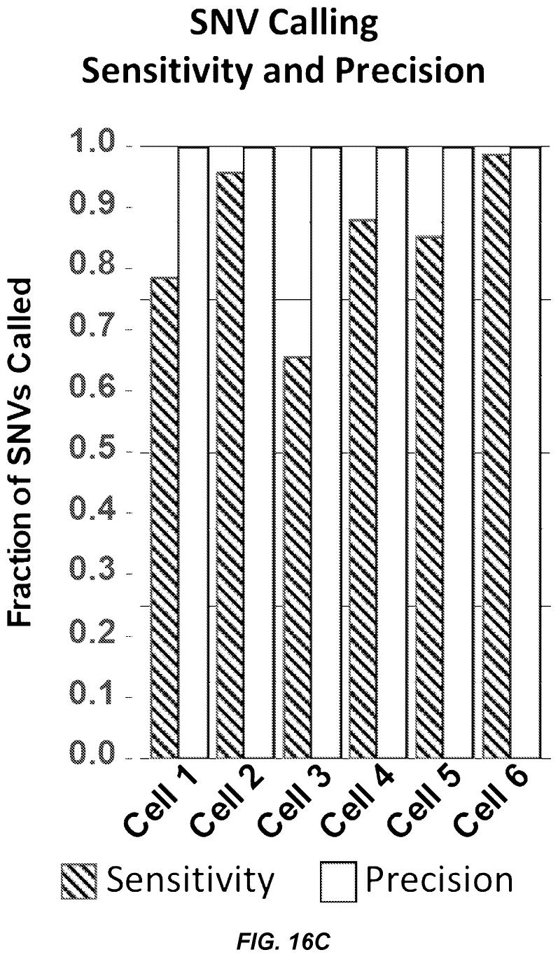

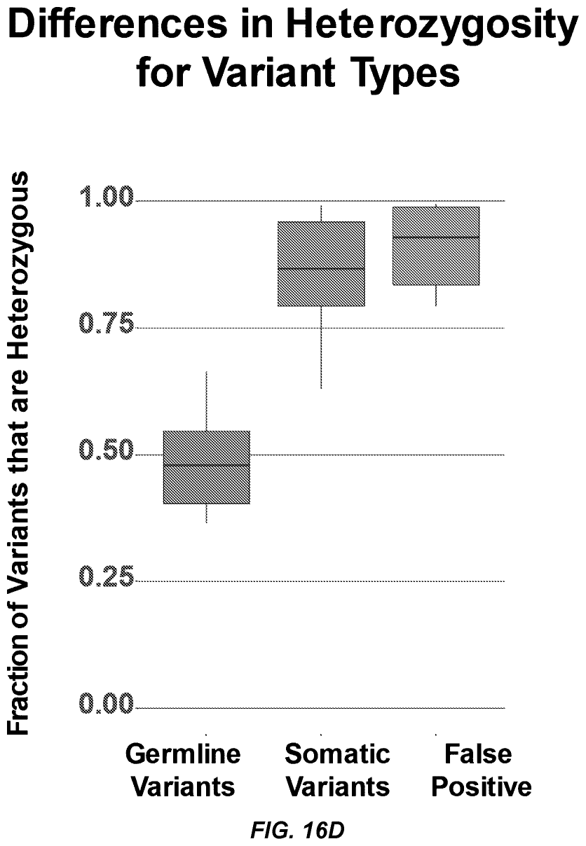

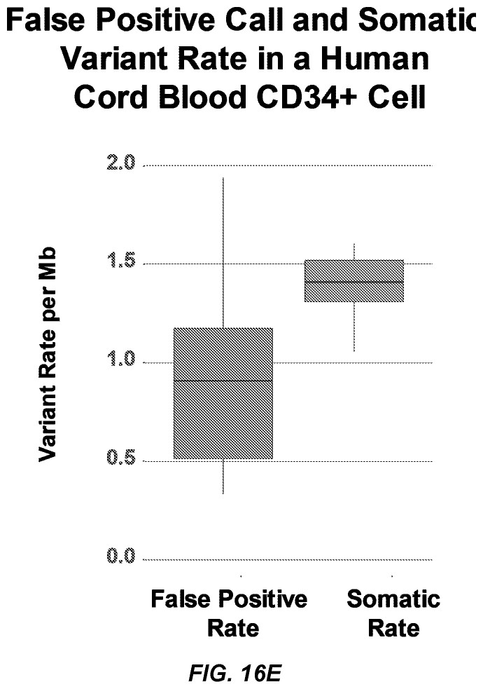

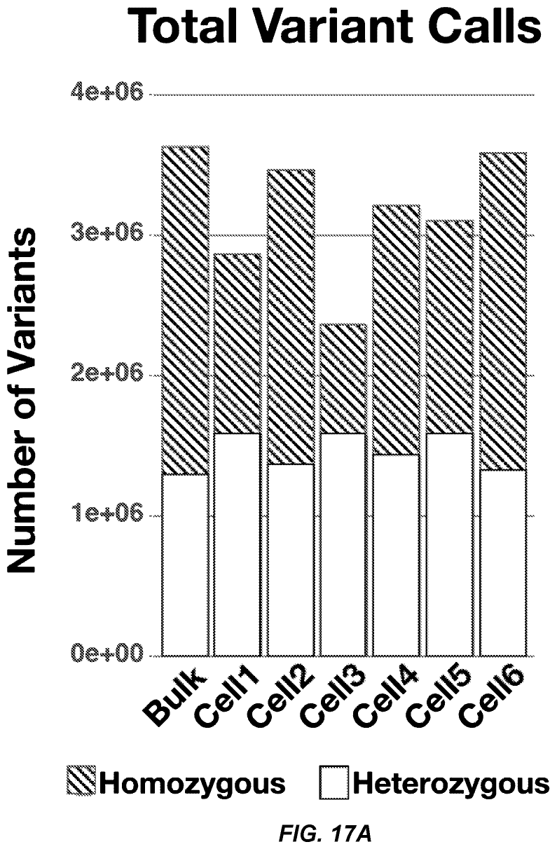

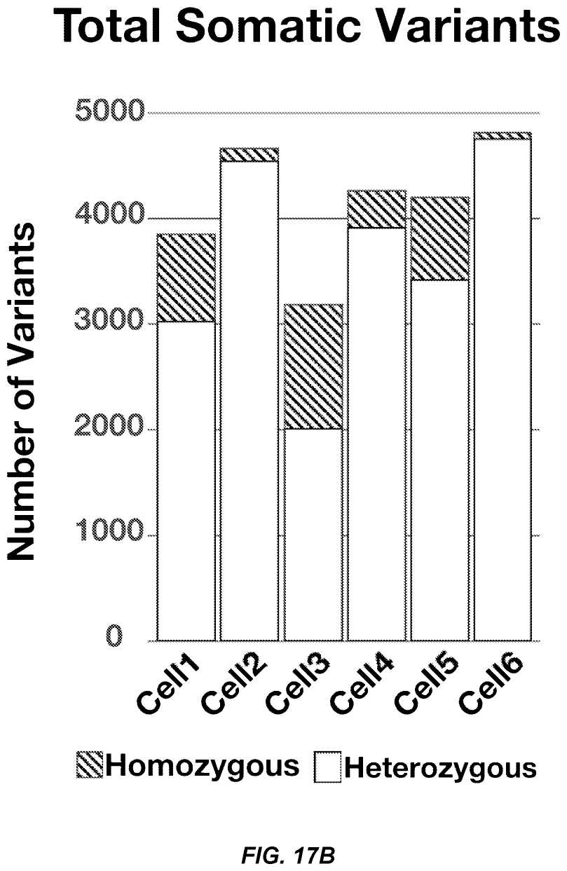

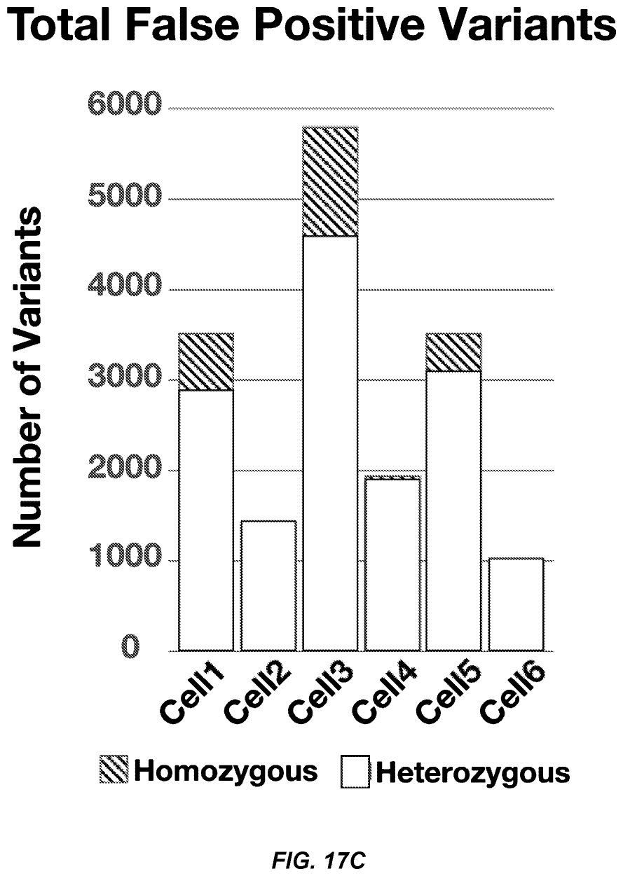

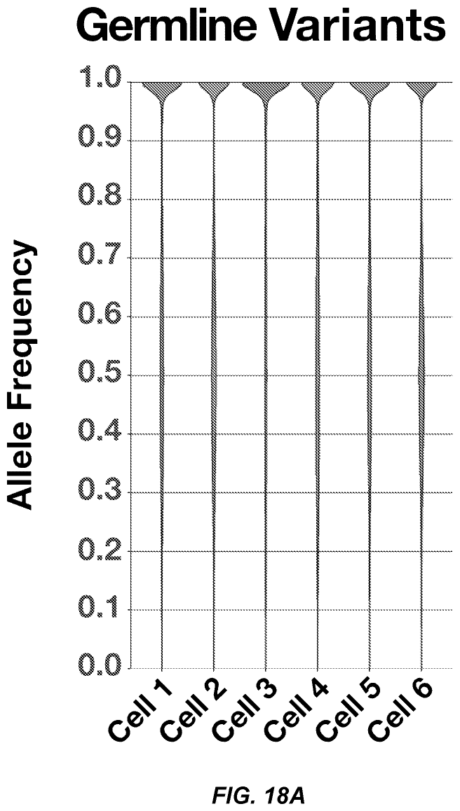

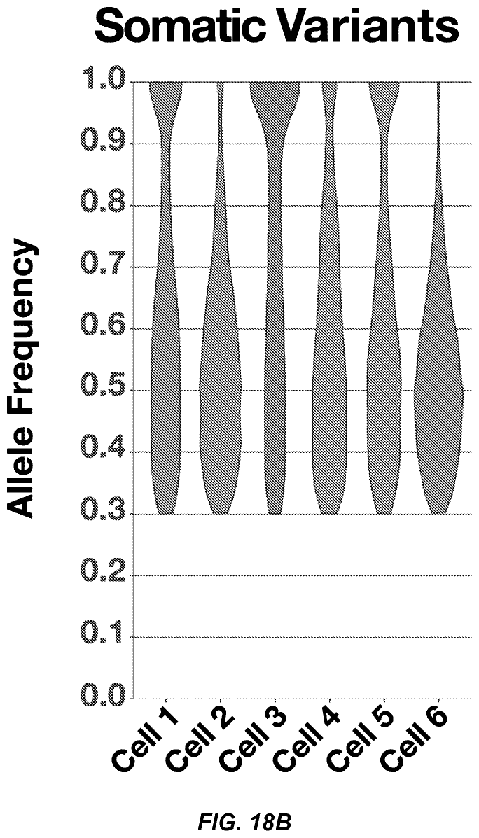

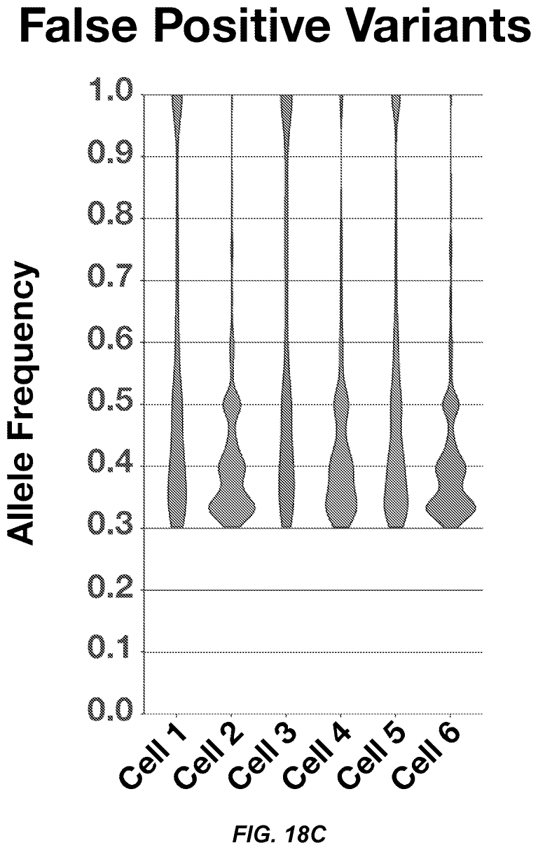

Kindred cell studies were performed by plating single CD34+ CB cells into a single well, followed by expansion for five days (FIG. 16A). Single cells were then reisolated from that culture to compare variant calling of cells that are almost genetically identical. Further, using the bulk as a reference, germline, false positive, and somatic variant calls were discriminated (FIG. 16B). With this approach, and again using the bulk sample as the ground truth, variant calling precision was determined with the low temperature protocol using GATK4 joint genotyping increased to 99.9% (FIG. 16C). Further, most of these primary cells had similar or improved variant detection sensitivity. However, there was one cell that had significantly lower variant calling sensitivity, which, without being bound by theory could be the result of manually manipulating fragile primary cells. In addition, two cells with higher variant calling sensitivity had fewer homozygous somatic variant calls, which may be the result of decreased allele dropout (FIG. 15B). The false positive variants in those had a skewing to lower allele frequencies, which without being bound by theory, could be explained by these rapidly dividing cells being tetraploid in late S or G2/M phase of the cell cycle with only one of four alleles acquiring a copying error (FIGS. 17A-17C). Homozygous false positive calls were observed to cluster at specific locations while the heterozygous calls did not. Without being bound by theory, this could be the result of loss or lack of template denaturating at one allele at those locations during the amplification, which does not appear to be dependent on the GC content of the genomic region (FIGS. 18A-18C). Most false positive and somatic variants were called heterozygous, which is consistent with the model that only one allele is mutated as a result of copying errors or during development, respectively (FIG. 16D). False positive and somatic mutation rates were measured in neonatal CD34+ hematopoietic cells, which were estimated to be 0.9 and 1.4 per Mb of genome, respectively.

### Example 13

### Measuring Rates and Locations of CRISPR Off-Target Activity in Single Human Cells

The continued development of genome editing tools shows great promise for improving human health, from correcting genes that result in or contribute to the formation of disease to the eradication of infectious diseases that are currently incurable. However, the safety of these interventions remain unclear as a result of an incomplete understanding of how these tools interact with and permanently alter other locations in the genomes of the edited cells. Methods have been developed to estimate the off-target rates of genome editing strategies, but all of the tools that have been developed to date interrogate groups of cells together, limiting the capacity to measure the per cell off-target rates and variance between cells, as well as to detect rare editing events that occur in a small number of cells. Single cell cloning of edited cells has been performed, but could select against cells that acquire lethal off-target editing events and is impractical for many types of primary cells.

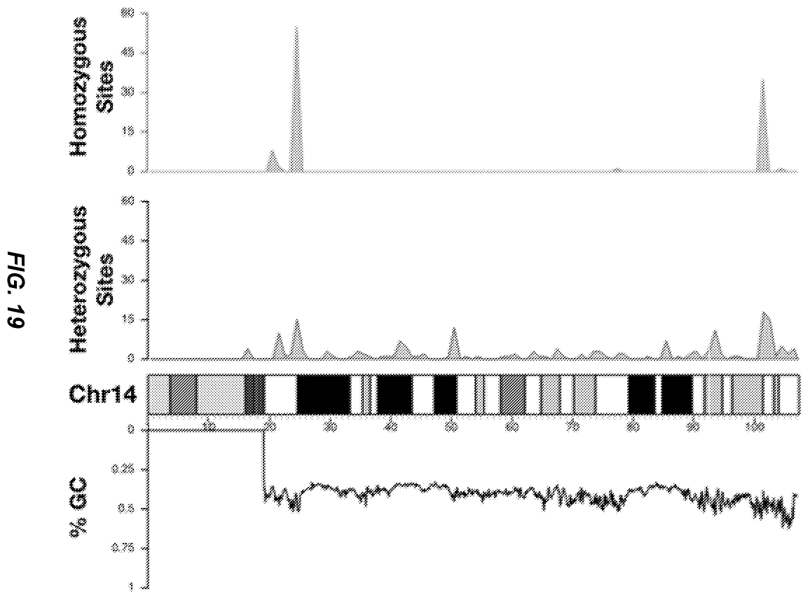

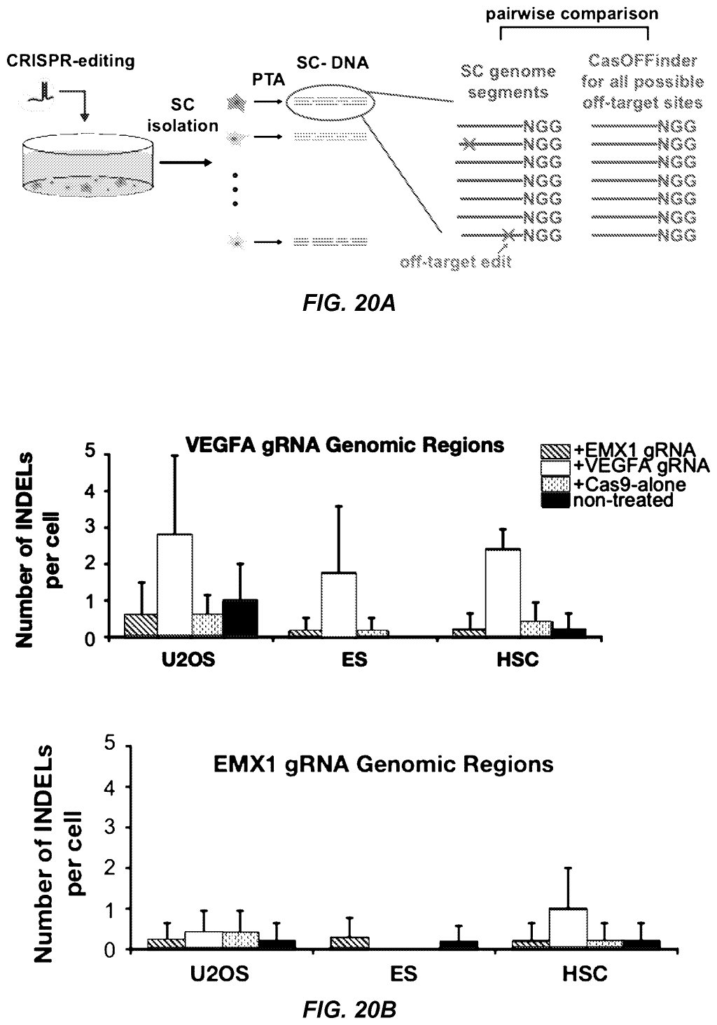

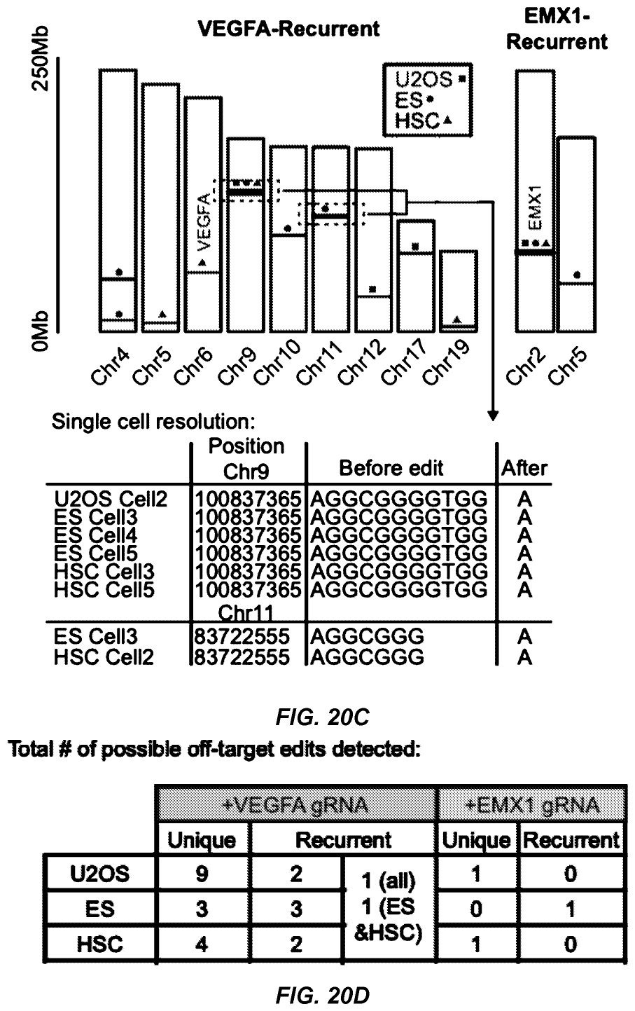

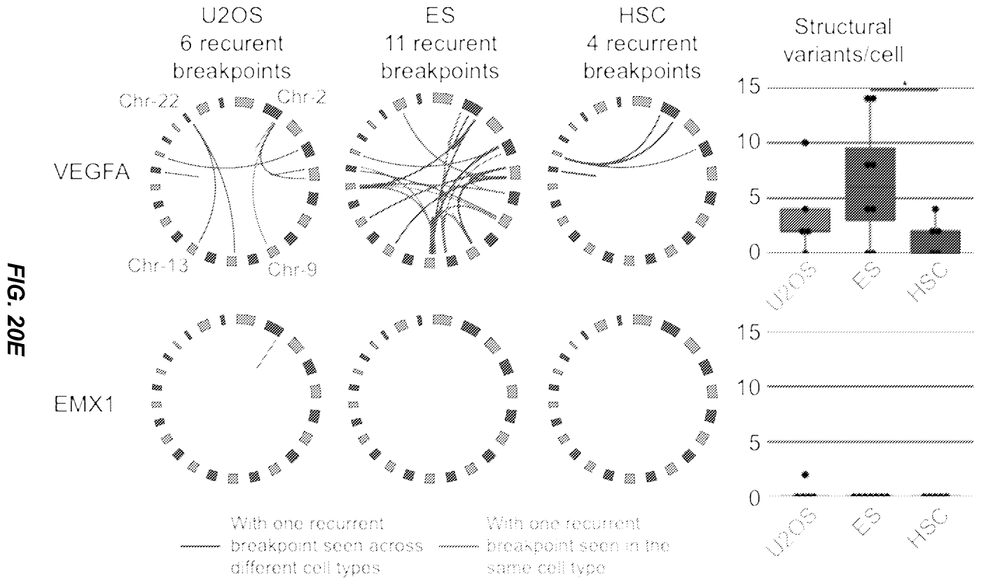

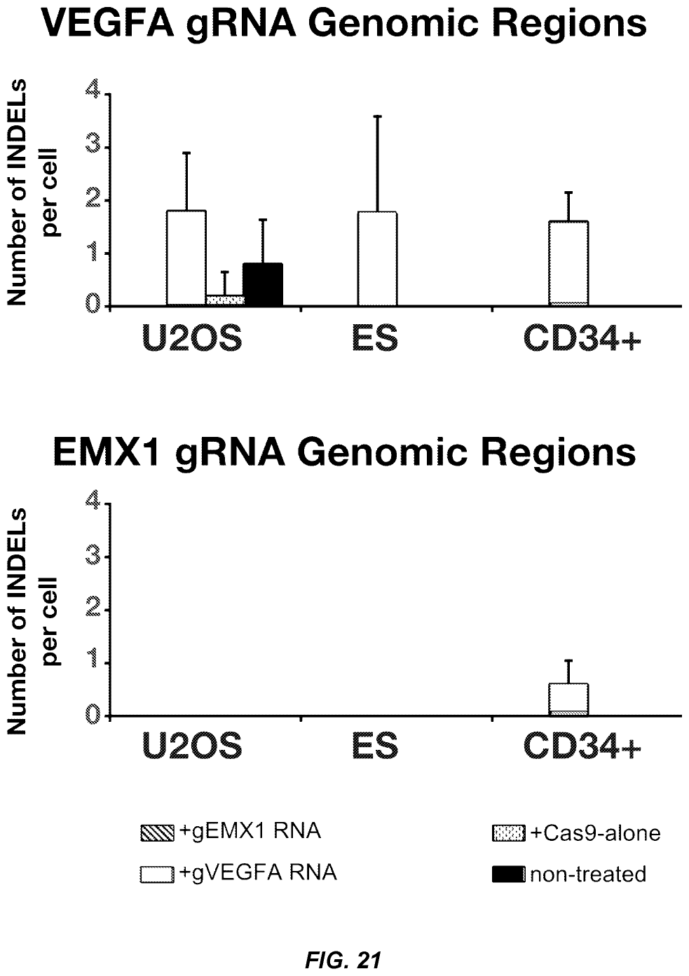

Taking advantage of the improved variant calling sensitivity and specificity of PTA, quantitative measurements of CRISPR-mediated genome editing with specific guide RNAs (gRNA) in single cells were obtained (FIG. 20A). Three cell types were utilized for these studies: U20S osteosarcoma cell line, primary hematopoietic CD34+ CB cells, and embryonic stem (ES) cells. In addition, two previously described gRNAs were employed, one that is known to be precise (EMX1), and one that is known to have high levels of off-target activity (VEGFA). To identify indels with high specificity, variant calling was restricted to genome locations that had a perfect match to the PAM sequence and up to five mismatches to the protospacer (FIG. 16A).

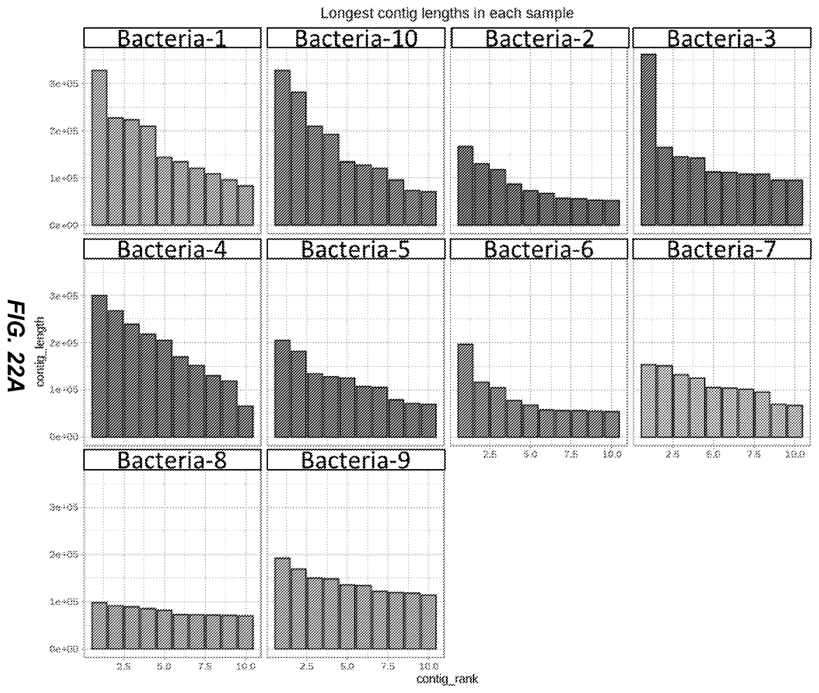

Compared to control cells that either received Cas9 alone or had a mock transfection, there were more off-target indels in the VEGFA edited cells that showed wide cell-to-cell variance while only a small number of off-target EMX1 editing events were detected (FIG. 20B). It was noted that most of the presumed false positive edits that were seen in the control cells were single base pair insertions. Removal of non-recurrent single base pair insertions further improved the specificity of indel calling (FIG. 21). Most, but not all of recurrent off-target sites were cell-type-specific, further supporting the finding that the general chromatin structure of a cell type influences off-target genomic locations (FIG. 20D). Structural variant (SV) calling was performed to identify genome editing-induced SV where it was required regions around both breakpoints have a perfect match to the PAM sequence and allowed up to 5 mismatches with the protospacer. Increased numbers of SV with the VEGFA guide RNA were measured, with only one SV detected in EMX1-edited cells and no SV detected in control cells (FIG. 20E). Recurrent VEGFA-mediated SV was detected, some of which were cell-type specific, and greater SV was detected in the ES cells (FIG. 20C).

### Example 14

### Bacterial Genome Assembly with PTA

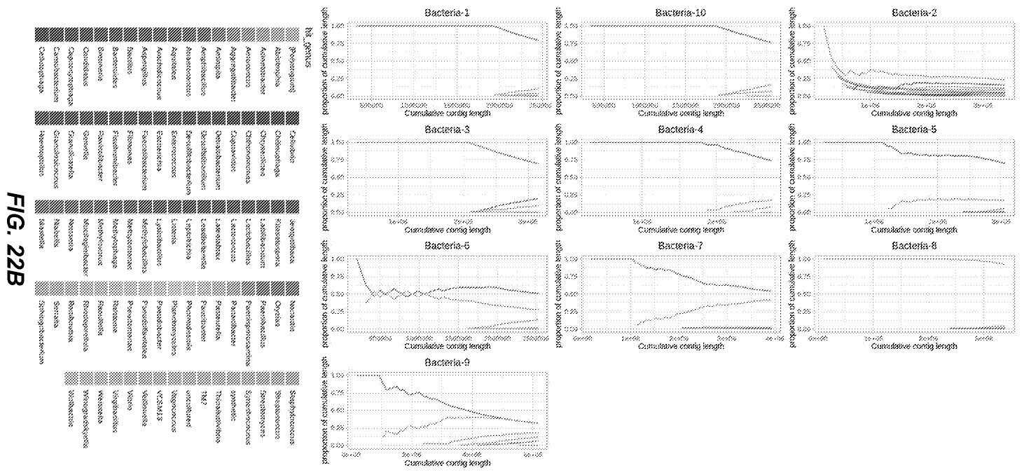

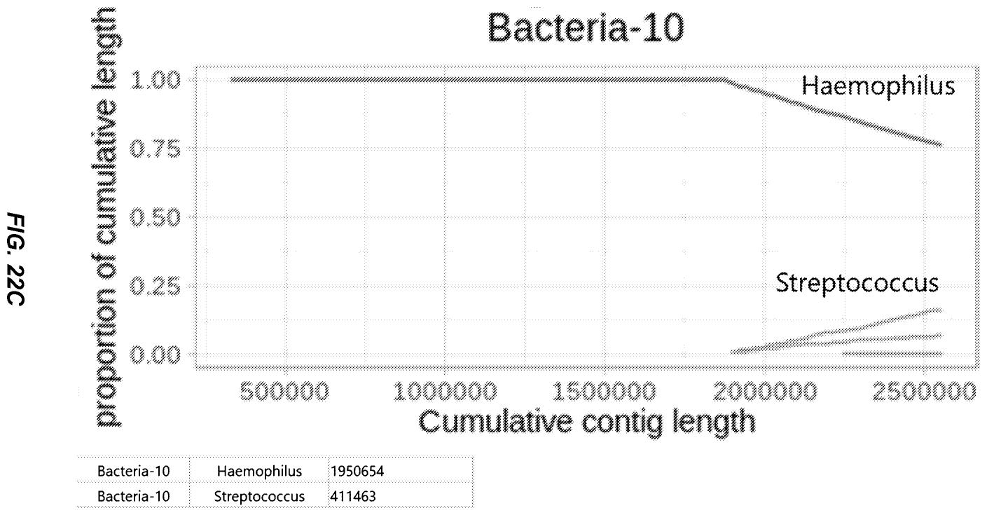

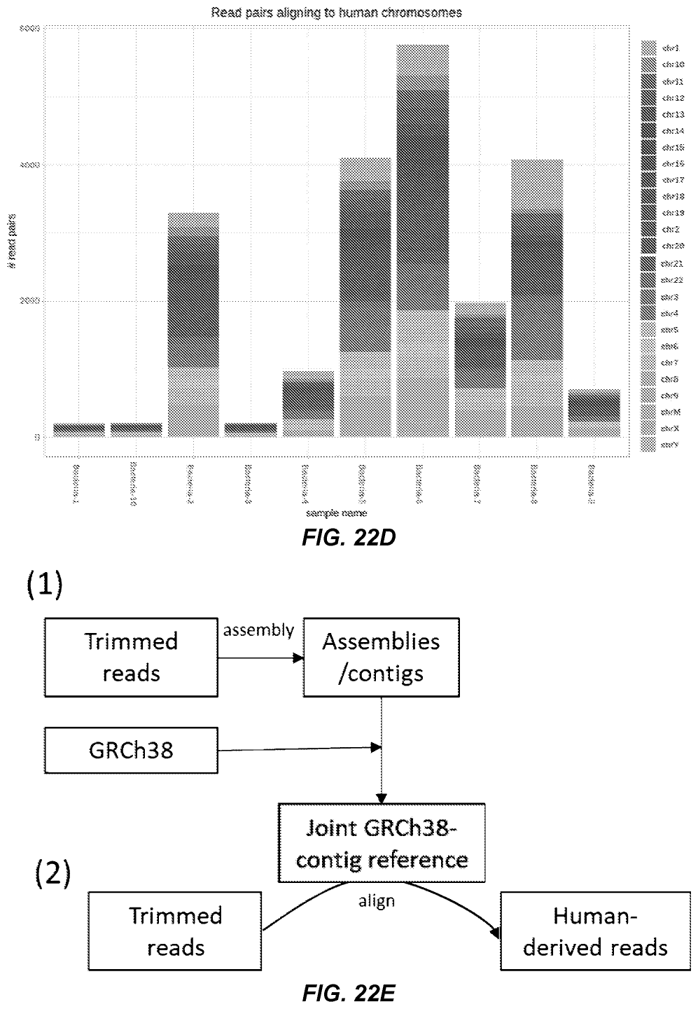

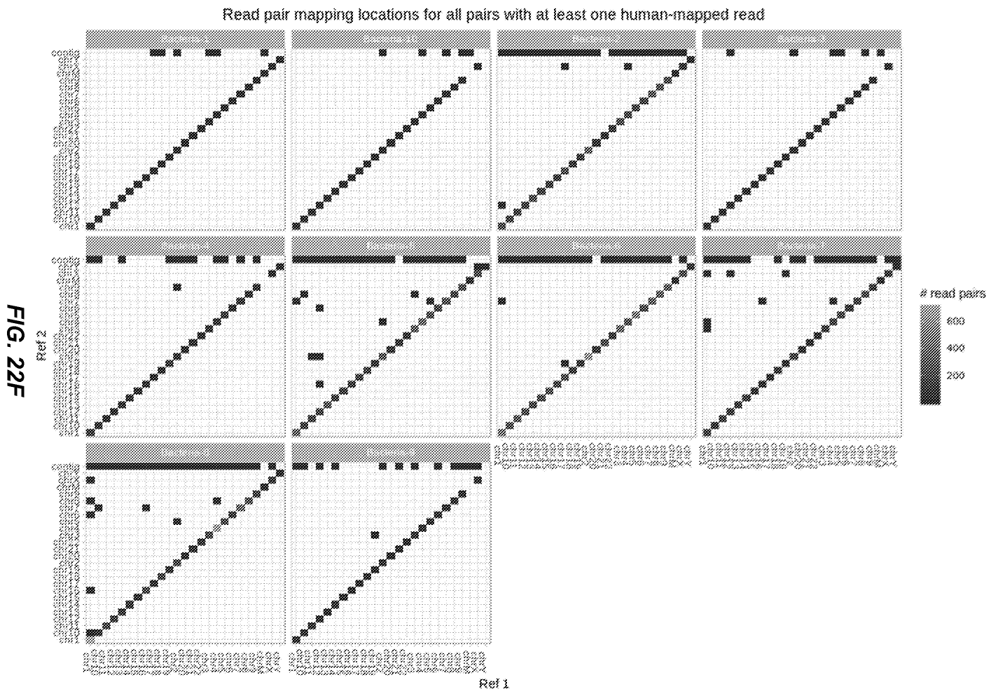

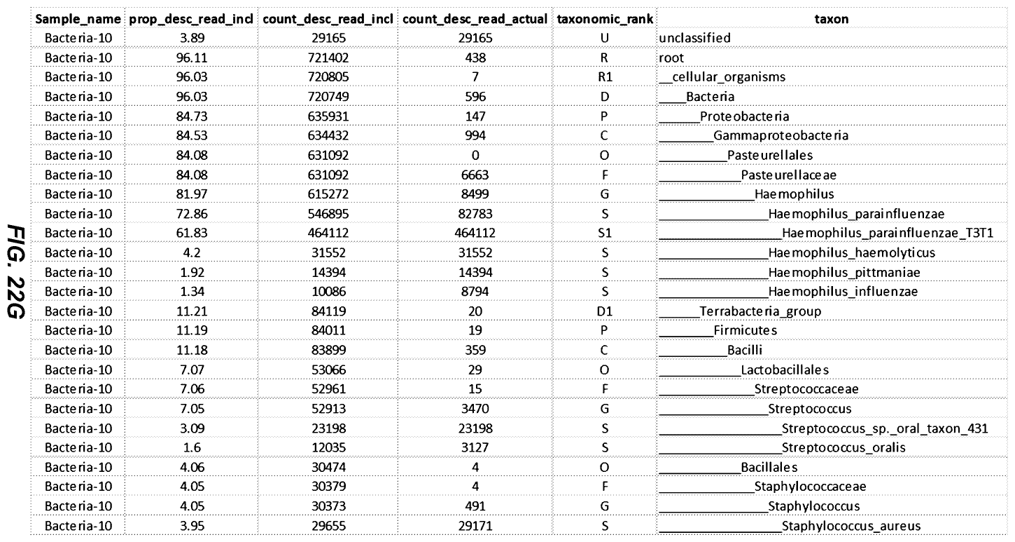

Buccal swabs were obtained and cultured overnight in LB media. Single colonies of bacteria were sorted into a 96 well plate as individual samples, and the general PTA method of Example 1 was conducted on each well to prepare each sample for sequencing. 1-2 million reads were obtained per sample, and reads were assembled using SPAdes (contig-based approach). Data for longest contigs of 10 different bacteria samples are shown in FIG. 22A. For in-silico analysis of sequencing data, each sample's contigs were sequentially added in order of decreasing length (FIG. 22B). Data for bacterial sample 10 is shown in FIG. 22C. Then, the proportion of total assembly assigned to each genus was determined. Contaminant sequences are present with small fragments of genomic DNA; these are identifiable as being the smaller contigs in a dataset (>5 KB, FIG. 22D). Read pairs were considered human-derived if both reads aligned to GRCh38 in joint GRCh38-contig reference (FIGS. 22E-22F). Alternatively, an assembly-free approach was used for all the samples (e.g., Kraken) by assigning reads to taxa using k-mers from reference databases. Results from a read-based approach for bacteria sample 10 is shown in FIG. 22G, and was consistent with the contig-based approach.

### Example 15

### Preimplantation Genetic Testing with PTA

Non-invasive preimplantation genetic screening (NIPGS) is performed by preparing 20 cultured embryos (frozen or fresh) according to the general methods of Kuznyetsov et al. (2018) PLoS ONE, 13(5): e0197262. Briefly, each embryo is transferred on day 4 of culture to fresh Global HP medium with HSA and is cultured under oil until reaching the blastocyst stage (on day 5 or 6). Upon reaching a fully expanded blastocyst, each blastocyst undergoes laser assisted trophectoderm biopsy, followed by laser collapse, which allows the BF to mix with the BCCM. The embryo is then transferred to cryopreservation medium and frozen by vitrification. After removal of the embryo, the combined BCCM and BF samples are collected and frozen at −80 C until tested. After extraction of nucleic acids from BCCM/BF samples, the nucleic acids are subjected to the general PTA methods of Example 1. The resulting genomic DNA libraries generated from PTA are then analyzed for genetic mutations, such as chromosomal abnormalities.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of determining a mutation comprising:
a. exposing a population of cells to a gene editing method, wherein the gene editing method utilizes reagents configured to effect a mutation in a target sequence;
b. isolating single cells from the population;
c. providing a cell lysate from a single cell;
d. contacting the cell lysate with at least one amplification primer, at least one nucleic acid polymerase, and a mixture of nucleotides, wherein the mixture of nucleotides comprises at least one terminator nucleotide which terminates nucleic acid replication by the polymerase, and
e. amplifying the target nucleic acid molecule to generate a plurality of terminated amplification products, wherein the replication proceeds by strand displacement replication;
f. ligating the molecules obtained in step (e) to adaptors, thereby generating a library of amplification products;
g. sequencing the library of amplification products, and
h. comparing the sequences of amplification products to at least one reference sequence to identify at least one mutation.

2. The method of claim 1, wherein the at least one mutation is present in the target sequence.

3. The method of claim 1, wherein the at least one mutation is not present in the target sequence.

4. The method of claim 1, wherein the gene editing method comprising use of CRISPR, TALEN, ZFN, recombinase, meganucleases, or viral integration.

5. The method of claim 1, wherein the gene editing technique comprises use of a gene therapy method.

6. The method of claim 5, wherein gene therapy method is not configured to modify somatic or germline DNA of a cell.

7. The method of claim 1, wherein the reference sequence is a genome.

8. The method of claim 1, wherein the reference sequence is a specificity-determining sequence, where in the specificity-determining sequence is configured to bind to the target sequence.

9. The method of claim 8, wherein the at least one mutation is present in a region of a sequence differing from the specificity-determining sequence by at least 1 bases.

10. The method of claim 1, wherein the at least one mutation comprises an insertion, deletion, or substitution.

11. The method of claim 1, wherein the reference sequence is the sequence of a CRISPR RNA (crRNA).

12. The method of claim 1, wherein the reference sequence is the sequence of a single guide RNA (sgRNA).

13. The method of claim 1, wherein the at least one mutation is present in a region of a sequence which binds to catalytically active Cas9.

14. The method of claims 1, wherein at least some of the amplification products comprise a barcode.

15. The method of claims 1, wherein the method further comprises removing at least one terminator nucleotide from the terminated amplification products prior to ligation to adapters.

16. The method of claim 1, wherein the at least one mutation occurs in less than 1% of the population of cells.

17. The method of claim 1, wherein the at least one mutation occurs in no more than 0.0001% of the population of cells.

18. The method of claim 1, wherein the at least one mutation occurs in no more than 0.01% of the amplification product sequences.

19. The method of claim 1, wherein the at least one mutation is present in a region of a sequence not correlated with binding of a DNA repair enzyme.

20. The method of claim 1, wherein the at least one mutation is present in a region of a sequence not correlated with binding of MRE11.

21. The method of claim 1, wherein the method further comprises identifying a false positive mutation previously sequenced by an alternative off-target detection method.

22. The method of claim 21, wherein the off-target detection method is in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq.

23. The method of claim 1, wherein the single cell is a cancer cell.

24. The method of claim 1, wherein the single cell is a neuron or a glial cell.

25. The method of claim 1, wherein the single cell is a fetal cell.

26. A method of identifying specificity-determining sequences comprising:
a. providing a library of nucleic acids, wherein at least some of the nucleic acids comprise a specificity-determining sequence;
b. performing a gene editing method on at least one cell, wherein the gene editing method comprises contacting the cell with reagents comprising at least one specificity-determining sequence;
c. sequencing a genome of the at least one cell using the method of claim 1, wherein the specificity-determining sequence contacted with the at least one cell is identified; and
d. identifying at least one specificity-determining sequence which provides the fewest off-target mutations.

27. The method of claim 26, wherein the off-target mutations are synonymous or non-synonymous mutations.

28. The method of claim 26, wherein the off-target mutations are present outside of gene coding regions.

29. A method of in-vivo mutational analysis comprising:
a. performing a gene editing method on at least one cell in a living organism, wherein the gene editing method comprises contacting the cell with reagents comprising at least one specificity-determining sequence;
b. isolating at least one cell from the organism;
c. sequencing a genome of the at least one cell using the method of claim 1.

30. The method of claim 29, wherein the method comprises at least two cells.

31. The method of claim 30, further comprising identifying mutations by comparing the genome of a first cell with the genome of a second cell.

32. The method of claim 31, wherein the first cell and the second cell are from different tissues.

33. A method of predicting the age of a subject comprising:
a. providing at least one sample from the subject, wherein the at least one sample comprises a genome;
b. sequencing a genome using the method of claim 1 to identify mutations;
c. comparing mutations obtained in step b with a standard reference curve, wherein the standard reference curve correlates mutation count and location with a verified age; and
d. predicting the age of the subject based on the mutation comparison to the standard reference curve.

34. The method of claim 33, wherein the standard reference curve is specific for a subject's sex.

35. The method of claim 33, wherein the standard reference curve is specific for a subject's ethnicity.

36. The method of claim 33, wherein the standard reference curve is specific for a subject's geographic location where the subject spent a period of the subject's life.

37. The method of claim 33, wherein the subject is less than 15 years old.

38. The method of claim 33, wherein the at least one sample is more than 1000 years old.

39. The method of claim 33, wherein at least 5 samples are sequenced.

40. The method of claim 39, wherein the at least five samples are from different tissues.

41. A method for sequencing a microbial or viral genome comprising:
a. obtaining a sample comprising one or more genomes or genome fragments;
b. sequencing the sample using the method of claim 1 to obtain a plurality of sequencing reads; and
c. assembling and sorting the sequencing reads to generate the microbial or viral genome.

42. The method of claim 41, wherein the sample comprises genomes from at least ten organisms.

43. The method of claim 41, wherein the sample comprises genomes from at least 100 organisms.

44. The method of claim 41, wherein the sample origin is an environment comprising deep sea vents, ocean, mines, streams, lakes, meteorites, glaciers, or volcanoes.

45. The method of claim 41, further comprising identifying at least one gene in the microbial genome.

46. The method of claim 41, wherein the microbial genome corresponds to an unculturable organism.

47. The method of claim 46, wherein the microbial genome corresponds to an symbiotic organism.

48. The method of claim 41, further comprising cloning of the at least one gene in a recombinant host organism.
